(19) United States
(12) Patent Application Publication
(10) Pub. No.: US 2025/0262446 A1
(43) Pub. Date: Aug. 21, 2025

(54) BODY TISSUE RADIOFREQUENCY APPLICATORS, HEAD DEVICE AND USES THEREOF

(71) Applicant: NEUROCIA SAS, Montlaur (FR)

(72) Inventors: Sinisa JOVANOVIC, Novi Beograd (RS); Patricia LECCA, Montlaur (FR)

(21) Appl. No.: 19/055,896

(22) Filed: Feb. 18, 2025

Related U.S. Application Data
(63) Continuation-in-part of application No. PCT/EP2023/072875, filed on Aug. 18, 2023.
(63) Continuation-in-part of application No. PCT/EP2024/073241, filed on Aug. 19, 2024.

(30) Foreign Application Priority Data
Aug. 18, 2022 (EP) 22191009.4
Aug. 18, 2023 (EP) 23192285.7

(51) Int. Cl. A61N 1/40; A61N 5/06

(52) U.S. Cl. CPC A61N 1/40; A61N 5/0613; A61N 2005/0626; A61N 2005/0647; A61N 2005/0651; A61N 2005/0659; A61N 2005/0663

(57) ABSTRACT

The present invention relates to novel body tissue radiofrequency applicators (50), referred to as BTRFAs, as well as to a non-invasive medical head device useful for stabilizing and/or reversing the symptoms of neurodegenerative diseases and/or proteinopathies. The novel BTRFAs and medical head device are particularly useful for stabilizing and/or reversing the symptoms of Alzheimer's disease and/or Parkinson disease in a subject in need thereof.

### FIELD OF THE INVENTION

The present invention relates to novel body-tissue radiofrequency applicators and non-invasive medical head device useful for treating, preventing, stabilizing and/or reversing the symptoms of dementia and/or neurodegenerative diseases such as Alzheimer's disease, mild cognitive impairment, cerebral amyloid angiopathy Parkinson's disease, Lewy body dementia, vascular dementia, and/or frontotemporal dementia, in a subject in the need thereof. The present invention also relates to a novel body-tissue radiofrequency applicators and non-invasive head device useful for treating, preventing and/or alleviating traumatic brain injury (TBI), concussions, and other types of neurological conditions, in a subject in the need thereof. The medical head device according to the present invention is further useful for treating, preventing and/or mitigating depression, migraine headaches, myodesopsia, and/or tinnitus, in a subject in the need thereof. The present invention further relates to a wellness head device useful for general wellness purposes, particularly for relieving headaches and signs of fatigue, and/or enhancing general mental and cognitive abilities.

### BACKGROUND OF THE INVENTION

Several types of dementia are linked to neurodegenerative proteinopathies-diseases characterized by the accumulation of misfolded proteins in the brain. These changes disrupt the normal functioning of nerve cells and their connections, leading to their gradual degeneration. Alzheimer's disease and Parkinson's disease are among the most common neurodegenerative proteinopathies. While these conditions share some underlying pathological mechanisms, the specific psychological and physiological symptoms vary depending on the affected brain regions.

Currently, there are no cures for these neurodegenerative diseases. Most available drugs have proven ineffective, as they neither improve nor prevent the symptoms associated with these disorders. Additionally, many treatments fail to cross the blood-brain barrier or have been found to be toxic. As a result, current medical interventions focus primarily on alleviating symptoms rather than addressing the underlying disease.

Several non-drug approaches have been explored, including non-invasive brain stimulation (NIBS) and deep brain stimulation (DBS). NIBS encompasses a range of technologies and techniques that use various patterns of electrical, magnetic, electromagnetic, sound, red, and/or near-infrared (NIR) stimulation transcranially—meaning noninvasively, through the scalp—to modify brain activity and influence large-scale neural networks. These techniques do not require breaking the skin and are designed to alter brain function from the surface. NIBS includes methods such as electroconvulsive therapy (ECT), transcranial magnetic stimulation (TMS), repetitive transcranial magnetic stimulation (rTMS), transcranial alternating current stimulation (tACS), cranial electrotherapy stimulation (CES), transcranial direct current stimulation (tDCS), transcranial electromagnetic treatment (TEMT), repeated electromagnetic field shock (REMFS), and photobiomodulation (PBM).

One of the key challenges with NIBS-based medical devices is that their components—such as coils, antennas, lasers, or LEDs, which generate electrical, magnetic, electromagnetic, or red/NIR stimulation signals—are often not designed for efficient transcranial administration in humans. These components tend to be too large and bulky for the average human head, failing to provide precise cortical targeting or sufficient signal penetration within the cortex. The size and shape of the antennas and coils in current NIBS head devices are not conducive to regular, convenient use, particularly in home settings. As a result, many existing NIBS devices are large, cumbersome apparatuses that require patients to visit hospitals regularly for transcranial brain stimulation treatments. This is not only inconvenient but often impractical for patients suffering from neurodegenerative diseases, who may find it difficult or even impossible to access such treatments on a regular basis.

Another challenge is that the components of NIBS-based medical devices must efficiently deliver energy into human tissue—such as the head—in a manner that ensures homogeneous distribution and sufficient depth of penetration into cerebral structures. Additionally, these components must be adaptable to the curvature of body tissues, whether it is the human head or other parts of the body, without compromising power efficiency, minimizing energy emission into free space. This is particularly important when the brain of a human subject is targeted.

The present invention represents a significant advancement in the field, offering components such as body tissue applicators and non-invasive medical head devices that effectively and reliably deliver electromagnetic signals or a combination of neurostimulatory signals with high efficiency in terms of absorption and penetration within human tissue, particularly the head. These devices ensure a reliable, homogeneous, and efficient specific absorption rate (SAR), crucial for therapeutic efficacy. The non-invasive medical or wellness head device of the present invention is available in the form of a head device, headset, or compact headcap. Not only is it lightweight and suitable for regular home use, but it also generates a consistent and homogeneous transcranial signal, ensuring effective coverage and penetration within the patient's cortex. Consequently, this device can deliver a therapeutically effective dosage to brain cells, regardless of the individual's anatomical variations. The non-invasive medical head device is designed to stabilize and/or reverse the symptoms of neurodegenerative diseases and proteinopathies, particularly in conditions such as Alzheimer's disease and Parkinson's disease.

### SUMMARY OF THE INVENTION

The present invention thus provides novel body tissue radiofrequency applicators (50) and non-invasive medical device comprising said one or more of said body tissue radiofrequency applicators which are specifically designed for directly administering a homogeneous pulsed electromagnetic signal to a specific body tissue, such as the head or the abdomen, of a human subject.

The present invention also provides a novel non-invasive medical or wellness device comprising a synergistic combination of the body tissue radio frequency applicators with LEDs for simultaneous emissions of signals of pulsed electromagnetic waves in combination with red and near-infrared lights or signals.

The non-invasive medical device according to the present invention is preferably in the form of a head-mounted wearable non-invasive device which may be placed on the head, in direct contact with the scalp and/or skull of a subject, thereby allowing homogeneous and reliable exposure of the cortex of the subject to said electromagnetic waves in combination with red and near-infrared signals or lights.

Novel body tissue radiofrequency applicators and non-invasive medical head device according to the present invention are particularly useful for treating and/or preventing neurodegenerative diseases as well as for stabilizing and/or reversing the symptoms of neurodegenerative diseases such as Alzheimer's disease, mild cognitive Impairment (MCI), Parkinson's disease, cerebral amyloid angiopathy, dementia with Lewy bodies (DLB), and frontotemporal dementia in a subject in the need thereof. The present invention also relates to novel body tissue radiofrequency applicators and/or non-invasive medical head device for treating and/or preventing and/or alleviating symptoms of traumatic brain injury (TBI), concussions (mild TBI) and other types of neurological conditions in a subject in the need thereof. The present invention further relates to novel body tissue radiofrequency applicators and/or non-invasive medical head device for preventing and/or reducing or eliminating depression or the symptoms of depression, delaying, reducing and/or eliminating depression and its symptoms, reducing and/or relieving migraines, reducing and/or mitigating myodesopsia and vitreous opacities, and/or tinnitus (hardness of hearing) in a subject in the need thereof. Finally, the novel body tissue radiofrequency applicators and/or non-invasive head device according to the present invention may be used by healthy patients for general wellness purposes, particularly for relieving headaches and signs of fatigue, and/or enhancing general mental and cognitive abilities.

### BRIEF DESCRIPTION OF THE FIGURES

FIGS. 1A-B: (A) shows a complete schematic view of the X-BTRFA with a coaxial connector structure according to FIG. 2 and with meandered antenna wire. (B) shows a further schematic view of the body tissue radio frequency applicator or antenna (BTRFA) according to the present invention with an X-type of crisscrossed connection between the coaxial conductors and the central antenna wires. The antenna wires are shown only schematic and simplified as two loops.

FIGS. 2A-B: (A) shows a detailed view of the X-BTRFA with ends of the antenna wires and the central coaxial connector structure. (B) shows a detailed view (top view on the left and bottom view on the right) of the X-BTRFA in an alternative embodiment to FIG. 2A in a connection to a PCB.

FIGS. 3A-B: (A) is a graph of the S₁₁-parameter (scattering parameter or reflection: return loss—RL) of X-BTRFA surrounded by free space or air and operating at 915 MHz. (B) is a graph showing the power distribution of X-BTRFA surrounded by free space.

FIG. 4: shows antenna gain of X-BTRFA surrounded by free space at 0.915 GHz.

FIG. 5: is a schematic of the X-BTRFA placed close to body tissue. Dimensions of the body tissue 132×105×40 mm are indicated by way of examples and 3 mm is the distance between the surface of the body tissue and the X-BTRFA.

FIGS. 6A-B: (A) is a graph of the S₁₁-parameter (scattering parameter or reflection: return loss—RL) of X-BTRFA placed close to body tissue. (B) is a graph showing the power distribution of X-BTRFA placed close to body tissue.

FIG. 7: is a simulation of the SAR(1 g) for X-BTRFA placed close to body tissue at 0.915 GHz.

FIGS. 8A-B: (A) shows a complete view of the Y-BTRFA of FIG. 8B with a coaxial connector structure according to FIG. 9 and with meandered antenna wire. (B) shows a further schematic view of the body tissue radio frequency applicator or antenna (BTRFA) according to the present invention with a Y-type of crisscrossed connection between the coaxial conductors and the central antenna wires. The antenna wires are shown only schematic and simplified as two non-meandered loops.

FIGS. 9A-B: (A) shows a detailed view of the Y-BTRFA with ends of the antenna wires and the central coaxial connector structure. (B) shows a detailed view (top view on the left and bottom view on the right) of the Y-BTRFA in an alternative embodiment to FIG. 9A in a connection to PCB.

FIGS. 10A-B: (A) is a graph of the S₁₁-parameter (scattering parameter or reflection: return loss—RL) of Y-BTRFA surrounded by free space or air and operating at 915 MHz. (B) is a graph showing the power distribution of Y-BTRFA surrounded with free space (air).

FIG. 11: is a simulation showing the antenna gain of Y-BTRFA surrounded by free space at 0.915 GHz.

FIG. 12: is a schematic of the Y-BTRFA placed close to body tissue. Examples of dimensions of the body tissue 132×105×40 mm were provided and 3 mm may be an exemplary distance between the surface of the body tissue and the Y-BTRFA.

FIGS. 13A-B: (A) is a graph of the S₁₁-parameter (scattering parameter or reflection: return loss—RL) of Y-BTRFA placed close to body tissue. (B) is a graph showing the power distribution of Y-BTRFA placed close to body tissue.

FIG. 14: is a simulation of the SAR(1 g) for Y-BTRFA placed close to body tissue at 0.915 GHz.

FIGS. 15A-C: (A) is a schematic of BTRFA (either X-BTRFA or Y-BTRFA) printed on a thin flexible PCB (“printed circuit board”). The size of the body tissue sample is 132 mm×105 mm×40 mm, while the PCB size is 92 mm×81 mm with thickness of 0.1 mm. BTRFA is realized with printed conductive line with thickness of 0.0175 mm (17.5 μm) and width of 1 mm. Thin flexible PCB with printed BTRFA is placed at the 3 mm distance above the body tissue sample. (B) and (C) are schematics of the connections between the coaxial cable and printed PCB metal trace for the X connection on the left (B), and for the Y connection on the right (C).

FIGS. 16A-B: (A) represents the simulation S11 parameter results of printed X-BTRFA close to the body tissue. (B) represents simulation S11 parameter results of printed Y-BTRFA close to the body tissue.

FIGS. 17A-B: (A) represents the power distribution results of printed model of X-BTRFA close to body tissue. (B) represents the power distribution results of printed model of Y-BTRFA close to body tissue.

FIGS. 18A-B: (A) shows a Specific Absorption Rate (SAR) simulation for printed X-BTRFA above body tissue. The simulation shows body tissue volume with SAR value of at least 2 W/kg. The SAR value of 2 W/kg or greater covers a wider area within the body tissue, but with a lesser penetration depth at the center of the X-BTRFA, when compared to the Y-BTRFA SAR simulation in 18B. The maximum SAR value is 9.51 W/kg at four places indicated by black dots. (B) shows a Specific Absorption Rate (SAR) simulation for printed Y-BTRFA above body tissue. The simulation shows body tissue volume with SAR value of at least 2 W/kg or greater also covers a wide area within the body tissue, but narrower than the area covered when using X-BTRFA and with a higher penetration depth within the body tissue at the center of the Y-BTRFA, when compared to penetration depth when using X-BTRFA in 18A. The maximum SAR value of 11.9 W/kg is at the center of Y-BTRFA.

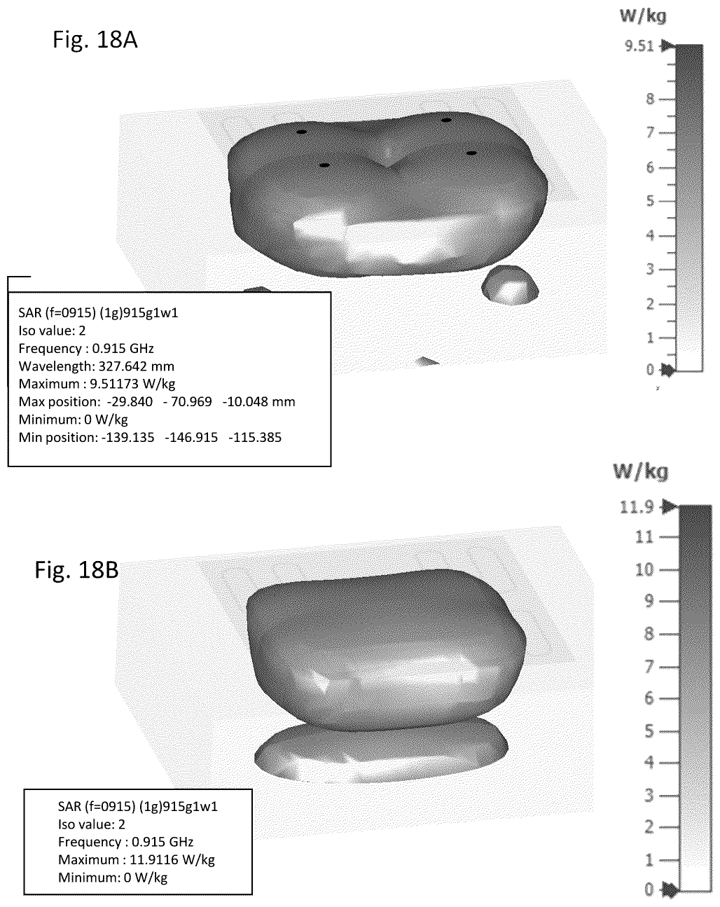

FIGS. 19A-C: are schematics of the slight possible variations of the BTRFA extremities which may be further rounded up to accommodate the LEDs as shown in FIG. 20. Another advantage of using differently meandered BTRFAs is to optimize the SAR coverage of a human head function of shapes and sizes thereof, especially considering that the overall device is intended to be suitable for various human head sizes.

FIG. 20: is a schematic of one BTRFA unit carrying 6 LEDs by way of example. The combination of the BRTFA unit and LEDs is viewed from below; the LEDs being directed towards and placed at proximity to the head. The meandered shape of the BTRFA unit is thus convenient to accommodate several LEDs or any other elements of the device. Particularly, it is useful if the shape of BTRFAs is accommodated in accordance with the LEDs positions to avoid the areas with the maximum red and NIR light intensity. For the best red/NIR light coverage, LEDs may be arranged around a head at as equal distance as possible. Examples of distances in between the various LEDs are provided in the figures in mm.

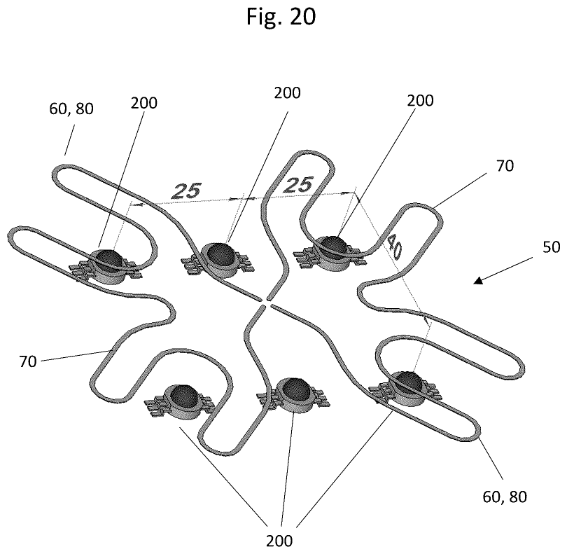

FIGS. 21A-B: (A) shows a complete view of the Z-BTRFA of FIG. 21B with a coaxial connector structure according to FIG. 22 and with simple looped antenna wire. (B) shows a further schematic view of the body tissue radio frequency applicator or antenna (BTRFA) according to the present invention with a Z-type of connection between the coaxial conductors and the central antenna wires. The antenna wires are shown only schematic and simplified as two loops, each loop having the length of about λ₀/2@f_{c} (half of the wavelength at the intended working frequency) unlike X-BTRFA and Y-BTRFA that have two times larger loop lengths of λ₀@f_{c}.

FIGS. 22A-B: (A) shows a detailed view of the Z-BTRFA with ends of the antenna wires and the central coaxial connector structure. (B) shows a detailed view (top view on the left side and bottom view on the right side) of the Z-BTRFA in a connection to PCB as an alternative embodiment to FIG. 22A.

FIGS. 23A-B: (A) is a graph of the S₁₁-parameter (scattering parameter or reflection: return loss—RL) of Z-BTRFA surrounded by free space or air and operating at 915 MHz. (B) is a graph showing the power distribution of Z-BTRFA surrounded with free space or air.

FIG. 24: is a simulation showing the antenna gain of Z-BTRFA surrounded by free space or air at 0.915 GHz.

FIG. 25: is a schematic of the Z-BTRFA placed close to body tissue, for example positioned at 3 mm distance above the body tissue sample. Dimensions of the body tissue 132×105×40 mm are provided by way of example.

FIGS. 26A-B: (A) is a graph of the S₁₁-parameter (scattering parameter or reflection: return loss—RL) of Z-BTRFA placed close to body tissue. (B) is a graph showing the power distribution of Z-BTRFA placed close to body tissue.

FIG. 27: is a simulation of the SAR(1 g) for Z-BTRFA placed close to body tissue at 0.915 GHz.

FIG. 28: a schematic of a curved or bent BTRFA (either X-BTRFA or Y-BTRFA) above curved multi-layer tissue sample, such as a human head, showing from top to bottom with respective thickness: skin (5 mm); skull (6 mm); CSF (cerebrospinal fluid) (3 mm), and the brain grey matter (40 to 47 mm).

FIGS. 29A-B: (A) is a graph of the S₁₁-parameter (scattering parameter or reflection: return loss—RL) of curved or bent X-BTRFA above a curved multi-layer tissue sample, such a human head. (B) is a graph of the S11-parameter (scattering parameter or reflection: return loss—RL) of curved or bent Y-BTRFA above a curved multi-layer tissue sample, such a human head.

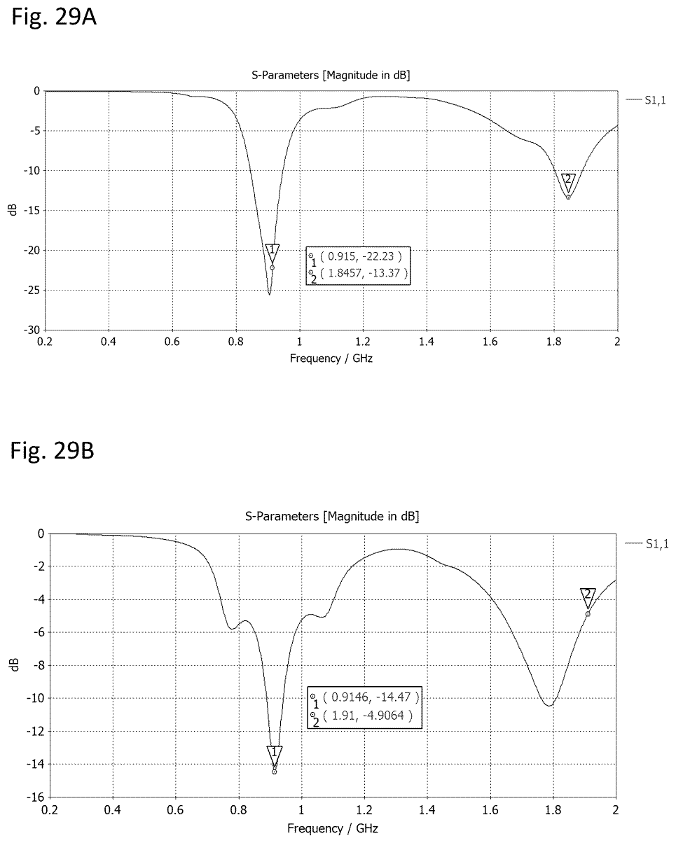

FIGS. 30A-B: (A) is a graph of the power distribution of curved X-BTRFA placed close to body tissue. (B) is a graph of the power distribution of curved X-BTRFA placed close to multi-layer body tissue.

FIGS. 31A-B: (A) is a graph of the power distribution of curved Y-BTRFA placed close to body tissue. (B) is a graph of the power distribution of curved Y-BTRFA placed close to multi-layer body tissue.

FIGS. 32A-B: (A) is a simulation of the SAR(1 g) for curved or bent X-BTRFA placed close to body tissue at 0.915 GHz. (B) is a simulation of the SAR(1 g) for curved or bent Y-BTRFA placed close to body tissue at 0.915 GHz.

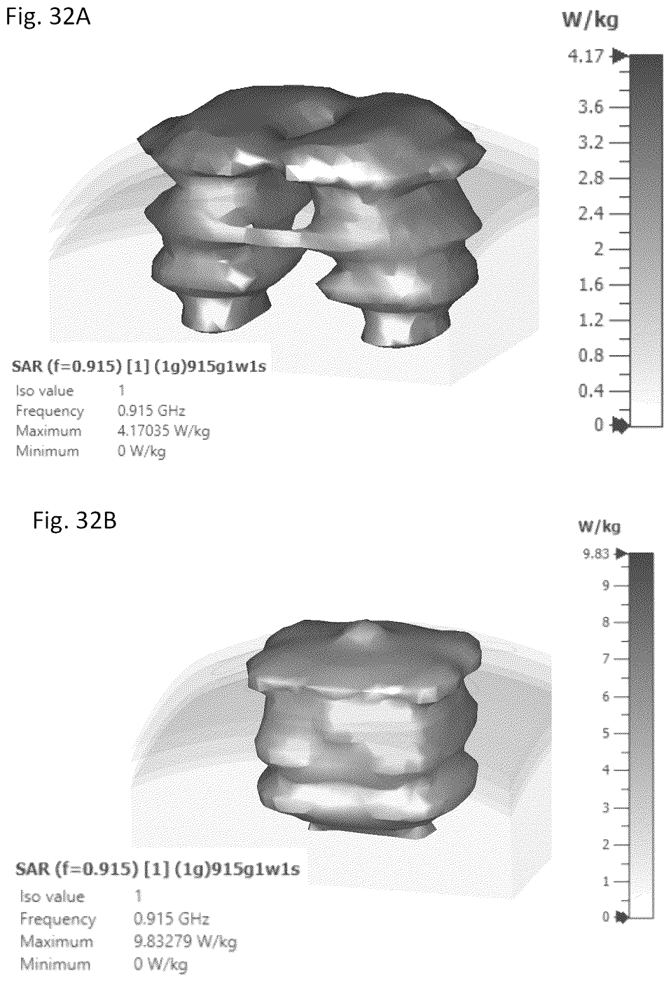

FIGS. 33A-B: are schematics of an exemplary arrangement of eight BTRFAs, two of each BTRFA unit being positioned on each lobe of the brain, namely frontal, parietal, occipital lobes and one BTRFA on each side covering each temporal lobe. (A) is a top view of the head. (B) is a top view of the head.

FIGS. 34A-B: are simulations of the SAR (1 g) obtained when using the arrangement of array of eight BTRFAs as shown in FIG. 33. (A) is a semi-front view of the SAR simulation. (B) is a side view of the SAR simulation.

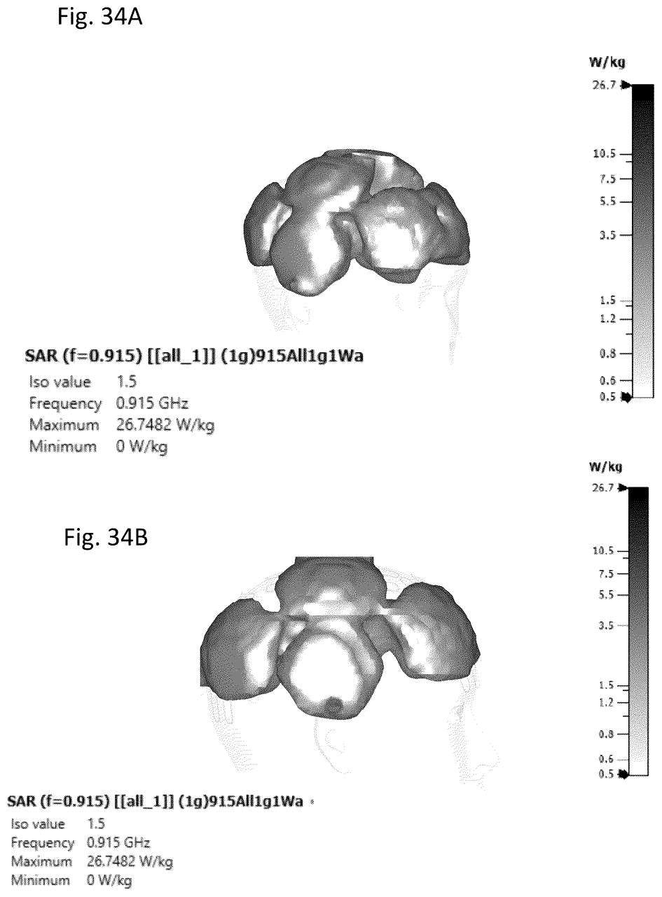

FIGS. 35A-B: show (A) a schematic of a 3-in-1 LED, and (B) the graph of the Relative Luminous Intensity vs angle of such 3-in-1 LED.

FIG. 36: is a schematic showing an example of the synchronization of the electromagnetic signals 915 MHz with a repetition rate of 200 Hz and a duty cycle of 100% as delivered by BTRFA with RED/NIR signals (at the three wavelengths 660 nm, 810 nm and 1064 nm) as delivered by LEDs with a repetition rate of 40 Hz and a duty cycle of 12.5%. The frequency of the electromagnetic signals at 915 MHz has sinusoidal signal with 574875 periods within every 0.625 ms wide pulse.

FIG. 37: is a schematic showing an example of the synchronization of the electromagnetic signals 915 MHz with a repetition rate of 200 Hz and a duty cycle of 100% as delivered by BTRFA with RED/NIR signals (at the three wavelengths 660 nm, 810 nm and 1064 nm) as delivered by LEDs with a repetition rate of 40 Hz and a duty cycle of 12.5%. The frequency of the electromagnetic signals at 915 MHz has sinusoidal signal with 574875 periods within every 0.625 ms wide pulse.

FIG. 38: is a schematic showing an example of the synchronization of the electromagnetic signals 915 MHz with a repetition rate of 200 Hz and a duty cycle of 100% as delivered by BTRFA with RED/NIR signals (at the three wavelengths 660 nm, 810 nm and 1064 nm) as delivered by LEDs with a repetition rate of 40 Hz and a duty cycle of 12.5%. The frequency of the electromagnetic signals at 915 MHz has sinusoidal signal with 574875 periods within every 0.625 ms wide pulse.

FIGS. 39A-C: is a similar schematic of the example of the combination of one BTRFA (50) unit carrying six LEDs (200) as shown in FIG. 20, but also showing (5) the output amplifier; (11) the PCB for output amplifier and BTRFA; (12) the PCB for LED; (14) the Power supply and control lines; (15) the LED power lines; and (16) the RF signal coaxial cable. (A) is a view from below (the LEDs shining towards the body tissue), and (B) is a side view of the same combination. (C) is a closer look the (11) PCB output amplifier and BTRFA showing in more details (5) the output amplifier; (14) the Power supply and control lines; and (16) the RF signal coaxial cable

FIGS. 40A-B: are schematics of the medical head device as shown in FIG. 33, but also showing the combinations of the array of BTRFA with the array of LEDs, each BTRFA carrying for example six LEDs, as well as (9) the central unit PCB; (10) the Power supply PCB; (11) the PCB for output amplifier and BTRFA; and (12) the PCB for LED. (A) is a side view of the medical device positioned on the head and (B) is a top view of the medical device positioned on the head.

FIGS. 41A-B: are schematics of an exemplary arrangement of the arrays of combined units of BTRFAs and LEDs as shown in FIG. 20. The simulation of RED/NIR light exposure on the head as shown (A) in a front view and (B) in a back view, clearly shows a homogeneous coverage of the RED/NIR light on the whole head.

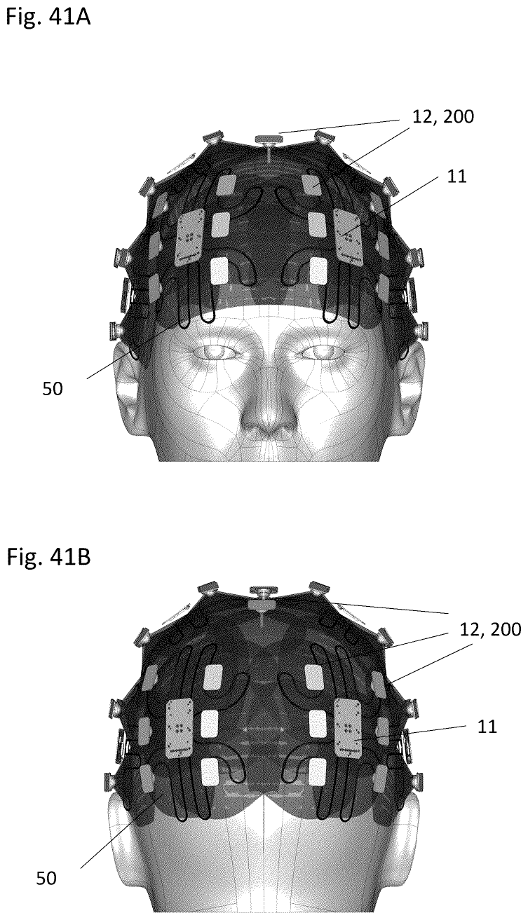

FIGS. 42A-C: correspond to different views of the schematics and RED/NIR simulations shown in FIG. 41. (A) is a side view of the head; (B) is a top view of the head; (C) is a semi-side view of the head.

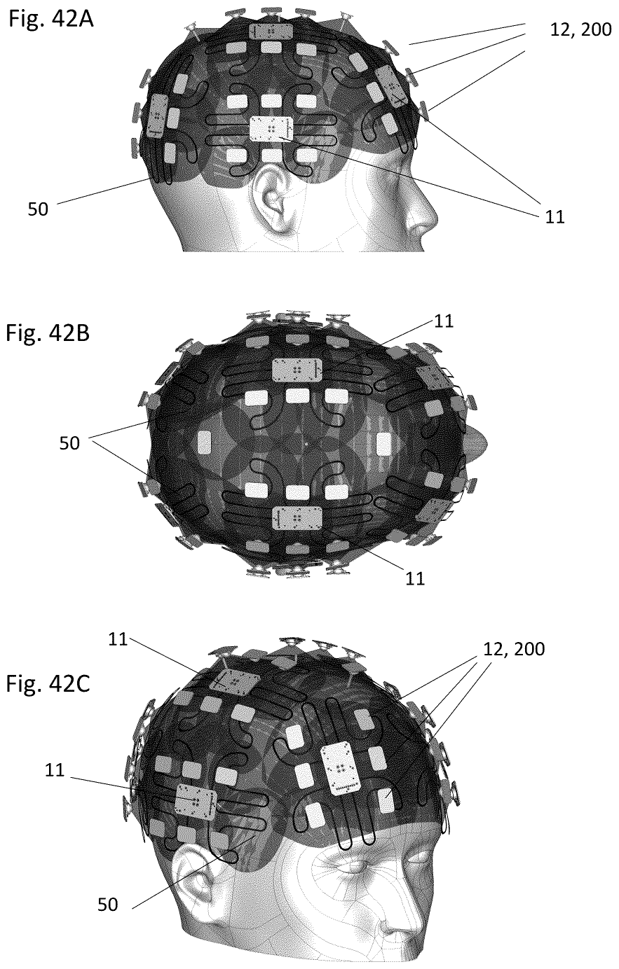

FIG. 43: is a block diagram of the entire electronic circuit of the medical head device according to the present invention with eight BTRFA, each of the BTRFA carrying six LEDs. (1) corresponds to RF generator (VCO—Voltage Controlled Oscillator); (2) corresponds to amplifiers; (3) corresponds to the Resistive RF power divider (1 to 2); (4) corresponds to the Resistive RF power divider (1 to 4); (5) corresponds to the Output amplifiers; 60 corresponds to BTRFA; (200) corresponds to LEDs (either single LEDs or 3-in-1 CHIP LEDs); (8) corresponds to Controller; (9) corresponds to the Central unit PCB; (10) corresponds to the Power supply PCB; (11) corresponds to PCB for output amplifier and BTRFA; (12) corresponds to the PCB for LED; and (13) corresponds to the External Battery.

FIGS. 44A-B: are schematic views of large highly meandered X-BTRFA (50) working at 50-100 MHz made of wire or flexy PCB (60), having two halves (70) and X-type central connection (90). (A) shows the X-BTRFA with X-type central connection realized on a separate piece of rigid PCB (which is a part of a larger PCB containing output RF amplifier), while (B) shows the X-BTRFA placed close to a rectangular sample of body tissue, for example positioned at 3 mm distance above the body tissue sample.

FIGS. 45A-C: shows a schematic views of the structure of a highly meandered BTRFA operating in the 50-100 MHz frequency range. This BTRFA is made on a flexible PCB with a thin dielectric layer (60) containing a long elastic central strip (61) with 92 elastic branches (62) orthogonally attached to the central strip. The dielectric layer serves as a base for two identical halves of conductive meandered transmission lines (70) that form the BTRFA structure. The conductive lines (72) that form the BTRFA consist of a thin layer of copper with a width of 0.7 mm and a thickness of 0.035 mm. The central elastic strip (61) allows the BTRFA structure to bend along its longer axis, as shown in FIG. 45B, while the elastic orthogonal branches enable bending in the plane orthogonal to the longer axis of the BTRFA as shown in FIG. 45C. Combining these two types of bending allows the BTRFA to conform to the curvature of the human head and to be positioned at an approximately constant distance (around 3 to 5 mm) from the curved surface of the head.

FIGS. 46A-B: (A) shows a bottom detailed view of the central coaxial connector structure of a large PCB BTRFA (50) working at 50-100 MHz. The central PCB connection shown in (A) is X-type and in (B) is Y-type. Both PCB connections comprise a printed circuit board comprising an RF input microstrip line (180), a first electrically conductive layer (190), a second electrically conductive layer (210) and a dielectric layer (220) separating the first and second electrically conductive layer (190, 210). End points A1, A2, B1 and B2 of the two PCBs or wires 80 in each of the two halves (70) are connected to either an RF input microstrip line 180 or to a top GND layer 190 at the connection points (160). (B) shows a zoomed out bottom view of the large meandered BTRFA made of PCB with the central coaxial connector switch.

FIGS. 47A-C: (A) shows one quarter of the BTRFA operating in the 50-100 MHz frequency range. The figure illustrates how the conductive lines (72) meander along the edges of the dielectric elastic branches (62). The dielectric substrate (60) has mechanical vias (64) that serve to attach the BTRFA to the housing in which it is placed. This housing can be made of either fabric or lightweight plastic. (B) shows the central part of the BTRFA with four connection points (A1, A2, B1, and B2) to which the RF excitation signal is applied. There are metallized vias (74) on the conductive lines (72). These vias allow the total length of the BTRFA transmission line to be shortened and its operating frequency to be changed by appropriately connecting the corresponding pairs of vias (which are symmetrical with respect to the center of the BTRFA) using pairs of conductive shorting connectors (76), as shown in (C).

FIG. 48: shows the frequency characteristic of the reflection coefficient obtained from the electromagnetic simulation of the BTRFA structure shown in FIG. 44B, indicating good matching at 48.94 MHz and 95 MHz.

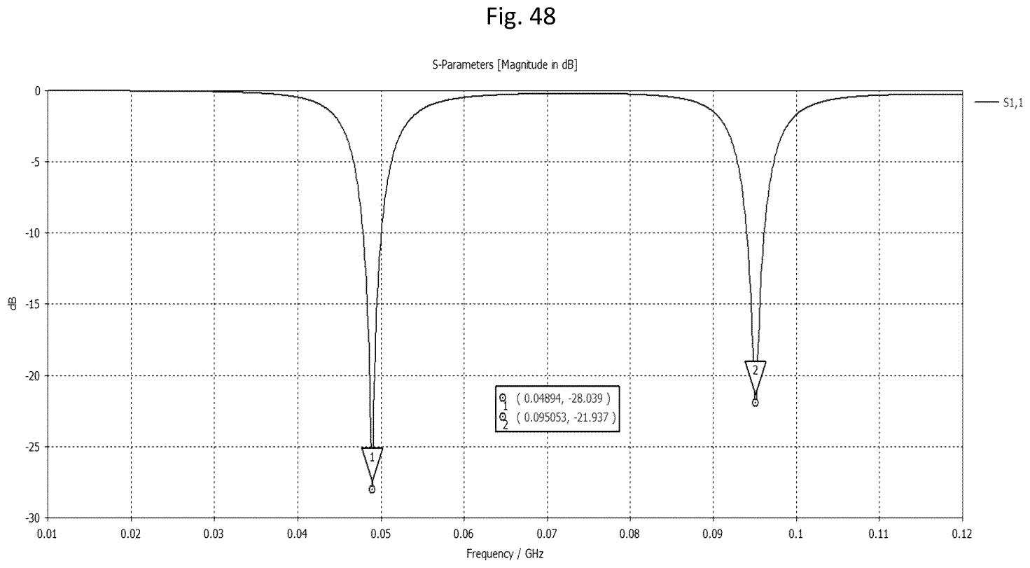

FIGS. 49A and 49B: show the power distribution of the same BTRFA at the resonant frequencies of 48.94 MHz and 95 MHz. FIG. 49A indicates that at 48.94 MHz (marked with a vertical line in FIG. 49A), the BTRFA placed close to body tissue accepts 88.8% (0.444/0.5) of the total available power (0.5 W), while about 11.11% (0.0555/0.5) is reflected back to the RF generator. Approximately 69.99% (0.349/0.5) of the power accepted by the BTRFA is delivered to the body tissue. The metal loss is about 18.92% (0.0496/0.5), which is higher than for the BTRFA at 915 MHz due to the very long and thin conductive line. FIG. 49B shows that at 95 MHz (marked with a vertical line in FIG. 49B), the BTRFA placed close to body tissue accepts 80.2% (0.401/0.5) of the total available power (0.5 W), while about 19.8% (0.09899/0.5) is reflected back to the RF generator. Approximately 58.5% (0.2925/0.5) of the power accepted by the BTRFA is delivered to the body tissue

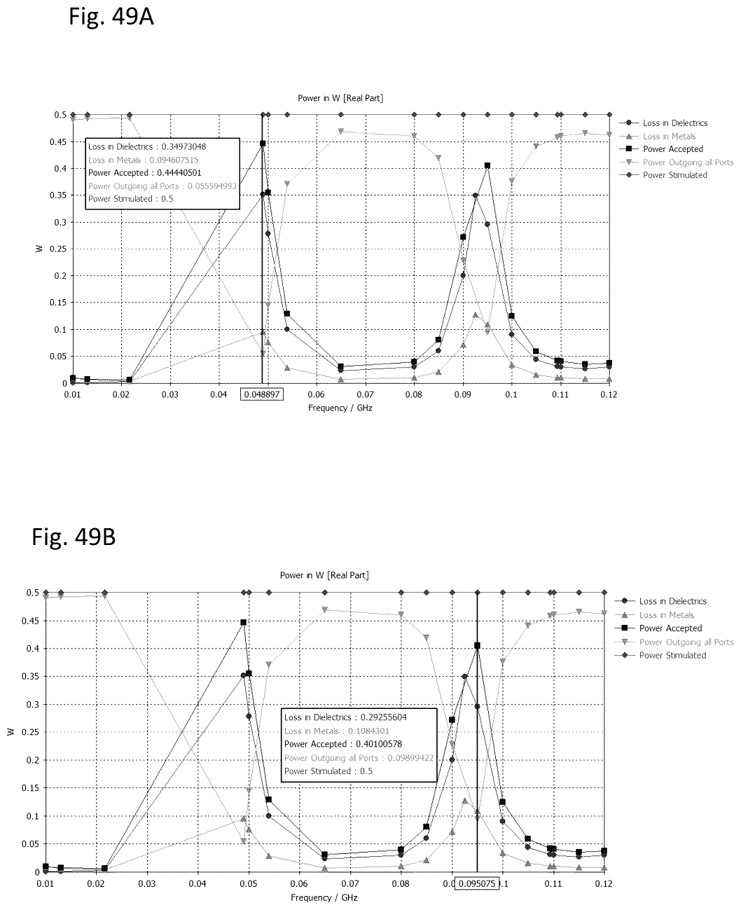

FIG. 50: shows the power distribution for the BTRFA surrounded by free space. It can be seen that at both frequencies (around 50 MHz and around 100 MHz, marked by vertical lines) where resonance occurred when the BTRFA was near a sample of body tissue the entire RF signal power was reflected back to the generator, as was the case with the BTRFA designed for 915 MHz. This demonstrates that all presented BTRFAs achieve efficient RF energy transfer only to the body tissue in their immediate vicinity.

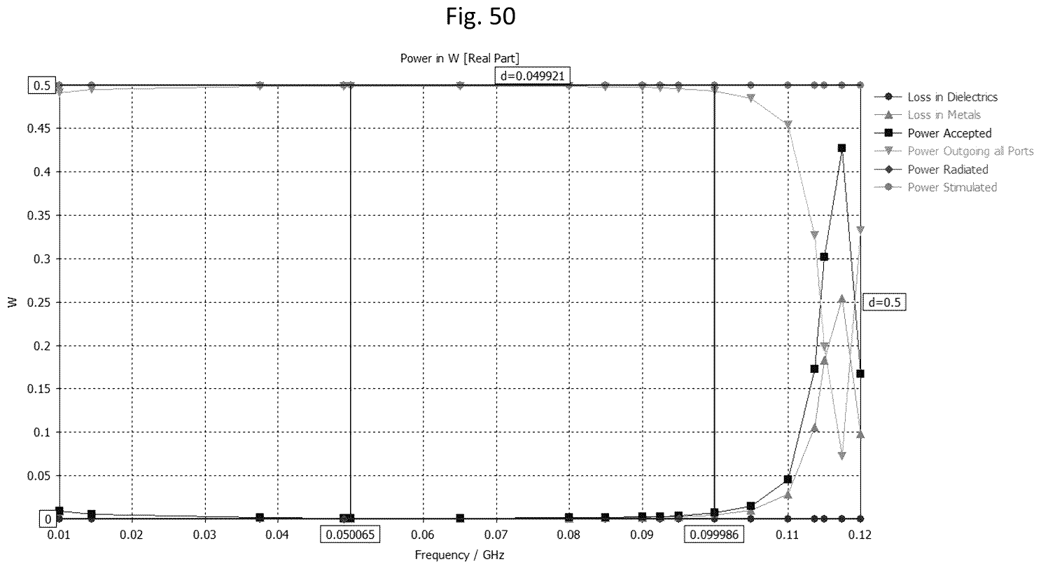

FIGS. 51A-B: are schematics of an exemplary arrangement of two large meandered BTRFAs working at 50-100 MHz, each BTRFA unit being positioned on each side of the head, thereby covering the brain, namely frontal, parietal, occipital lobes. (A) is a side view of the head. (B) is a front view of the head. 52A is a top view of the head, 51B is a right view of the head.

FIGS. 52A-B: are schematics of an exemplary arrangement of two large meandered BTRFAs working at 50-100 MHz, each BTRFA unit being positioned on each side of the head, thereby covering the brain, namely frontal, parietal, occipital lobes. (A) is a top view of the head. (B) is a side view of the head.

FIG. 53: shows the SAR distribution of the signal at 50 MHz achieved using two BTRFAs arranged as shown in FIGS. 51 and 52, around a simplified model of the human head. The generator power is set so that the maximum SAR value is limited to 2 W/kg (1 g). (A) shows the shaded area where the SAR value exceeds 0.5 W/kg (1 g), while (B) shows the shaded area where the SAR value exceeds 0.2 W/kg (1 g). It can be seen that the presented BTRFAs achieve a very deep penetration of the RF signal at 50 MHz.

FIG. 54: shows the SAR distribution of the signal at 100 MHz on the same model. The generator power is set so that the maximum SAR value is limited to 2 W/kg (1 g). (A) shows the shaded area where the SAR value exceeds 0.2 W/kg (1 g) using the X type BTRFA. (B) shows the shaded area where the SAR value exceeds 0.2 W/kg (1 g) using the Y type BTRFA. A solid penetration depth is achieved, but it is somewhat lower than in the case of stimulation with the same BTRFA at 50 MHz.

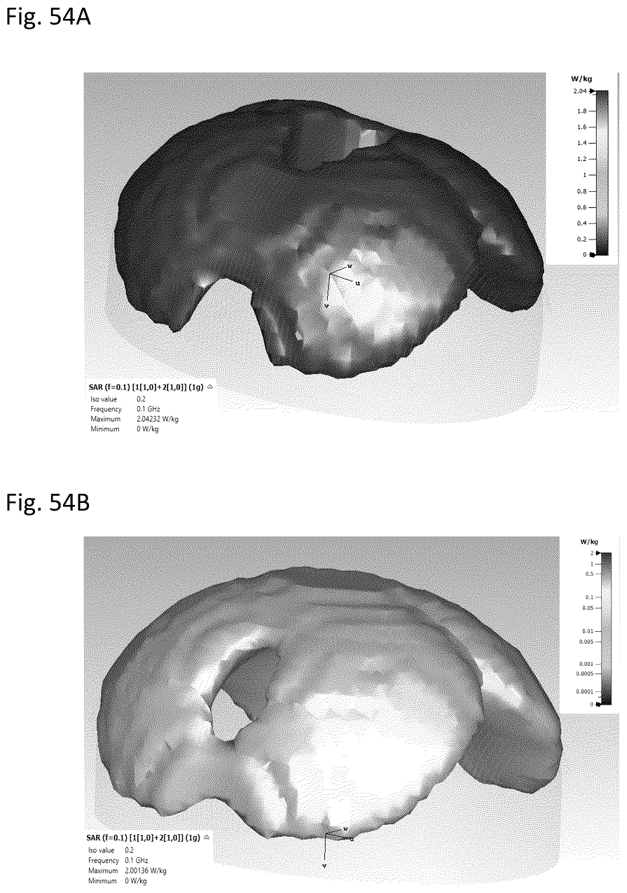

FIGS. 55A-B: are schematics of the external global design the non-invasive head device of the present invention which is present in the form of a compact hat (or “augmented hat”). (A) is a side view and (B) is a top view.

### DETAILED DESCRIPTION

According to a first aspect, the present invention provides body tissue applicators (50) which are radiofrequency (RF) applicators specifically designed to apply an electromagnetic field or waves inwardly directly to human body tissue. These body tissue radiofrequency applicators (50) are referred hereinafter as BTRFA in the singular or as BTRFAs in the plural.

Body tissue radiofrequency applicators (BTRFAs) (50) according to the present invention are configured for emitting a pulsed electromagnetic signal within the radiofrequency (RF) spectrum—particularly within the very high frequency spectrum (VHF) or ultra-high frequency (UHF) spectrum to adjacent human tissue. These BTRFAs operate at a single working frequency or within a narrow frequency band ranging from 30 MHz to 3000 MHz, with a repetition or a pulsation rate between 10 Hz and 400 Hz. In particular the BTRFAs (50) operate within 30-300 MHz (VHF) or within 300-3000 MHz (UHF). The BTRFAs (50) may be configured to operate at either a fixed single frequency or within a narrowband working frequency within this spectrum. Alternatively, they may be configured to operate at multiple working frequencies within the RF spectrum, thereby delivering pulsed RF electromagnetic signals across multiple frequencies between 30 MHz and 3000 MHz to the targeted human tissue.

Preferred RF frequencies emitted by the BTRFAs according to the present invention may range from 30-300 MHz, 30-200 MHz, 40-250 MHz, 50-200 MHz, 50-150 MHz, 100-150 MHz, 60-100 MHz, 50-70 MHz, 60-65 MHz, 400-1500 MHz, 500-1300 MHz, 800-1100 MHz, 850-950 MHz, or 800-930 MHz, or 850-915 MHz, 910-920 MHz.

The electromagnetic energy or RF signal is also pulsed with a repetition or a pulsation rate within the range of 10-300 Hz, 20-200 Hz, 40-100 Hz, preferably at around 20 Hz, 40 Hz, 100 Hz, or at around 200 Hz.

Most preferred electromagnetic energy signals have a frequency around 50-100 MHz, around 60 MHz, around 900-915 MHz, or around 910-920 MHz and is pulsed with a cycle of repetition around 40-100 Hz, 100-200 Hz, 40 Hz, 100 Hz, or 200 Hz.

The body tissue radiofrequency applicators (50) may be made or constructed from wire or printed circuit board (PCB) structures, with PCBs being the preferred material. The wire or printed circuit board (PCB) structures of the BTRFAs (50) may be flat or flexible curved, with a preference for curved wire and flexible PCB structures (60) to facilitate positioning adjacent to or in proximity to human tissue. The BTRFA may be divided into an arrangement of at least two sections or two halves (70), wherein the wire or PCB (60) in each section or half (70) is connected to a central connector structure (90), and wherein the wire or PCB (60) of each section or halve (70) comprises a first and a second end (80).

As indicated above, the BTRFA may be divided into an arrangement of at least two sections or two halves (70), and the present invention provides several embodiments of said arrangement of the at least two sections or halves (70).

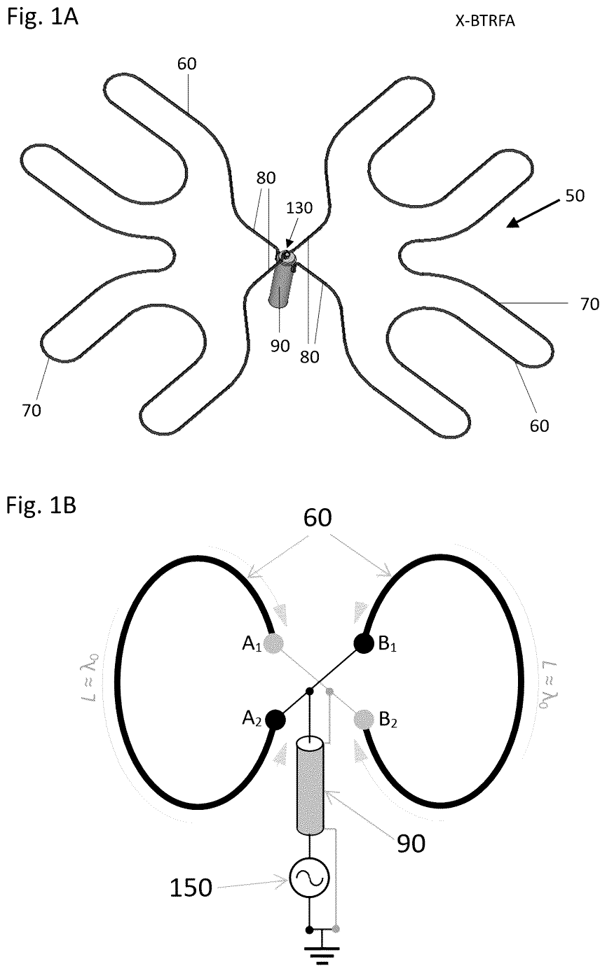

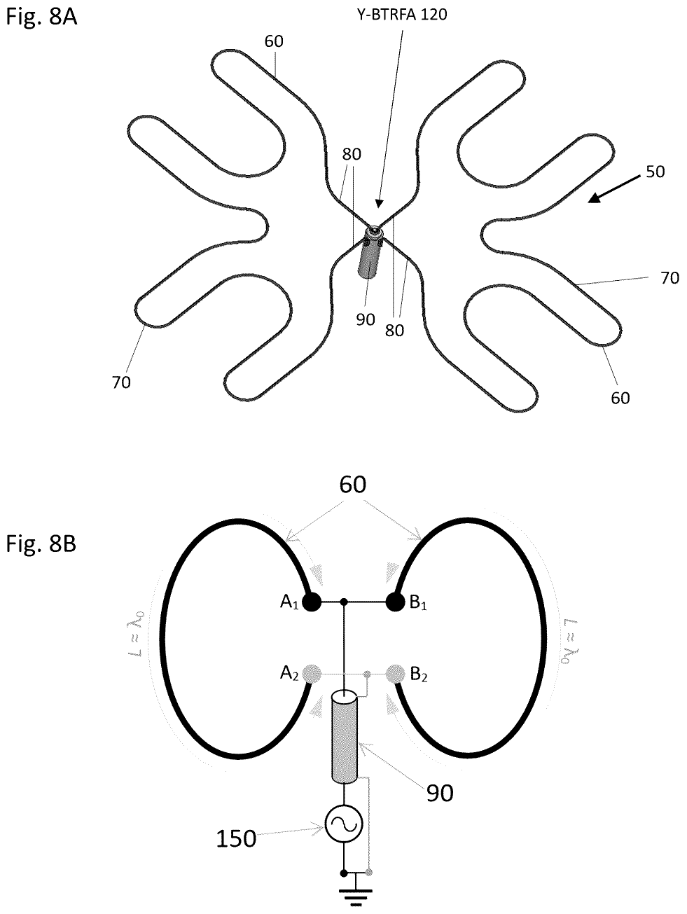

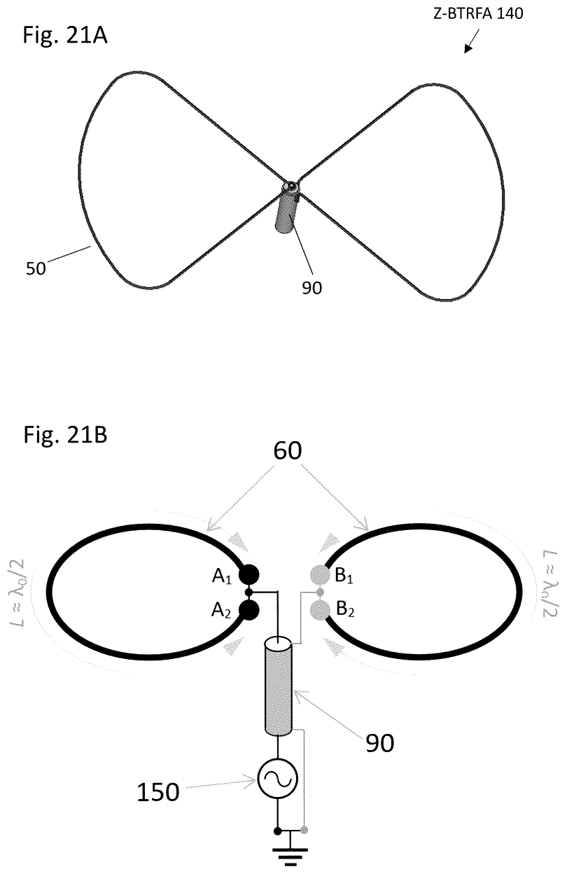

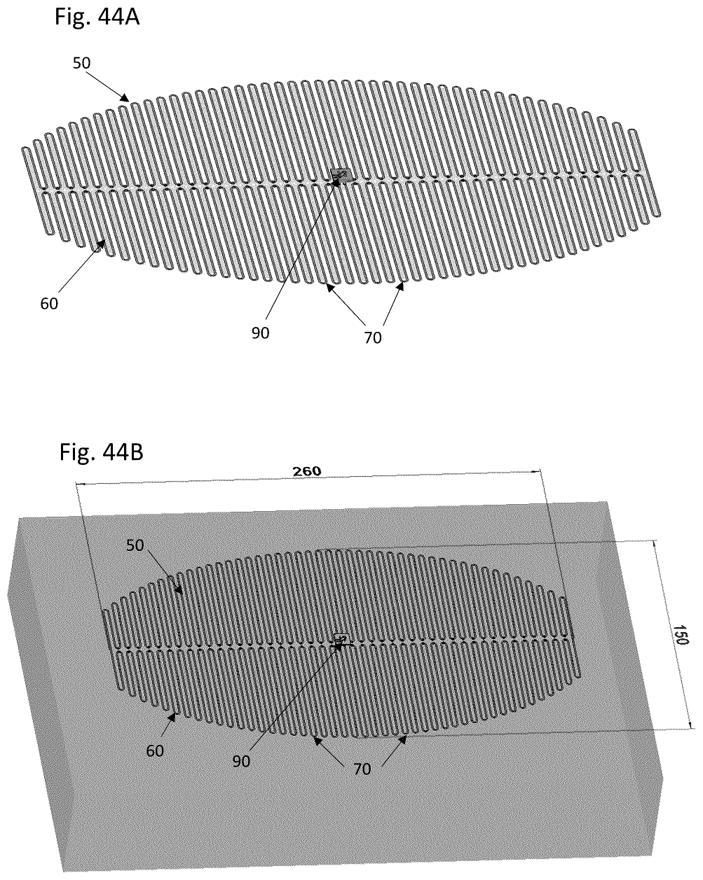

According to the present invention, the wire or PCB (60) within each within each of the at least two sections or halves (70) of the BTRFA (50) may be arranged in simple or complex meandering patterns, forming one or more geometrical shapes. These geometrical configurations can range from simple straightforward looped designs, as illustrated in FIG. 21, to intricate circumvoluted patterns, as depicted in FIGS. 1 and 8, or even highly complex, fully meandered circumvoluted structures, as shown in FIGS. 44, 45, and 47. The configurations may be spaced apart, as demonstrated in FIGS. 1 and 8, or densely packed with a high degree of meandering, e.g., fully meandered and compact configurations, as exemplified in FIGS. 44, 45, and 47. Furthermore, the arrangement of the at least two sections or halves (70) of the BTRFA (50) may be either symmetrical or asymmetrical. Preferably, the sections or halves (70) of the applicator (50) are symmetrically arranged. However, variations in the geometrical shapes, the number of sections or halves, their symmetry or asymmetry, and the degree of compactness may be implemented to optimize the design for different target regions or areas of human body tissue, ensuring adaptability to varying anatomical structures of the targeted body tissues.

According to a first embodiment of said arrangement of the wire or PCB structures (60), the at least two sections or halves (70) of the BTRFA (50) may be arranged to have an overall bow-tie shape, e.g., with a narrower central region with wider sections on either side. When the BTRFA (50) has such a bow-tie loop geometry, each section of half (70) of the BTRFA (50) comprises one loop are shown in FIG. 21. The bow-tie loop shaped BTRFA (50) thus comprises two looped structures that mirror each other along a central axis, each loop extending outward in a rounded or elliptical shape before returning toward the central region or junction. Alternatively, BTRFA (50) may have an overall multi-loop bow-tie shape, wherein each half comprises multiple circumvoluted loop structures arranged around the central axis. At the midpoint of the BTRFA (50), the circumvoluted loops converge at a central connection structure (90) forming a shared coupling connection.

According to a second embodiment of said arrangement of the wire or PCB structures (60), the at least two sections or halves (70) of the BTRFA (50) may be arranged to have a bow-tie complex butterfly shape. The bow-tie complex butterfly shaped BTRFA (50) has both a bow-tie shape (with a narrower central region with wider sections on either side) and a butterfly shape with at least two symmetrical or asymmetrical lobes or loops having a complex design with intricate geometrical patterns and varying contours. The bow-tie complex butterfly shaped BTRFA (50) thus comprises at least two sections or halves (70) that extend symmetrically or asymmetrically from a central axis. The structure exhibits a narrower central region transitioning into wider lobes or loops which may adopt meandering, looped, or interwoven geometrical patterns, for example resembling a butterfly shape, and forming either spaced or compact configurations. The two opposing lobes or loops of each section or half (70) of the BTRFA (50) may be flat or curved, facilitating adaptable placement against different anatomical body tissue surfaces. A central connector structure (90) couples the two sections or halves (70). The arrangement of these sections or halves (70) may be symmetrical or asymmetrical, depending on the application requirements and the anatomical body tissue region being targeted. The dimensions, curvature, and structural topology of the BTRFA (50) are adaptable to different tissue regions, allowing for precise targeting of RF energy.

According to a third embodiment of said arrangement of the wire or PCB structures (60), the at least two sections or halves (70) of the BTRFA (50) may be arranged to have highly compact meandered BTRFA (50) comprising at least two sections of halves (70) extending outwardly from the central connector structure (90). The BTRFA features parallel meandered highly compacted wire or PCB structure (60). Each section or half (70) comprises a series of meandering wire or PCB structures that are arranged in substantially parallel configurations. These meandered elements may be designed as looped, interwoven, or serpentine geometries forming a spaced or compact structure. The two sections or halves (70) may be symmetrical or asymmetrical, depending on application requirements and the anatomical region of the body tissue being treated. Additionally, the highly compact meandered configuration may have a symmetrical curvature allowing conformal placement over targeted body tissue, ensuring consistent and homogenous RF distribution.

The highly compact meandered BTRFAs according to this third embodiment preferably operate within VHF frequency ranges, namely 30-300 MHz, 30-200 MHz, 40-250 MHz, 50-200 MHz, 50-150 MHz, 100-150 MHz, 60-100 MHz, 50-70 MHz, 50-65 MHz, 60-65 MHz.

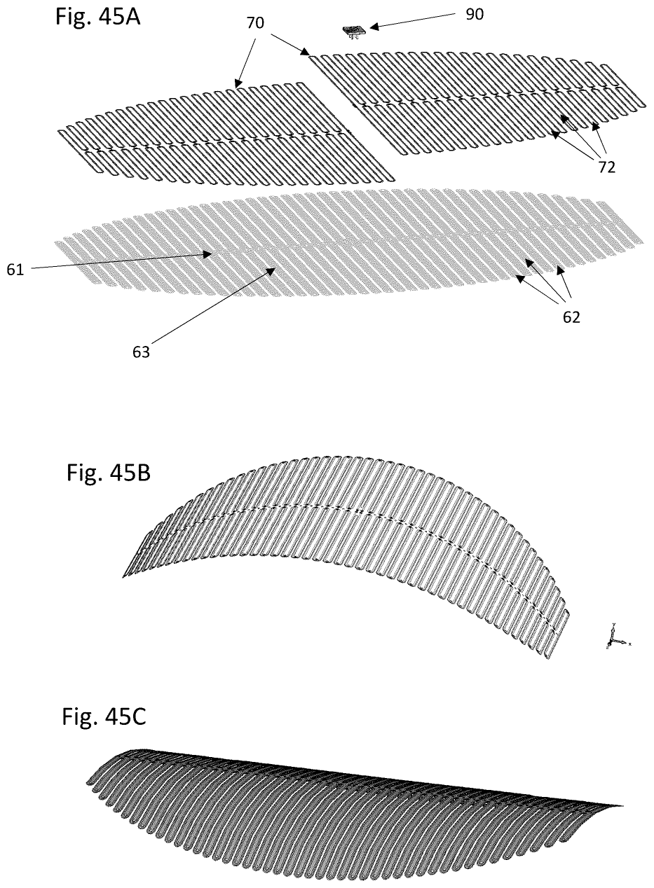

As illustrated in FIGS. 44 and 45, these BTRFAs are configured as fully compact meandered structure. Preferably, such large BTRFA may be fabricated made on a flexible printed circuit board (PCB) with a thin dielectric layer (63). The dielectric layer (63) serves as a structural substrate for the BTRFA architecture, which comprises a long elastic central strip (61) with several elastic branches (62) that are orthogonally attached to the central strip (61). The dielectric layer (63) may also serve as a base for two identical halves of conductive meandered transmission lines (70) that form the BTRFA structure. The conductive lines (72) forming the BTRFA comprises a thin layer, for example made of copper, which may have a width of less than 1 mm, for example approximately around 0.7 mm, and a thickness of less than 0.05 mm, for example approximately 0.035 mm. As depicted in FIG. 45B, the central elastic strip (61) enables the BTRFA structure to bend along its longitudinal axis, allowing it to conform to curved body tissue surfaces. Additionally, the elastic orthogonal branches (62) facilitate bending in a direction orthogonal to the primary longitudinal axis of the BTRFA, ensuring multidimensional flexibility. By combining these two bending capabilities the BTRFA can conform to complex anatomical structures, such as the curvature of the human head or any other targeted part of the body, maintaining a substantially uniform spacing for example of approximately 3 to 5 mm from the curved surface of the head or any other part of the targeted body.

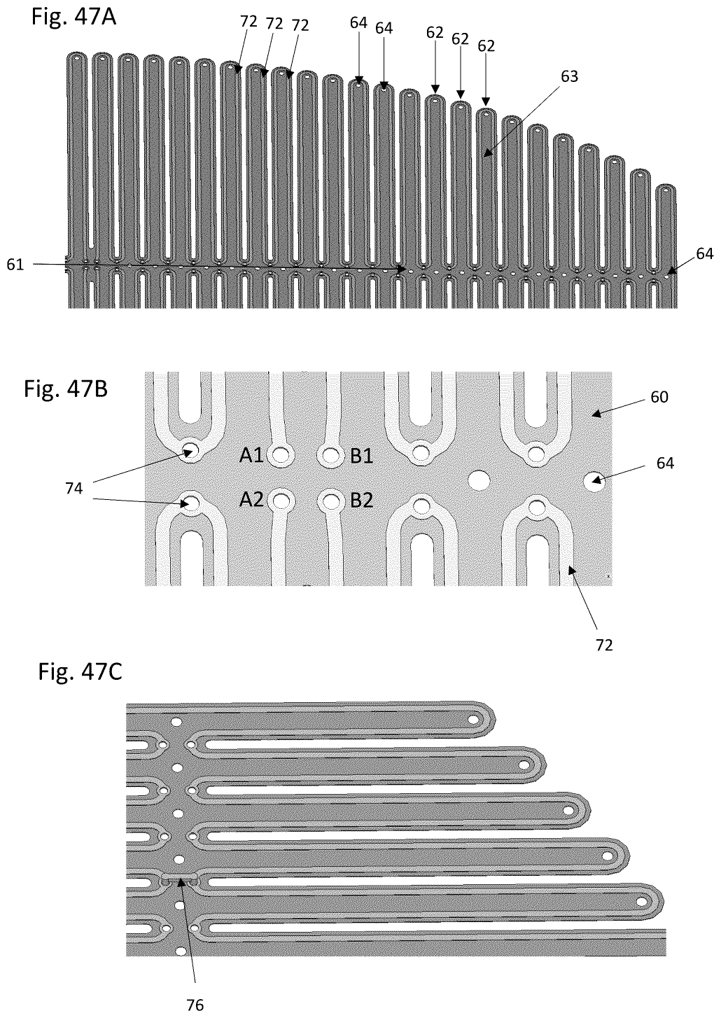

As illustrated in FIG. 47A, such large BTRFA according to this third embodiment may be fabricated on a flexible PCB with a thin dielectric layer (63). The thin dielectric layer (63) comprises a long elastic central strip (61) extending along the primary axis of the BTRFA, and multiple elastic branches (62) orthogonally attached to the central strip (61). The BTRFA structure incorporates conductive lines (72), which meander along the edges of the elastic branches (62). Additionally, the dielectric substrate (63) may comprise one or more means for attachment or mechanical vias (64) positioned at the extremities of the elastic branches (62), away from the central strip (61), and outside the conductive lines (72). These mechanical vias (64) serve as attachment points, allowing the BTRFA to be securely fixed to a housing structure, which may be composed of either fabric or lightweight plastic.

As illustrated in FIG. 47B, the central connection structure of the BTRFA may have four connection points (A1, A2, B1, and B2) to which the RF stimulatory signal is applied. The BTRFA may also comprise one or more metallized vias (74) positioned on the conductive lines (72) near the extremities of the elastic branches (62) adjacent to the central strip (61). As illustrated in FIG. 47C, the BTRFA may further comprise conductive shorting connectors (76) allowing to bridge corresponding pairs of metallized vias (74) facing to each other's (e.g., that are symmetrical positioned with respect to the center of the BTRFA), thereby allowing the total length of the BTRFA transmission line to be shortened and its operating frequency to be changed by appropriately connecting the corresponding pairs of vias. By selectively connecting symmetrically aligned metallized vias (74) using conductive shorting connectors (76), the total length of the BTRFA transmission line can be adjusted, allowing for modification of its operating frequency.

As indicated above, the wire or PCB (60) in each of these at least two sections or halves (70) is connected to a central connector structure (90), which may be a central coaxial connector structure (90), having a first conductor structure (100) or inner coaxial conductor (100) and a second conductor structure (110) or outer coaxial conductor (110), a first end (80) of each of these wires or PCBs (60) being connected to said inner coaxial or first conductor structure (100) and a second end (80) of each of these wires or PCBs (60) being connected to said outer coaxial or second conductor structure (110).

According to a first embodiment of the central connection structure of the BTRFA, the opposite ends (80) of the wire or PCB (60) of each half or section (70) being connected to the inner coaxial or first conductor structure (100) extend unbroken and preferably along a straight line across the central connector structure (90) from one half (70) to the other half (70). In other words, the first end (80) of the wire or PCB (60) of one half (70) and the second end (80) of the other half (70) are continuous connected across the central connector structure (90), forming a direct electrical path through the inner coaxial conductor (100). The other opposite ends (80) of the wire or PCB (60) of each half or section (70) being connected to the outer coaxial or second conductor structure (110), do not extend unbroken across the central connector structure (90) from one half (70) to the other half (70), but preferably form an interrupted straight line, meaning that the connection to the outer conductor (110) remains localized to each half (70) and does not establish a direct continuous path across the central connector (90).

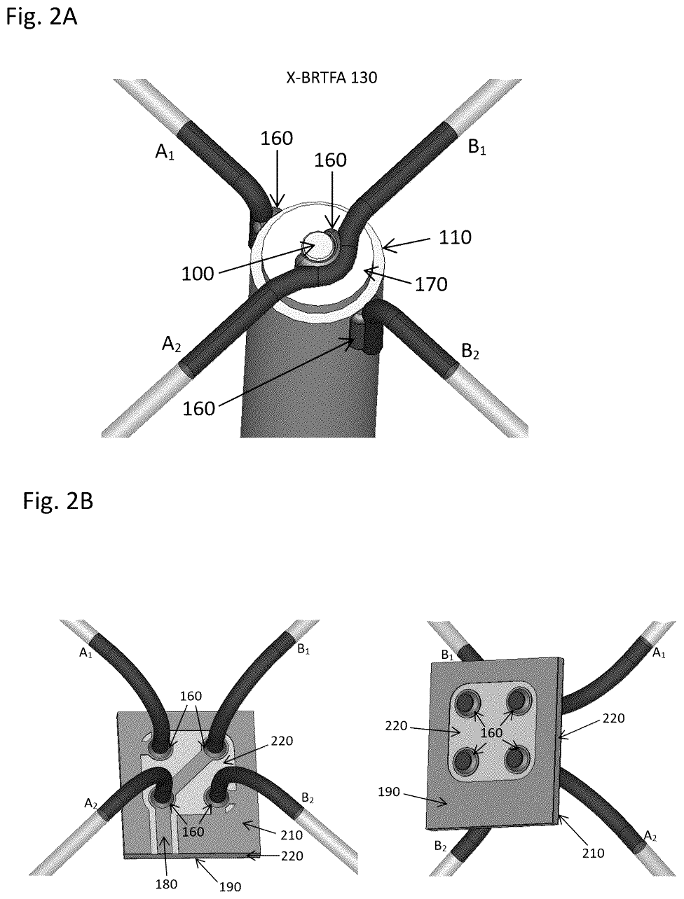

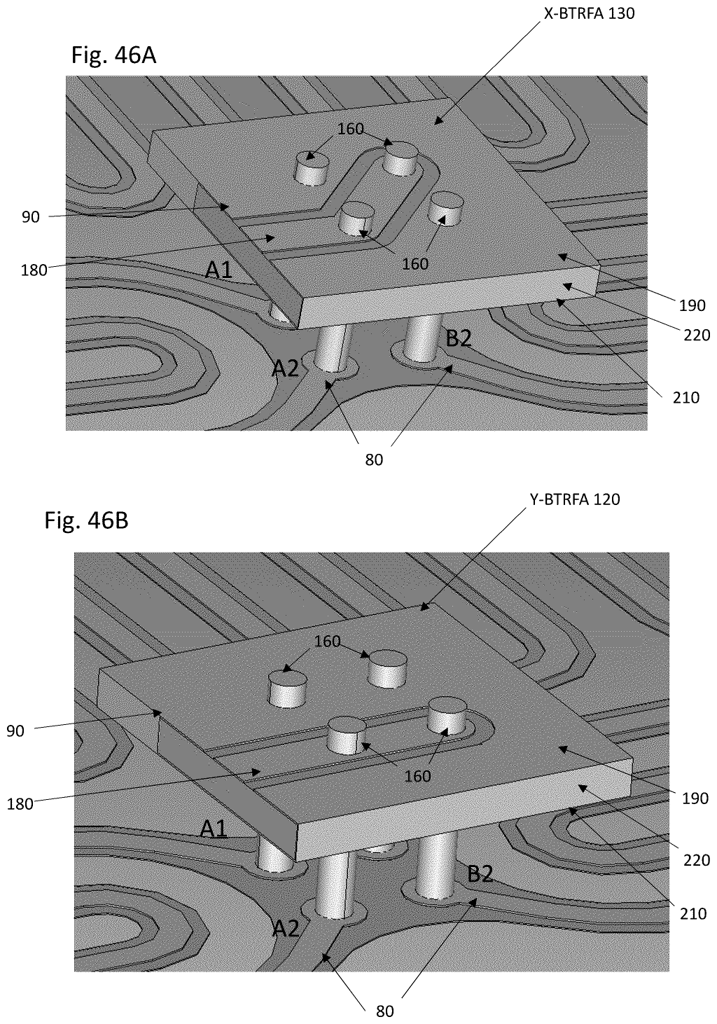

According to this first embodiment, the ends (80) of the wire or PCB (60) connected to the central connector structure (90) are arranged such that, when viewing from a top perspective onto the central connector structure (90), they are fully crisscrossed, forming an X-shaped configuration (130) as shown in FIGS. 1 and 2. FIG. 46A also illustrates a X-type PCB central connection.

A body tissue RF applicator (BTRFA) comprising this X-type central connection according to this first embodiment of the present invention is hereinafter referred as X-BTRFA (130).

FIG. 1B provides a schematic representation of the X-BTRFA (130), illustrating the electrical configuration and connectivity of its components. RF transmission line (180) connects the conductive loops with a generator of RF signal (150). RF transmission line (180) may be either coaxial cable, as illustrated in FIG. 1B, or printed transmission line (microstrip or some other type) as illustrated in FIG. 2B. The figure depicts a voltage source (150) supplying the RF signal to the applicator, the coaxial connector structure (90), which serves as the central connection point for the BTRFA, and endpoints A1, A2, B1, and B2 of the two wires or PCBs (80) in each of the two halves (70) of the wire structure (60), which are coupled to the coaxial connector structure (90). Conductive loops have connecting points marked as A1 and A2, for the first loop, and B1 and B2, for the second loop. Connection between points A2 and B1 may be connected to the central conductor (100) of the coaxial cable/transmission line (90) and further to the output of the RF generator (150). Connection points A1 and B2 may be both connected to the outer conductor (110) of the coaxial cable (90) and further connected to the electrical ground, which is also the reference ground for RF generator (150).

This crossover connection between points A1 to B2 and A2 to B1 in FIGS. 1, 2, and 46A resembles the shape of letter X and therefore this type of BTRFA is named X-type BTRFA (130).

FIG. 2A provides a detailed view of the X-BTRFA (130) as illustrated in FIGS. 1A and 46A, with the endpoints A1, A2, B1, and B2 of the wires or PCBs (80), the central coaxial connector structure (90), which links the two halves (70) of the BTRFA, the inner coaxial conductor (100) and outer coaxial conductor (110) within the coaxial connector (90), an insulator (170) positioned between the inner and outer coaxial conductors (100, 110) to maintain electrical isolation, and solder or connection points (160), marking the electrical junctions where the applicator wires or PCB traces are connected to the coaxial connector structure (90).

FIGS. 2B and 46A provide a detailed view of the X-BRTFA (130), depicting its connection to a printed circuit board (PCB) as an alternative embodiment to the configuration shown in FIG. 2A. These figures illustrate both a top view (left side), showing the primary structural elements, and a bottom view (right side), revealing additional grounding and dielectric components. In this PCB-integrated configuration, the endpoints A1, A2, B1, and B2 of the two wires or PCB traces (80) in each of the two halves (70) are electrically coupled to either with an RF input microstrip line (180), or a top ground (GND) layer (190) at the connection points (160). Additionally, the structure includes a bottom ground (GND) layer (210), a dielectric substrate (220), which separates the top conductive layers from the bottom GND layer (210), ensuring electrical isolation and controlled impedance.

FIG. 1A presents a complete view of the X-BRTFA (130), as shown in FIG. 1B, with a coaxial connector structure (90) configured either according to FIG. 2A, featuring a direct coaxial feed, or according to FIGS. 2B and 46A, incorporating a PCB-based connection with a meandered wire or PCB applicator structure.

According to a second embodiment of the central connection structure of the BTRFA, the first ends (80) of the wire or PCB (60) of each section of half (70) may be connected to the inner coaxial or first conductor structure (100) extend unbroken along a Y-shaped line or path from one half (70) to the other half (70). Conversely, the second ends (80) of the wires or PCBs (60) of each section of half (70) may be connected to the outer coaxial or second conductor structure (110) thereby forming a Y-shaped line or path that is interrupted by the central connector structure (90).

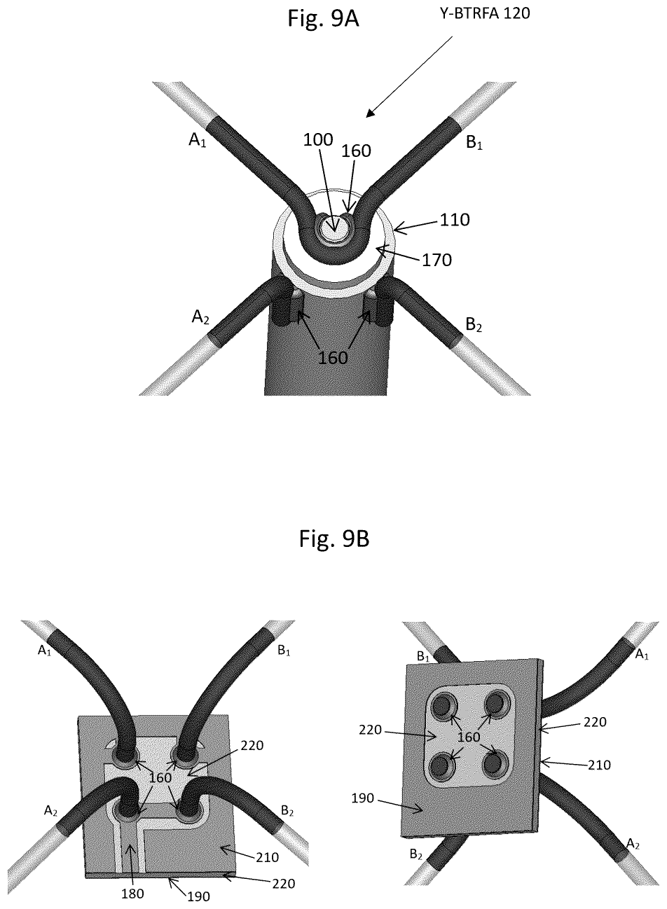

When viewed from a top-down perspective onto the central connector structure (90), the ends (80) of the wires or PCBs (60) connected to the central connector structure (90), appear half-crisscrossed, with each crisscrossed half forming a Y-shaped pattern, as shown in FIGS. 8 and 9. FIG. 46B also illustrates a Y-type PCB central connection.

A body tissue RF applicator (BTRFA) comprising this Y-type central connection BTRFA according to this second embodiment of the present invention is hereinafter referred as “Y-BTRFA” (120).

FIG. 8B provides a schematic representation of the Y-BTRFA (120), illustrating the electrical configuration and connectivity of its components. The wire structures (60) are shown only schematic and simplified as two loops. RF transmission line (180) connects the conductive loops with a generator of RF signal (150). RF transmission line (180) may be either coaxial cable, as illustrated in FIG. 8B, or printed transmission line (microstrip or some other type) as illustrated in FIG. 9B. The figure depicts a voltage source (150) supplying the RF signal to the applicator, the coaxial connector structure (90), which serves as the central connection point for the BTRFA, and endpoints A1, A2, B1, and B2 of the two wires or PCBs (80) in each of the two halves (70) of the wire structure (60), which are coupled to the coaxial connector structure (90). Conductive loops have connecting points marked as A1 and A2, for the first loop, and B1 and B2, for the second loop. As shown in FIGS. 8, 9, and 46B, connection points A1 and B1 may be connected to the central conductor (100) of the coaxial cable/transmission line (90) and further to the output of the RF generator (150). Also, points A2 and B2 are both connected to the outer conductor (110) of the coaxial cable (90) and further connected to the electrical ground, which is also the reference ground for RF generator (150).

This connection between points A1 to B1 and A2 to B2 in practical realization of BTRFA connection to coaxial cable resembles the shape of letter Y and therefore this type of BTRFA is named Y-type BTRFA (120).

Additionally, FIGS. 9A and 46B provides a detailed view of the Y-BTRFA (120) as illustrated in FIGS. 8A, with the endpoints A1, A2, B1, and B2 of the wires or PCBs (80), the central coaxial connector structure (90), which links the two halves (70) of the BTRFA, the inner coaxial conductor (100) and outer coaxial conductor (110) within the coaxial connector (90), an insulator (170) positioned between the inner and outer coaxial conductors (100, 110) to maintain electrical isolation, and solder or connection points (160), marking the electrical junctions where the applicator wires or PCB traces are connected to the coaxial connector structure (90).

FIGS. 9B and 46B provide a detailed view of the X-BRTFA (130), depicting its connection to a printed circuit board (PCB) as an alternative embodiment to the configuration shown in FIG. 9A. These figures illustrate both a top view (left side), showing the primary structural elements, and a bottom view (right side), revealing additional grounding and dielectric components. In this PCB-integrated configuration, the endpoints A1, A2, B1, and B2 of the two wires or PCB traces (80) in each of the two halves (70) are electrically coupled to either with an RF input microstrip line (180), or a top ground (GND) layer (190) at the connection points (160). Additionally, the structure includes a bottom ground (GND) layer (210), a dielectric substrate (220), which separates the top conductive layers from the bottom GND layer (210).

FIG. 8A presents a complete view of the Y-BTRFA 120 as shown in FIG. 1B, with a coaxial connector structure (90) configured either according to FIG. 9A featuring a direct coaxial feed, or according to FIGS. 9B and 46B incorporating a PCB-based connection with a meandered wire or PCB applicator structure.

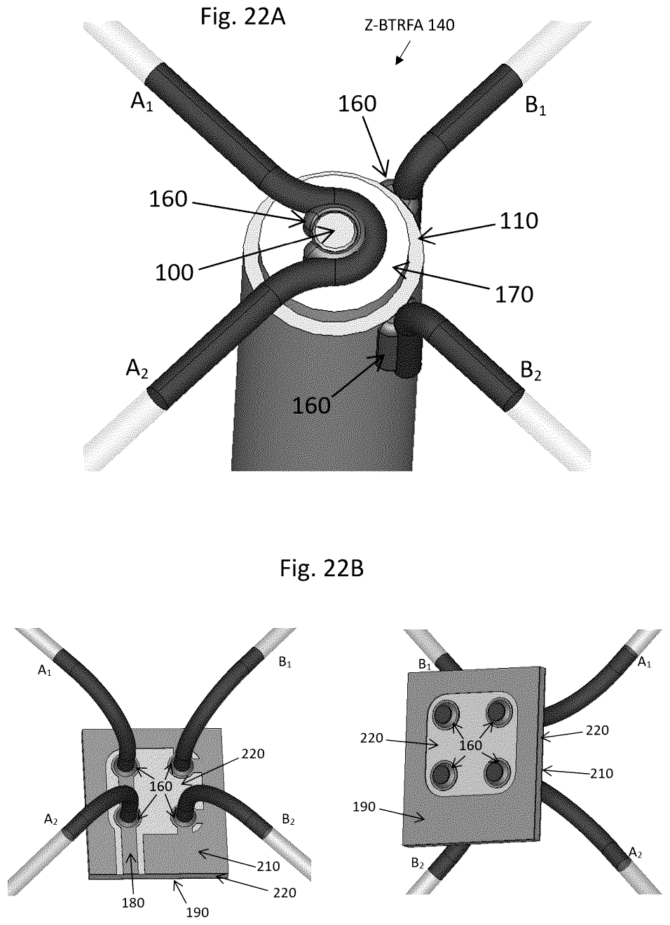

According to a third embodiment of the central connection structure of the BTRFA, the ends (80) of the wire or PCB (60) of each section or half (70), upon connection to the central connector structure (90), form a structure as illustrated in FIGS. 21 and 22. In this configuration, the ends (80) of the wire or PCB (60) in one half (70) are connected to the inner coaxial or first conductor structure (100) and the ends (80) of the wire or PCB (60) of the opposite or other half (70) are connected to the outer coaxial or second conductor structure (110).

In the Z-type central connection structure, the ends (80) of the wires or PCBs (60) connected to the outer coaxial or second conductor structure (110) form a V-shaped configuration or line. Similarly, the ends (80) of the wire or PCB (60) connected to the inner coaxial or first conductor structure (100) also form a V-shaped configuration or line.

Unlike the Y-BTRFA (120), in the Z-BTRFA (140), both ends (80) of the wire or PCB (60) in one half (70) of the wire or PCB structure (60) are connected to the inner coaxial or first structure (100), whereas both ends (80) of the wire or PCB (60) in the other half (70) are connected to the outer coaxial or second structure (110).

A body tissue RF applicator (BTRFA) comprising this Z-type central connection BTRFA according to this third embodiment of the present invention is hereinafter referred as “Z-BTRFA” (140).

FIG. 21B shows a further schematic representation of the Z-BTRFA (140), depicting the Z-type connection between the coaxial conductors and the central antenna wires between the coaxial conductors and the central antenna wires. The wire or PCB structure (60) with the applicator wires is illustrated in a simplified schematic form, represented as two loops for clarity. FIG. 21B also includes a voltage source (150) and the central coaxial connector structure (90). It also shows connection endpoints (A1, A2, B1 and B2) in each of the two halves (70) that are connected to the coaxial connector structure (90). FIG. 22A provides a detailed view of the Z-BTRFA (140) as shown in FIG. 21A, illustrating the endpoints A1, A2, B1 and B2 of the wires, the central coaxial connector structure (90), the inner coaxial conductor (100), the outer coaxial conductor (110), an insulator (170) positioned between the inner and outer conductors, and solder or connection points (160).

FIG. 22B illustrates a Z-type connection in a PCB-integrated design of the Z-BTRFA (140), providing a top detailed view (left side) and a bottom detailed view (right side), showing how the endpoints (A1, A2, B1, and B2) of the two PCBs (80) in each of the two halves (70) are connected to an RF input microstrip line (180) or to a top ground (GND) layer (190) at the connection points (160). The Figure also depicts a bottom ground (GND) layer (210) and a dielectric substrate (220) which serves as the supporting PCB material. FIG. 21A presents a complete view of the Z-BTRFA (140) as depicted in FIG. 21B, showing a coaxial connector structure (90) configured according to FIG. 22 and with simple looped antenna wire configuration.

According to a fourth embodiment of the central connection structure, BTRFA may be constructed using PCB and may comprise a central coaxial connector switch (90). The switching mechanism enables the transition between an X-type connection (X-BTRFA 130) and a Y-type connection (Y-BTRFA 120) without requiring the replacement of the entire BTRFA. By integrating an RF switch into the output circuit of the transmitting RF amplifier, it becomes possible to selectively establish electrical connections with the feed points A1, A2, B1, and B2. These feed points can alternately exhibit the characteristics of both the X-type and Y-type feeding modes of the BTRFA, thereby modifying the electromagnetic field distribution characteristics. The selection of the feeding mode may be controlled via an additional control signal, which governs the output states of the RF switch, allowing the selection between X-type or Y-type feeding modes, or the rapid switching between the two states, enabling dynamic reconfiguration of the applicator. By combining the X and Y feeding configurations, the BTRFA achieves a more uniform and widespread SAR (Specific Absorption Rate) distribution within the targeted biological tissue, optimizing the RF energy deposition.

Both PCB-based connections may comprise an RF input microstrip line (180), a first electrically conductive layer (190), a second electrically conductive layer (210) and a dielectric layer (220) positioned between the first and second electrically conductive layer (190, 210). Endpoints A1, A2, B1 and B2 of the two PCBs or wires (80) in each of the two halves (70) are connected to either an RF input microstrip line (180) or to a top ground (GND) layer (190) at the connection points (160).

BTRFA (50) may be constructed from wire or printed on a PCB structures. BTRFA may be realized as flexible PCB integrated with PCB or like old fashion wire structures, sufficiently flexible and attached to a PCB.

When BTRFA are made of wire structures, they may be made of metal wire, preferably consisting of some low loss metal, like copper, usually with round (circular) cross section, or any other suitable antenna wire. The metal must be sufficiently flexible for generating complex bowtie butterfly shapes as described above. Optimal thickness of wires may easily be determined by a skilled person in the art. For practical mechanical realizations the thinner is better, but it increases metal losses and therefore decreases the overall efficiency. Wired BTRFA preferably have a wire diameter in a range from 0.1 mm and 1 mm, or between 0.2 mm and 1 mm, or between 0.3 mm to 1 mm. Most preferred wire diameter may be between 0.5 mm and 0.9 mm. BTRFAs with a very thin wires with dimensions 0.1 mm×0.2 mm were also made and tested. Besides a slight increase of losses in metal, the reduction of the wire thickness provided similar characteristics of the BTRFA, thereby showing that BTRFA provided the desired characteristics even with a very thin wire or printed transmission line as long as necessary mechanical rigidity was maintained. The wire cross section may be round, or rectangular (0.1 mm×0.8 mm), or square (0.5 mm×0.5 mm). At frequencies below 3 GHz, these rectangular cross sections may be equivalent to the circular cross sections of the same area size.

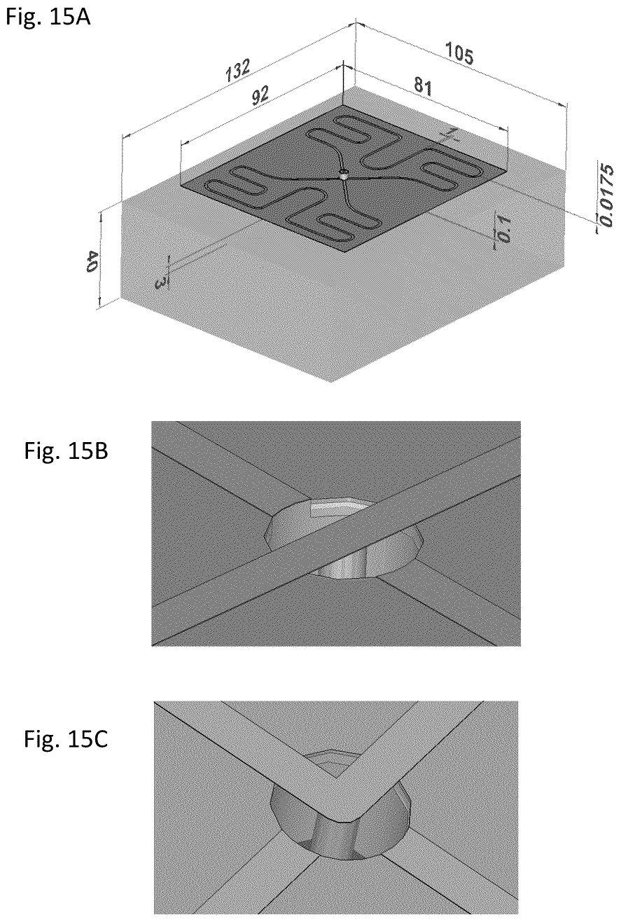

Alternatively, BTRFA may be made of printed transmission lines, which are printed on thin metal strips on a thin dielectric substrate that provides mechanical support. In particular, both Y and X versions of BTRFA according to the present invention may be realized as a printed structure on very thin and flexible dielectric substrate. Optimal thickness or widths of the printed transmission lines can be easily determined by a skilled person in the art. By way of example, thickness of the thin metal strips may be at least 0.01 mm and the width may be at least 0.5 mm. The metal thickness may be around 0.035 mm or reduced to 0.0175 mm or even lower if the appropriate flexy PCB technology is used, while the dielectric thickness may be around 0.1 mm or even thinner (FIG. 15A). The PCB is preferably printed on substrate which may be easily curved or bent, such as any casting foil, and is called “flexy PCB”. Therefore, using such printed transmission lines, the BTRFA can be realized on flexy PCBs and integrated with rigid PCBs containing active electronic components of the device.

BTRFA made of PCB structures may have identical dimensions and shapes as that of BTRFA made of wires as described above. Also, printed BTRFA were placed at the same distance from body tissue sample with the side with printed metal facing toward the tissue. The connections between the coaxial cable and printed PCB metal trace for the X connection (FIG. 15B and FIG. 46A) and the Y connection (FIG. 15C, right side and FIG. 46B).

Preferred BTRFA are made of curved wire structures or of flexible or flexy PCB structure to facilitate positioning adjacent to or in proximity to human tissue.

FIG. 1B and FIG. 8B show two versions of BTRFA, X-BTRFA and Y-BTRFA, respectively. Each version consists of two conductive wire or PCB loops. The length of each of the conductive wire or PCB loops is preferably approximately λ₀ (at f_{c}), which is the free space wavelength of the RF signal delivered to body tissue, at its operating central frequency (f_{c}). FIGS. 21 and 22 show Z-BTRFA, wherein the bow-tie butterfly shape BTRFA have two time smaller loop lengths (λ₀/2@f_{c}—half of the wavelength at the intended working frequency), unlike X-BTRFA and Y-BTRFA that have two times larger loop lengths of λ₀@f_{c} and central connection to the input transmission line or coaxial cable are referred hereinafter as Z-BTRFA.

The operating frequency of BTRFA applicator depends on the BTRFA size which is related to the total wire length in each of two antenna branches. For Y-BTRFA (120) the total wire length as well as the overall wire shape is identical for X-BTRFA 130, which gives wire length in each branch of approximately one wavelength (at the desired operating frequency) which is about 320 mm at 915 MHz, about 3000 mm at around 100 MHz, and about 6000 mm at around 50 MHz. The resonant frequency of such antenna can be changed if antenna is placed close to a large dielectric object having high relative dielectric constant (relative dielectric permittivity), which is a body tissue typically between 45 and 55.

The major function of the BTRFA, and an important aspect of the invention, is to efficiently deliver RF energy into a body tissue. “Efficiently deliver” includes several tasks besides power efficiency. It is desirable to have RF energy spread evenly over sufficiently large areas to cover, for example, the entire surface of a human head with reasonable small number of the BTRFAs. BTRFA should also be able to adjust its shape to accommodate the curvature of a body tissue, for example of the human head without compromising its power efficiency. It is also desirable to minimize the RF energy emission into a free space for easier fulfilling all existing electromagnetic compatibility (EMC) requirements and regulations.

Body tissue radiofrequency applicators or antennas according to the present invention may have variable shapes and sizes adaptable to any part of human body tissues and to obtain specific coverage area and depth of exposure to stimulatory signals for which treatment is desired.

When head treatment is desired, the BTRFA should be suitable to the various human sizes, to the positioning onto the head, to the brain areas targeted and types of neurodegenerative diseases. For example, BTRFA working at 915 MHz sizes may range from (20 mm to 300 mm)×(20 mm to 300 mm). Small size BTRFA may be positioned for example on the top of the head from ear to ear, thereby covering for example the temporal and parietal lobes of a brain subject. According to another preferred embodiment, BTRFA working at 50-100 MHz may be 10 times larger compared to BTRFA working at 915 MHz, so that a single BTRFA may be designed so as to cover the entire head of the patient.

The largest dimension or length of the BTRFA is determined by the operating frequency which is approximately half wavelength including the influence of the adjacent body tissue. Therefore, when operating at around 915 MHz, the wire or PCB length of the RF applicator according to the present invention may be between 200 and 800 mm, preferably around 600 mm. According to another preferred embodiment, the BTRFAs according to the present invention operating at frequency band ranging from 50 MHz to 100 MHz and thus has a total wire or PCB length in a range of 2000 to 8000 mm, or 3000 to 7000 mm, or around 3000 mm for BTRFA 100 MHz and 6000 mm for BTRFA 50 Mhz.

In addition to being suitable for providing an efficient and reliable zone of electromagnetic exposure, they also provide a reliable, reproducible, and efficient specific absorption rate (SAR) within the treated or targeted human brain tissue. SAR is the measure of the rate of energy which is absorbed per unit of mass of a human body when exposed to a radiofrequency electromagnetic field and is expressed in watts per kilogram (W/kg). Preferred SAR values according to the present invention range from 0.5 to 3 W/kg. Most preferably SAR values are from 1 to 2 W/kg or from 1.5 to 2 W/kg such as 1.5 W/kg or about 1.5 W/kg or such as 2 W/kg or about 2 W/kg.

BTRFAs according to the above preferred embodiments, e.g., X-BTRFAs (130), Y-BTRFAs (120) or Z-BTRFAs (140) may have different efficiency and Specific Absorption Rate (SAR) within the human body tissue. Topologically, X-BTRFA (130) has central symmetry while Y-BTRFA (120) and Z-BTRFA (140) have axial symmetry.

X-BTRFA (130) and Y-BTRFA (120) according to the above embodiments of the present invention have high performance or efficiency when placed adjacent to human body tissue, which are dielectric objects having large dielectric constant with specific conductivity. In particular, BTRFAs having either X or Y types of connections have been shown to deliver between 85% and 92% of radiofrequency energy available from the generator to the adjacent body tissue. In addition, BTRFA can deliver a large amount of radiofrequency energy to a large area and volume of the body tissue directly exposed to the BTRFAs, with a high SAR value up to around 10 W/kg or up to 18 W/kg.

X-BTRFA (130) delivered radiofrequency energy within a large volume of a body tissue with maximum SAR value at four spots symmetrically placed around the connector. The tissue closest to connector received relatively low SAR value. X-BTRFA (130) achieved a SAR value up to 10.8 W/kg. X-BTRFA (130) radiated only about 0.8% of radiofrequency energy into a free space. This is a significant characteristic that could eliminate additional shielding for achieving the required EMC standards.

Y-BTRFA (120) also delivered radiofrequency energy within a large volume of a body tissue similar to the X-BTRFA (130), but with maximum SAR value close to central connector structure. Y-BTRFA (120) achieved a SAR value up to 18.9 W/kg. Y-BTRFA (120) radiated about 6.2% of radiofrequency energy into a free space which is also very satisfying.

When compared to the X-BTRFA (130) and Y-BTRFA (120), Z-BTRFA (140) delivered radiofrequency energy within a smaller volume of a body tissue but with the highest maximum SAR value up to 23.7 W/kg, close to connector. Z-BTRFA (140) radiated about 5.75% of radiofrequency energy into a free space which is comparable to the amount radiated by Y-BTRFA (120) (6.12%), but much larger than in case of X-BTRFA (130) (0.8%).

Considering all the presented characteristics of the X-BTRFA (130), Y-BTRFA (120), and Z-BTRFA (140), each of the showcased types can find its application in devices used for efficient and controlled delivery of electromagnetic wave energy to various bodily tissues. X-BTRFA (130) provides a uniform distribution over a broader tissue volume with very low radiation losses, meaning the smallest portion of electromagnetic energy that is radiated into the surrounding space. Y-BTRFA (120) provides a greater depth of penetration of electromagnetic waves into living tissue, while maintaining an unchanged coverage width and slightly higher levels of SAR. Z-BTRFA (140) may achieve a maximum depth of penetration with the highest SAR value in significantly smaller volume and surface area of tissue.

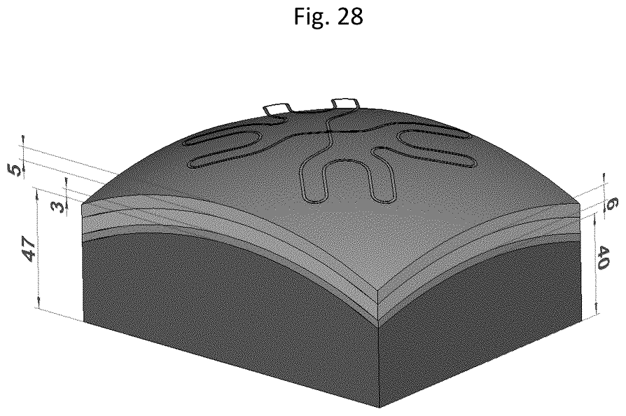

Applicators, namely Y-BTRFA (120), X-BTRFA (130) or Z-BTRFA 140 according to the present invention, being either wired or PCB, may be flat, flexible, or curved to adapt to the form of the targeted body tissue, such as for example the head or skull of the patient. The total length of the wire is extended due to replacing the homogeneous tissue model with multi-layer model (with various dielectric constant and conductivity for each layer). By way of example, FIG. 28A shows a curved BTRFA model above a curved multi-layer tissue sample such a human head.

SAR distributions have been analyzed for X-BTRFA (130) and Y-BTRFA (120) operating at 915 MHz with limited SAR values of 1 W/kg, 2 W/kg, 2.5 W/kg, 5 W/kg, with a generator having a power of 1 W and placed at 3 mm of the head surface.

Results for SAR values of 1 W/kg and 2 W/kg showed that X-BTRFA (130) provided more spread and scattered SAR distribution, while Y-BTRFA (120) gives more centered SAR distribution. For SAR values greater than 5 W/kg, X-BTRFA (130) provided a few small hotspots that are spread away from the center, while Y-BTRFA (120) provided a significantly larger volume beneath the center of the applicator. The maximum SAR (1 g) value for X-BTRFA (130) is 5.89 W/kg and for Y-BTRFA (120) is 8.6 W/kg (data not shown), which means that X-BTRFA (130) distributed RF energy more evenly with smaller and less intense. SAR values were obtained only during pulse duration; the total energy delivered would be reduced depending on the repetition rate of the signal.

Such distinctive characteristics provided more flexibility since both applicators could be used separately and in combination, thereby allowing them to obtain specific and desired SAR distributions to a specific area of the treated human tissue. In particular, both X-BTRFA (130) and Y-BTRFA (120) may be used in combination within the same medical device, such as a transcranial medical device in order to adapt the radiofrequency exposure and SAR to specific parts of the brain.

BTRFA (50) according to any of the embodiments described above may advantageously be combined with one or more low-lever lasers and/or light-emitting diodes (LEDs) (200) configured to emit in the red and/or near-infrared (NIR) spectrum. The light sources may include LEDs (200) or a low-level laser source, generating one or more signals within the red and NIR spectral range. Preferably, one or more LEDs—or an array of LEDs—emitting red and/or NIR signals may be embedded or integrated within the structural framework of the BTRFA (50). According to the present invention, the integration of LEDs within the BTRFA enables the synergistic administration of pulsed electromagnetic signals combined with red and NIR light to body tissue in subjects in need of such treatment.

The one or more LEDs (200) are configured to emit red and/or NIR signals. The red signals may comprise one or more wavelengths ranging from 620 nm to 680 nm, preferably at peak wavelengths of approximately 630 nm, 660 nm, or 670 nm. The NIR signals may comprise one or more wavelengths within the 800 nm to 1100 nm range, with preferred peak wavelengths around 810 nm, 830 nm, 880 nm, and/or within the range of 1060 nm to 1070 nm. The red and NIR signals are preferably pulsed, either at the same or different repetition rates, typically within the range of 10 Hz to 100 Hz, preferably 10 Hz to 60 Hz, or at approximately 20 Hz or 40 Hz.

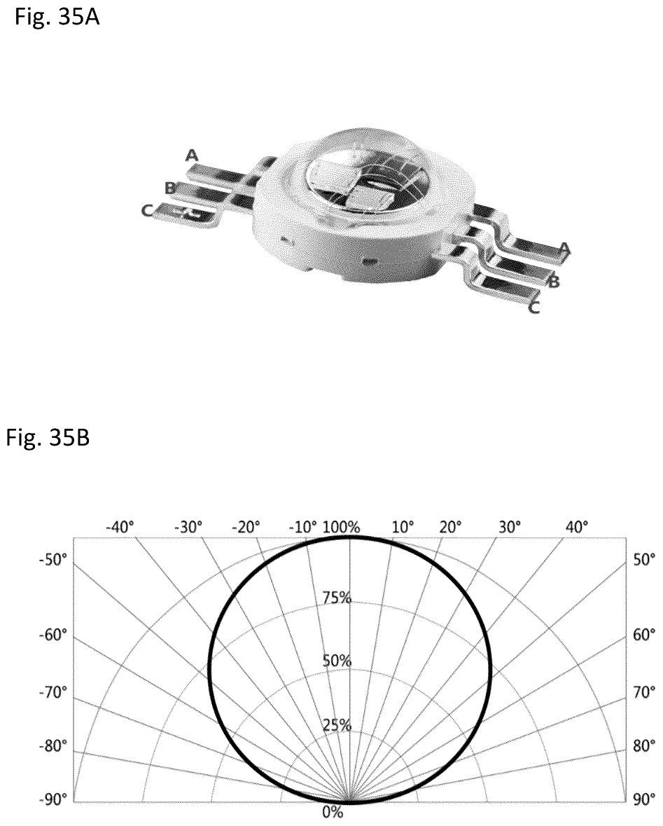

Preferred LEDs (200) may be 3-in-1 LEDs with three independent chips, each emitting at different red and NIR wavelengths, such as 660 nm, 810 nm, and 1064 nm. FIG. 35 illustrates an example of custom-made 3-in-1 LEDs (200) which may have the following specifications (i) 660-665 nm chip: 2.0-2.4 V, 20-30 lumens, 350 mA; (ii) 805-810 nm chip: 2.0-2.4 V, 100-200 mW, 350 mA; and (iii) 1050-1070 nm chip: 2.0-2.4 V, 100-200 mW, 350 mA.

The BTRFA-LED combination may be positioned in proximity to a body tissue, such as the head, to directly emit pulsed electromagnetic signals along with red and NIR light comprising wavelengths of approximately 630 nm, 660 nm, or 670 nm, in combination with NIR signals ranging from 808 nm to 880 nm and 1060 nm to 1070 nm, may be directed toward the brain.

LEDs (200) used in combination with the BTRFAs (50) operate within a power range of 25 mW to 1 W, and preferably around 50 mW to 500 mW, depending on the selected repetition frequency and duty cycle of the LEDs (200). By way of example, a 3-in-1 LED having an average power of 455 mW, a pulsed signal with duty cycle of 0.125 gives equivalent total power of about 57 mW, and it could be adjusted to some other values by changing duty cycle value, for example it could be tuned to an average total power of 100 mW by adjusting the duty cycle.

The beam spot sizes of LEDs (200) may range from 0.1 cm² to 1 cm², with a preferred power density (irradiance) between 10 mW/cm² and 100 mW/cm², more preferably around 50 mW/cm². Each LED (200) preferably delivers energy in the range of 1 to 50 Joules (J), more preferably around 25 J to 30 J per LED. The fluence (energy density per unit area) per LED (200) is preferably within 5 J/cm² to 200 J/cm², more preferably around 100 J/cm². The total dose per treatment session, calculated as the fluence per LED multiplied by the number of LEDs, preferably falls within 2000 J/cm² to 7000 J/cm², more preferably between 3000 J/cm² and 6000 J/cm², and even more preferably around 5000 J/cm².

The BTRFA-LED combination integrates BTRFA (50) emitting pulsed electromagnetic waves with LEDs (200) emitting red and/or NIR light. Each BTRFA (50) unit preferably includes between 2 and 12 LEDs, or 2 to 10 LEDs, or 4 and 8 LEDs, or around 5-6 LEDs per unit.

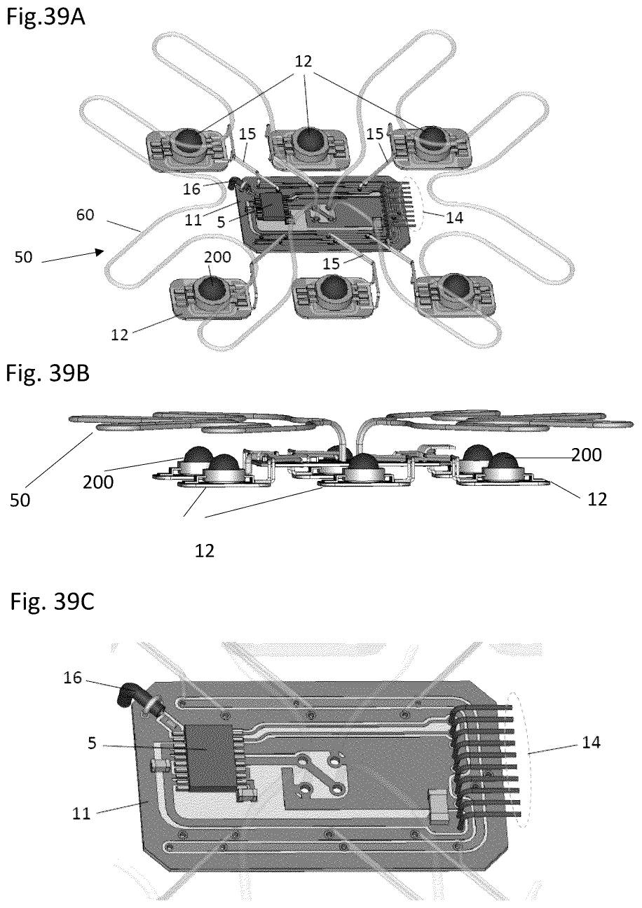

By way of example, FIG. 39 illustrates a BTRFA (50) integrated with six LEDs (200). This shows an example on how the LEDs may be embedded within the BTRFA structure, ensuring optimal placement for delivering pulsed electromagnetic signals and red/NIR light to the intended treatment area.

The red and NIR LED diodes may operate continuously or in alternating pulses overlapping with the pulsed electromagnetic wave emission from the BTRFA.

The cycle of repetition or pulsation of the red and NIR LEDs may involve a 40 Hz repetition frequency with a 50% duty cycle, where each pulse lasts 12.5 ms, followed by a 12.5 ms pause, leading to a total LED power consumption and heat dissipation (PC-HD) of 50%. Alternatively, a 20 Hz repetition frequency with a 50% duty cycle may be used, involving 25 ms pulses followed by 25 ms pauses. Such a “wavelength-sequential” LED sequence may comprise one LED at a specific wavelength activated for 25 ms, followed by a 25 ms pause, while all other LEDs remain OFF. The repetition frequency is 10 Hz, with an LED duty cycle of 25%.

Alternatively, a variation without pauses may be used, where one wavelength is active for 12.5 ms, leading to a 26.67 Hz frequency with a 33.33% duty cycle. There may be a complete pause between each LED emission/activation sequence. The total LED power consumption and heat dissipation (PC-HD) is 18.75% of the maximum. Possible alternative of the “wavelength-sequential” LED sequence may be without a pause in between each LED emission/activation sequence. One wavelength is active at a time for 12.5 ms. Repetition frequency for such sequence is 26.666 Hz, with duty cycle of each LED of 33.33%. The total LED power consumption and heat dissipation (PC-HD) is 33.33% of the maximum. In addition, it is possible to have variations of the duration of the single LED pulse, all having the same duty cycle of each LED of 33.33%, PC-HD equal to 33.33% of the maximum, and all BTRFA operating under equal alternating “LED conditions”.

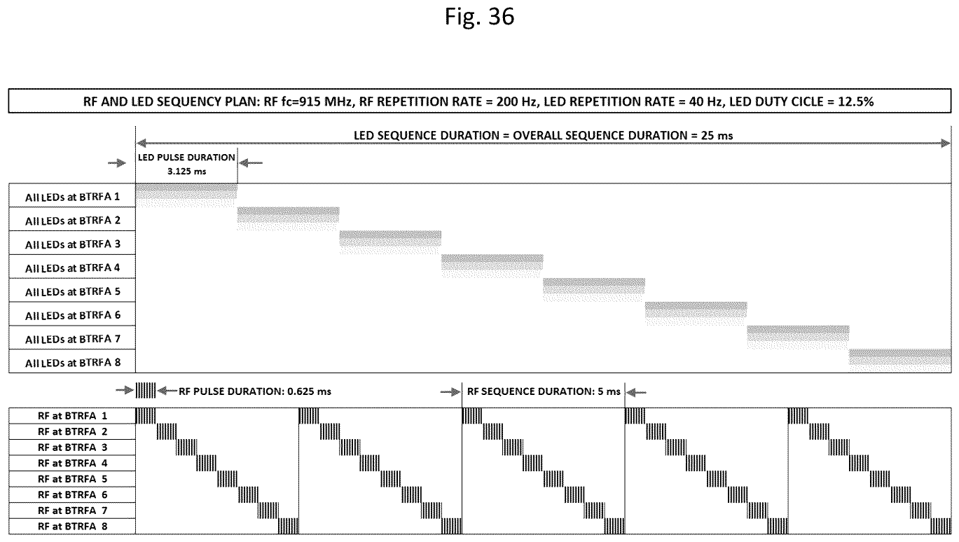

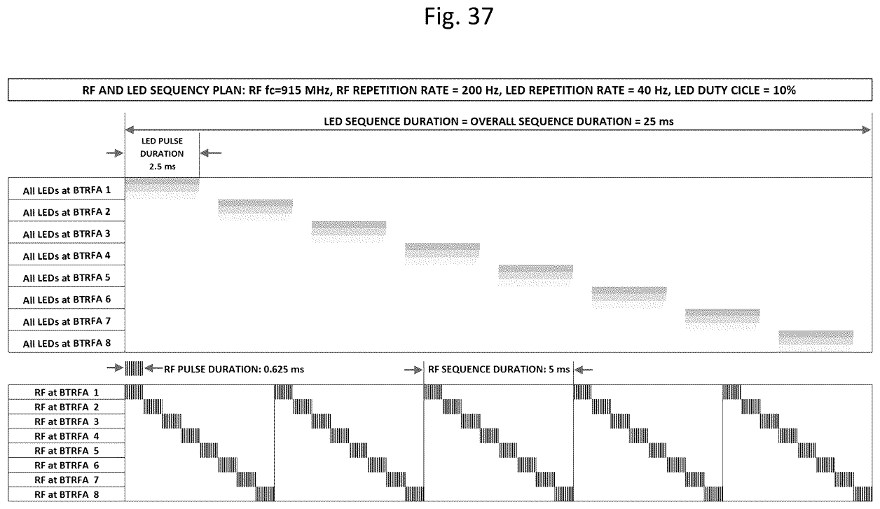

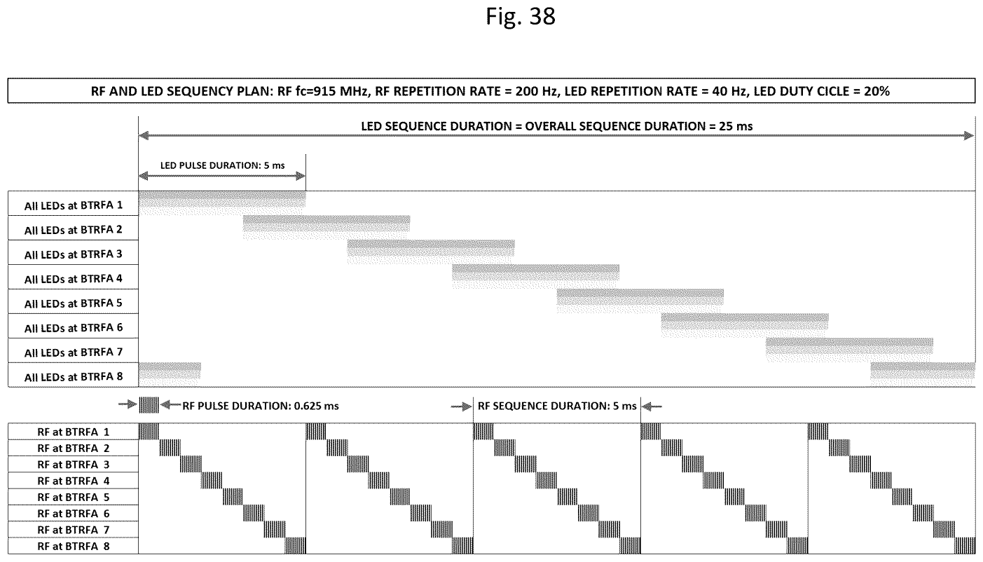

The electromagnetic field emitted by the BTRFA (50) may be synchronized with the red and NIR light signals from the LEDs (200) by adjusting their duty cycle within the combination of the BTRFA-LEDs. Possible synchronization configurations include BTRFA (50) emitting at 915 MHz with a 200 Hz repetition frequency and 100% duty cycle, while LEDs (200) emit red/NIR signals at 40 Hz with a duty cycle of 12.5% (FIG. 36), 10% (FIG. 37), or 20% (FIG. 38).

According to a second aspect, the present invention relates to the use of BTRFA (50) as described above for direct administration of an electromagnetic field to a targeted human tissue, and a method of directly administering an electromagnetic field to a targeted human tissue, comprising positioning BTRFA adjacent or in direct contact to the human tissue, thereby allowing direct administration of said electromagnetic field or signals or waves. Advantageously, the present invention also relates to the use of the BTRFA (50) in combination with LEDs (200) as described for direct administration of the synergistic combination of pulsed electromagnetic signals and with red/NIR light to body tissue in subjects in need of such treatment, and relates to a method of directly administering such synergistic combination to a targeted human tissue, comprising positioning the BTRFA-LED combinations adjacent or in direct contact to the human tissue, thereby allowing direct administration of said electromagnetic field signals with the red/NIR signals.

Targeted human tissues which may be treated by or exposed to such electromagnetic signal may include any parts of the human body, such as for example and without any limitations, the human head or skull, the frontal part and/or the temporal parts, and/or the occipital part of the head, the neck, the abdomen of a human body. Preferred human body tissues comprise the human head, optionally the neck and possibly abdomen. Most preferred human body tissues comprise the human head.

Such BTRFA (50) and/or BTRFA-LED combinations are particularly useful for use in a method of for treating, preventing, stabilizing and/or reversing the symptoms of neurologic disorders, such as neurodegenerative diseases such as Alzheimer's disease, Frontotemporal dementia and variants thereof, cerebral amyloid angiopathy, Parkinson's disease, Lewy body dementia, in a subject in need thereof, comprising placing the BTRFA (50) at proximity to the human head of a subject in need thereof and allowing direct transcranial administration of said electromagnetic waves or field to the head of said subject.

The BTRFA (50) as described above may be used for preventing and/or alleviating brain injuries, such as concussions, traumatic brain injuries, and/or post stroke disorders, as well as for treating, preventing and/or mitigating depression, migraine headaches, myodesopsia, and/or tinnitus in a subject in the need therefor, comprising placing the body tissue radiofrequency applicators (50) at proximity to the human head of a subject in need thereof and allowing direct transcranial administration of said electromagnetic waves or field to the head of said subject.

The BTRFA (50) as described above may further be used by healthy patients for general wellness purposes, particularly for relieving headaches and signs of fatigue, and/or enhancing general mental and cognitive abilities.

The whole head and brain, or specific parts of the head, such as the frontal lobe, and/or the parietal lobe, and/or the occipital lobe, and/or the temporal lobe may be thus transcranially exposed to pulsed electromagnetic field or pulsed RF signal and advantageously exposed to pulsed red and NIR signals as described above.

As described herein above, BTRFA (50) and BTRFA-LED combination according to the present invention generate pulsed electromagnetic waves and red/NIR signals which efficiently penetrate within the human skull and human cortex. In effect, 80-90% of the emitted power of the pulsed RF signal and 20% of the red/NIR signals are absorbed by the head of the subject. BTRFA thus provides a reliable, reproducible, and efficient specific absorption rate (SAR) within human brain tissue. BTRFAs and BTRFA-LED combinations according to the present invention are thus particularly useful for transcranial administration of said electromagnetic waves alone or in combination with the red and NIR signals toward the skull and brain of a subject and thus may be advantageously embedded into a medical head device.

As explained above, the BTRFAs (50) according to the present invention may have variable shapes and sizes to fit and adapt to various subject skull shapes and sizes, thereby enhancing coverage of whole brain or of specific lobes of the brain of the subject. In addition, they may advantageously be designed with inwardly curved/bent shapes in both length and width dimensions and thus be positioned at proximity of rounded portions of the skull, such as for example the frontal, parietal, temporal, occipital parts, to further optimize enhanced coverage to whole brain regions and/or specific cortical brain regions. Furthermore, as described above, X-BTRFA (130) and/or Y-BTRFA (120), and Z-BTRFA (140) may be selected according to the desired zone and shape of electromagnetic signal and desired power and SAR distribution within the desired area of the treated human tissue.

Another important advantage of the body tissue radiofrequency applicators (50) of the present invention is that the resonance frequency is significantly widened, thereby allowing good matching when exposed at different distances to the head of a human subject.

According to a third aspect, the present invention thus also provides a novel non-invasive brain stimulation head device comprising one or more BTRFAs (50) alone or in combination with one or more LEDs (200) as described above.

Said one or more BTRFAs (50) may thus be embedded within the medical head device proximal to the head of a subject when the device is worn by the subject, so that the electromagnetic signal is inwardly directed towards the head of the subject. In addition, the body tissue radiofrequency applicators are spaced apart to apply a homogenous pulsed electromagnetic energy or field directed toward the head of the subject without no or minimal overlap of the pulsed electromagnetic energy or field. The head and cortex of the subject thus may receive directional and homogeneous exposure of electromagnetic field. This allows for a more focused treatment, without as much power loss from radiation going into the air away from the head.

The head device according to the present invention may thus also comprise a head unit, such as for example a head cap, a helmet, or a headset which holds the array of said one or more body tissue radiofrequency applicators at proximity and in predetermined positions relative to the head of a subject. Said one or more BTRFAs (50) are held by and within the head unit and connected by flexible means to each other, to adapt to different head sizes of subjects. Each of them may be enclosed in separate cases which are connected to each other by flexible connecting means, thereby allowing the BTRFA units to be pressed against the subject head, hair, and skull.

The head device may thus comprise BTRFAs (50) as described above and which may be chosen among X-BTRFA (130), Y-BTRFA (120), and/or Z-BTRFA (140), and having any of the configurations as described above, depending on the targeted body tissue area and desired depth of penetration, and in order to take advantage of their specific distinct characteristics. As described herein above, X-BTRFA (130) provides a uniform distribution over a broader tissue volume with a wider area of exposure with very low radiation losses. Y-BTRFA (120) provides a greater depth of penetration of electromagnetic waves into living tissue, while maintaining an unchanged coverage width and slightly higher levels of SAR at the center. Z-BTRFA (140) provide a maximum depth of penetration with the highest SAR value in significantly smaller volume and surface area of tissue.

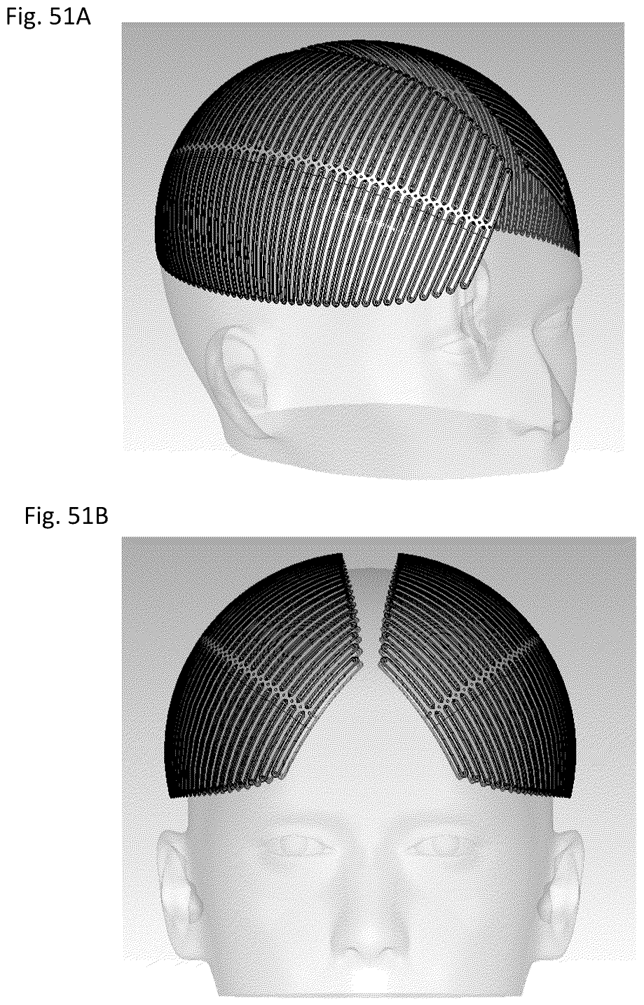

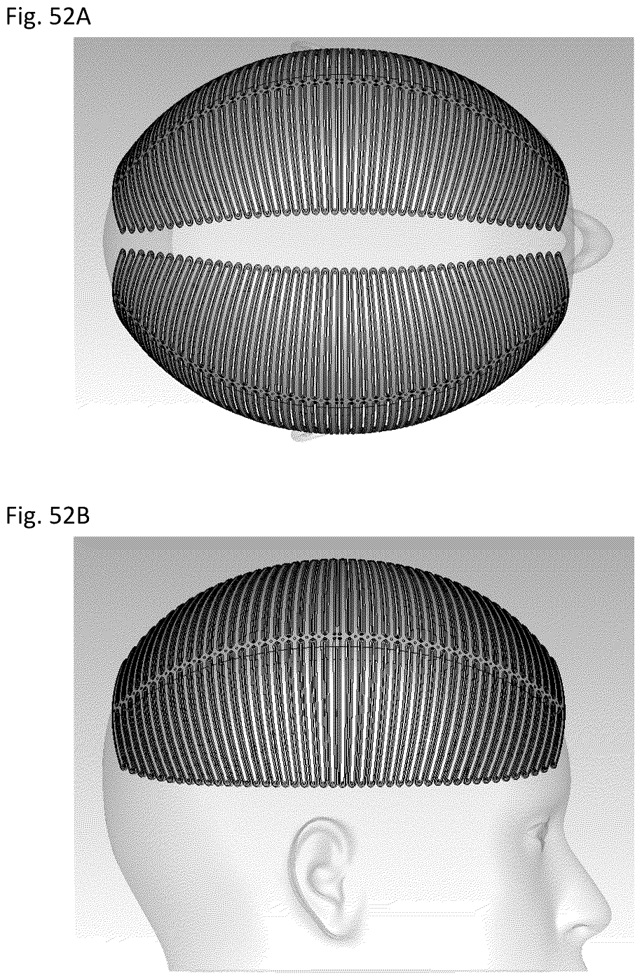

According to one embodiment, the non-invasive brain stimulation head device according to the present invention may comprise BTRFAs emitting a pulsed electromagnetic field or signal at a frequency in the VHF range, namely ranging from 30-300 MHz, 30-200 MHz, 40-250 MHz, 50-200 MHz, 50-150 MHz, 100-150 MHz, 60-100 MHz, 30-70 MHz, 50-65 MHz, 60-65 MHz. Said non-invasive brain stimulation head device then preferably comprises one or two large BTRFA (50) highly compact and meandered BTRFA (50) covering the whole skull or head of the subject as shown in FIGS. 51 and 52.

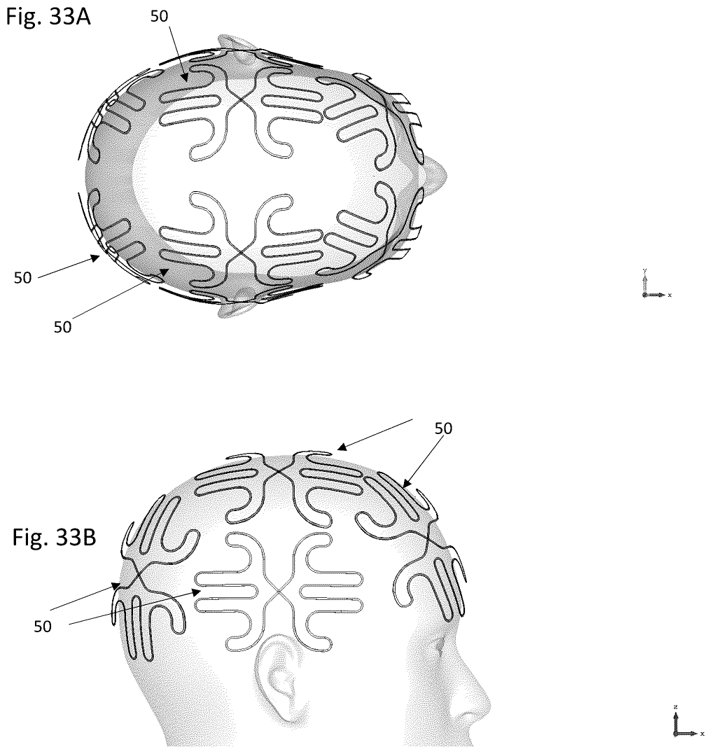

According to another embodiment, the non-invasive brain stimulation head device according to the present invention may comprise BTRFAs emitting a pulsed electromagnetic field or signal at a frequency in the UHF range, namely ranging from 300-3000 MHz, 400-1500 MHz, 500-1300 MHz, 800-1100 MHz, 850-950 MHz, or 800-930 MHz, or 850-915 MHz, 910-920 MHz. Said non-invasive brain stimulation head device then preferably comprises 1-16, or 6-10, or 8-10 BTRFAs (50) units having a bow-tie complex butterfly shape. An example of device comprising eight bow-tie complex butterfly-shaped BTRFAs is illustrated in FIG. 33.

According to both embodiments, the non-invasive brain stimulation head device is configured such that the energy specific absorption rate (SAR) within a treated or targeted human tissue, such as human cortex, is in a range of from 0.2 to 10 W/kg or from 0.2 to 3 W/kg, 0.5 to 2.5 W/kg, or 1 to 2 W/kg.

The non-invasive brain stimulation head device according to the present invention may thus comprise an array of said one or more BTRFAs which may be activated sequentially or in combination to produce a radiation pattern that is used for the treatment of the brain. Preferably, the BTRFA may be radiating on and off in sequential order. Therefore, the device may be a multi-emitter head device, wherein when one BTRFA is off, another BTRFA may be on, so that each BTRFA emits in a sequential fashion, one after the other. This also allows for a single therapy waveform generator to be shared by multiple applicators.

The subject wearing the non-invasive brain stimulation head device may receive a homogeneous exposure of a pulsed electromagnetic signal and does not experience any uncomfortable sensation of heat or pain. Indeed, said above-described pulsed RF electromagnetic energy does not raise the temperature of the targeted body tissue.

The non-invasive brain stimulation head device may have a single BTRFA which is active at a time, or one or more BTRFA which may be activated in sequence or simultaneously. Preferably, said one or more BTRFA of the head device are delivering pulsed electromagnetic waves in a sequential manner such that no two applicators are simultaneously delivering or discharging.

Alternatively, said one or more BTRFAs may emit a different frequency. A first BTRFA may be radiating at a high frequency while a second BTRFA radiates at a lower frequency, and the radiating characteristics may switch such that the first BTRFA is radiating at a low frequency while the second BTRFA radiates at the high frequency. A specific frequency could be distributed to multiple emitters at the same time, or frequencies may be generated and distributed to the body tissue radiofrequency applicators. For example, the head device may thus comprise one or more BTRFA (50) and three different RF frequencies to ensure total penetration and coverage into each of the predetermined locations.

Advantageously, said one or more BTRFAs (50) as described above may be used alone or may be combined, within the wellness or medical head device according to the present invention, with an array of one or more LEDs (200) as described above configured for emitting red and/or near-infrared signals, thereby allowing administering a synergistic combination of one or more pulsed electromagnetic signals either alone or in synergistic combination with one or more pulsed RED and near-infrared (NIR) light to the body tissue of a subject in need thereof.

The combinations of said one or more BTRFAs (50) and the array of one or more LEDs (200) may be embedded within or attached to said head device, wherein said medical head device is configured to fit on head of a subject, and wherein said combined one or more BTRFAs (50) and of one or more LEDs (200) are positioned such that they are adjacent to said head when the medical head device is worn by the subject. For example, BTRFA-LED combinations may be enclosed in hollow cases which are connected by flexible or elastic connection means are embedded within the medical head device together with the whole electronic circuit (mother boards) allowing the functioning thereof.

The head device according to the present invention may be positioned over the head of a subject, thereby allowing said one or more BTRFAs (50) either alone or in combination with the LEDs (200) as to deliver to the whole head and brain, or specific parts of the head and brain, such as the frontal lobe, and/or the parietal lobe, and/or the occipital lobe, and/or the temporal lobe, a therapeutically effective amount of the electromagnetic field or signal optionally with a therapeutically effective among of red and NIR signals.

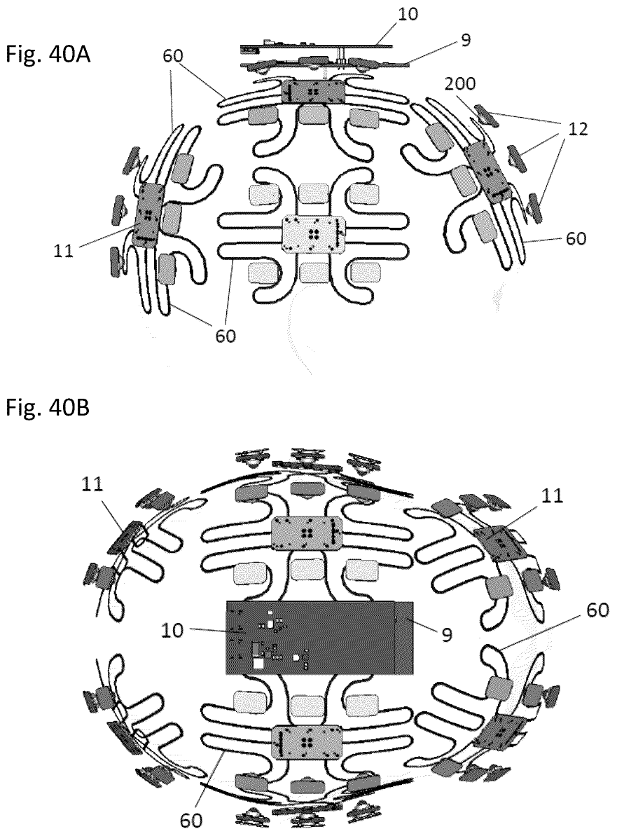

The head device may comprise one or more BTRFAs (50) and may further comprise a total number of LEDs (200) ranging from 10 to 100 or 20 to 90, or 30 to 70 or 40 to 60 LEDs. FIG. 40 illustrates a head device comprising eight BTRFAs (50) carrying six LEDs (200) per each BTRFA.

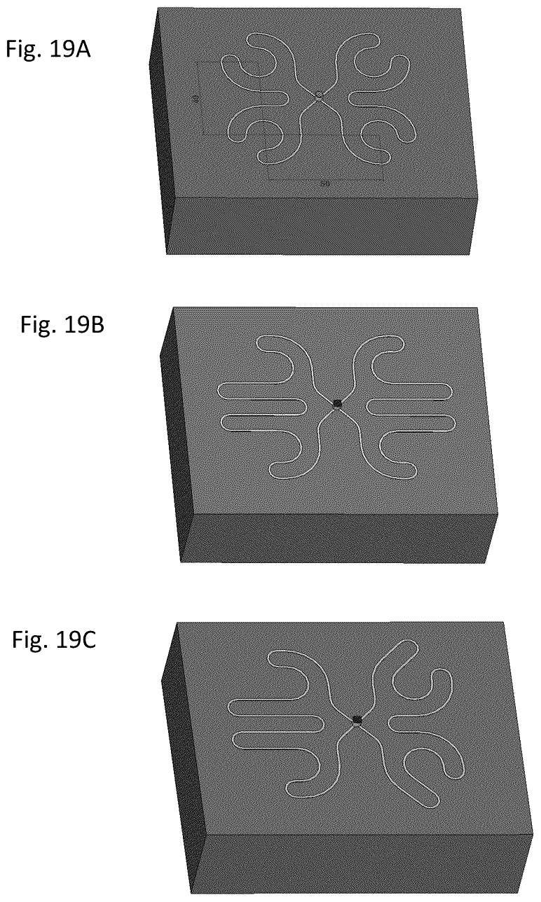

Within the wellness or medical head device, the BTRFA units (50) and LEDs (200) are spatially arranged to ensure a preferably homogeneous application of pulsed electromagnetic energy and red/NIR light to the head, with minimal or no signal overlap. Depending on the number of LEDs per BTRFA, the LEDs (200) may be spaced 30-60 mm apart, preferably 40-50 mm. The BTRFAs (50) may be slightly modified in shape to accommodate the LEDs (200) while ensuring unimpeded RED and NIR light emission. FIGS. 19A-C illustrate exemplary modifications, including rounded extremities of the BTRFA.

The BTRFAs (50) are preferably positioned at 1-4 mm from the skull, more preferably 2-3 mm, while the LEDs (200) are placed at 8-14 mm, more preferably 10-14 mm, and ideally around 12 mm from the skull.

The electronic control unit/system comprises electronic means to generate the therapeutic pulsed electromagnetic signal and means for signal amplification. For example, the electronic control unit/system may comprise (a) a single transmitter to sequentially drive antenna sets, (b) a switching device to select for each activation period in an activation sequence of said one or more BTRFA to be driven, and a controller. The controller, for each activation sequence determines a power output of each BTRFA and generates an adjusted control signal for the single transmitter such that the power output of at least one BTRFA is the same as an average value, regardless of the load of the BTRFA.

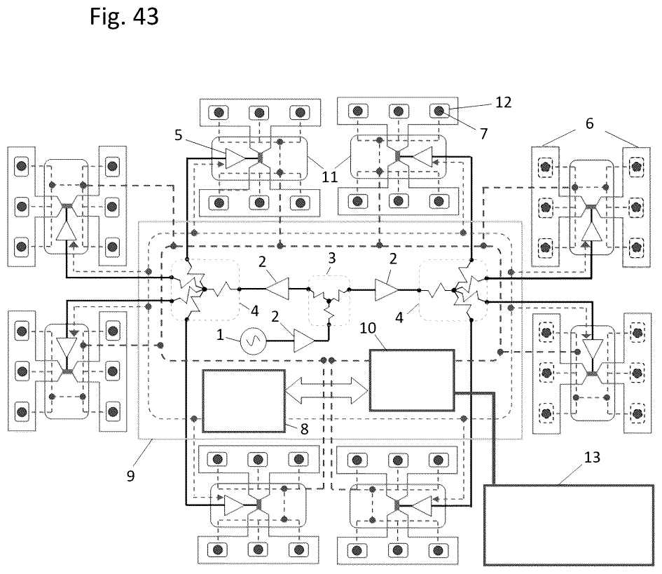

An example of the electronic circuit is depicted in FIG. 43. Such electronic circuit comprises:
(i) a RF central generator (1) which may be a voltage-controlled oscillator, able to generate the required frequency of the pulsed electromagnetic signal as described above;
(ii) an appropriate number of amplifiers with sufficient gain and output power capabilities as required by the next amplifying stage and for compensation of the power losses introduced by resistive power dividers (3) and (4) or, in the case of output amplifier (5) to achieve the sufficient output level of RF signal at the BTRFA input in order to achieve the required SAR value within a treated body tissue, such as for example three pre-amplifiers: a first amplifier (2) allowing the achieve the power which is connected via short printed microstrip RF transmission line and a resistive power divider 1 to 2 (3) to two second stage amplifiers (14);
(iii) the second stage amplifiers (14) being connected to the RF central generator (1) via resistive RF power divider 1 to 2 (3), and connected to the PCB for output amplifier (11) via resistive RF power divider 1 to 4 (4);
(iv) eight PCBs (11) each carrying one output amplifier (5) and one BTRFA (6) and connected to the central PCB unit (9) by flexible coaxial cables carrying RF signal from outputs of resistive RF power divider 1 to 4 (4). Each of eight PCB units (11) is also connected by flexible wires to the power supply unit (10) as well as to the control unit (8) that provides pulsed digital signals for switching on and off the output amplifiers (5). Each of the eight PCB units (11) are connected by flexible conductive wires, providing pulsed power supply, with six 3 in 1 LED diodes (7) each placed on small separate PCB (12) that provides cooling and mechanical support for the LEDs (7).
(v) a means for generating synchronizing digital signals at the repetition rate of 200 Hz (or similar) for switching on and off the output amplifiers (5) in accordance to the sequence plan, which may be a microprocessor or with other digital components (flip flop type); and
(vi) a means for generating synchronizing digital signals at the repetition rate of 40 Hz (or similar) for switching on and off the 3 in 1 LEDs (7) in accordance to the sequence plan, which may be a microprocessor (the same used for (v) or different) or with other digital components (flip flop type).

The medical head device according to the present invention may have a control box for ON/OFF switching by the patient or the caregiver, a battery, and optionally a timer, and/or means for shielding out-radiation, and/or feedback means.

For practical and convenient use of the non-invasive brain stimulation head device, means for generating and amplifying the therapeutic pulsed electromagnetic waves as well as RED & NIR lights is as small as possible, allowing a patient to wear the device while being able to move around in his home, either reading, watching TV, sleeping, cooking, etc.

The control box for activating said one or more BTRFA and LED units may be either part of the head device or separated from the head device and connected to it via a cable. Preferably, the wellness head device or medical head device comprises a head unit with BTRFA and LED units connected to the controller or control box which can be worn on the arm or conveniently placed nearby for example on a table, a chair back, etc.

The power supply may be any suitable battery, such as a rechargeable battery or energy-harvesting system integrated into the head device.

The timer may be for example a stop-timer which automatically turn the device off at the end of the use of the subject. The medical device may also include a treatment status indicator which may indicate that treatment is complete, for example via an audio, visual or tactile indicator.

Means for shielding out-radiation allow eliminating EM waves radiating away from the subject head when the head unit is positioned over the head of the subject. Such means may comprise shield.

Feedback means may be used to adjust treatment parameters. For example, if a particular patient is responding better to a particular frequency within the therapy, the treatment may be adjusted to use this favorable frequency more.

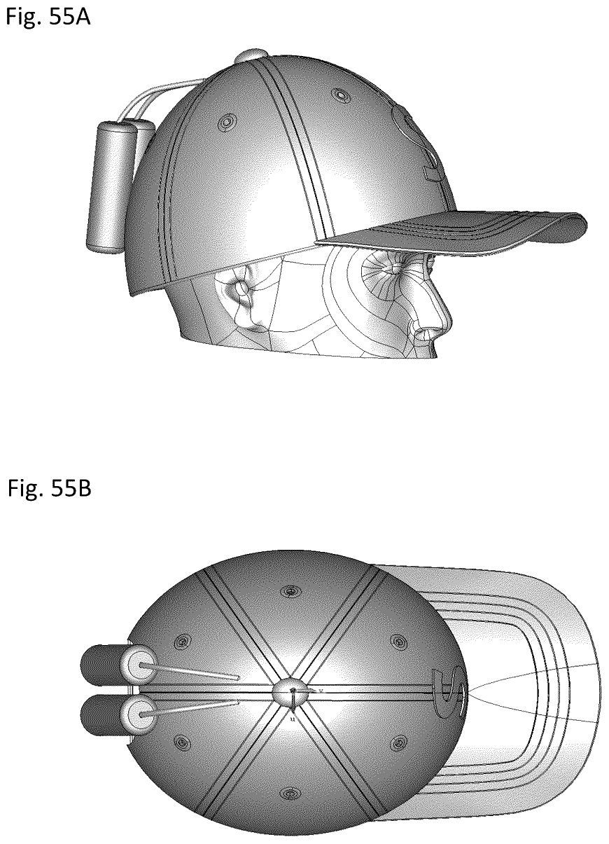

Said non-invasive brain stimulation head device described above may be a wellness head device for wellness use or a medical head device for medical use. It may be present in the form of an intelligent wearable hat or augmented hat as shown in FIG. 55.

Said intelligent wearable headgear may comprise a cap with semi-rigid structure which may conform with the user's head, optionally with an integrated grid structure, which may further support multiple electronic components for monitoring, stimulating, or processing neurological signals. The invention is particularly useful in cognitive enhancement applications.

In addition to the BTRFAs (50) and/or LEDs, the intelligent head device may comprise multiple sensors, such as neurological sensors, processing units, and wireless communication modules. The cap fits comfortably on the head of the subject while maintaining precise sensor positioning.

Neurological sensors may comprise, for example electrodes capable of detecting brain activity, in particular electroencephalogram (EEG) electrodes, near-infrared spectroscopy sensors, or magnetometers.

Processing units may comprise embedded microcontrollers or AI-driven chips for data analysis. These may include a machine learning algorithm for analyzing brainwave patterns and providing real-time feedback.

Wireless communication modules enable real-time data transmission to external devices for real-time analysis. They may include Bluetooth, Wi-Fi, or 5G connectivity.

According to a fourth aspect of the present invention, BTRFA and non-invasive medical head device as described above may be used in a method of treating and/or preventing neurodegenerative proteinopathies and/or diseases, as well as a method of stabilizing and/or reversing symptoms of neurodegenerative diseases, such as Alzheimer's disease (AD), mild cognitive Impairment (MCI) and amnestic mild cognitive impairment (aMCI), Parkinson's disease, cerebral amyloid angiopathy, dementia with Lewy bodies (DLB) including dementia with Lewy bodies (DLB) and Parkinson's disease dementia (PDD), frontotemporal dementia, variants thereof such as semantic dementia, primary progressive aphasia, and Pick's disease, in a subject in need thereof, comprising administering the combination of said pulsed electromagnetic signal generated by the one or more body tissue radiofrequency applicators with said red and/or near-infrared signals generated by the one or more LEDs. Treatment and/or prevention of these neurodegenerative diseases comprise alleviation of both brain pathologies and behavioral symptoms.

The present invention thus provides a method of treating and/or preventing neurodegenerative proteinopathies and/or diseases, as well as a method of stabilizing and/or reversing symptoms of neurodegenerative diseases, such as Alzheimer's disease (AD), mild cognitive Impairment (MCI) and amnestic mild cognitive impairment (aMCI), Parkinson's disease, cerebral amyloid angiopathy, dementia with Lewy bodies (DLB) including dementia with Lewy bodies (DLB) and Parkinson's disease dementia (PDD), frontotemporal dementia, variants thereof such as semantic dementia, primary progressive aphasia, and Pick's disease, in a subject in need thereof, comprising positioning the BTRFA or medical head device, at close proximity to the head of the subject, activating said one or more BTRFA in combination and simultaneously with the array of said one or more red and NIR lights, and transcranially exposing the head and cortex of the subject to a therapeutically efficient dose of pulsed electromagnetic waves or field at a specific frequency and a specific absorption rate (SAR) and to red and NIR lights or LEDs at specific wavelengths as described above.

By activating the array of said one or more BTRFA in combination and simultaneously with the array of said one or more red and NIR lights, the head and cortex of the subject may transcranially receive a therapeutically efficient dose of pulsed electromagnetic waves or field at a specific frequency and a specific absorption rate (SAR) and to red and NIR lights as described above for a predetermined absorption period.

The term dementia was first used in the early 19th century. At that time, dementia was regarded as a form of what was called “mental alienation”, which also included schizophrenia and mood swings. Today, various types of dementia are distinguished, but all have a common definition that, on the basis of World Health Organization diagnostic criteria, may be stated as follows: “a gradual loss of memory and of the ability to form and organize ideas, severe enough to interfere with activities of daily living, and present for at least six months”. The cognitive and social problems associated with dementia result not from psychiatric disorders, but rather from organic causes that have been well characterized: specifically, an abnormally high number of neurons deteriorating and dying in certain parts of the brain. In this sense, the various forms of dementia are part of the broader category of neurodegenerative diseases.

One of the most common neurodegenerative diseases or dementia is Alzheimer's-type dementia, commonly known as Alzheimer's disease. Alzheimer's disease (AD) was first described and diagnosed by Dr. Alois Alzheimer in 1906. AD is characterized as a severe, chronic, and progressive neurodegenerative disorder. With life expectancy continually growing increasing, the number of AD cases is growing drastically and treating AD patients is becoming urgent. According to WHO. AD is the most common cause of dementia, accounting for as many as 60 to 70% of senile dementia cases and affecting 55 million people worldwide.

In Alzheimer's disease pathogenesis, the neuropathological hallmarks are the buildup of extracellular amyloid plaques (amyloidosis) and neurofibrillary tangles (tauopathies). The major components of amyloid plaques are Aβ₁₋₄₀ and Aβ₁₋₄₂ peptides, which aggregate and form β-sheets. The Aβ peptides are produced by the proteolytic cleavage of the amyloid precursor protein by the beta-amyloid cleavage enzyme alpha, and beta and gamma secretases. The Aβ₄₀ is the predominant isoform of amyloid fragments detected in plasma and cerebrospinal fluid samples, while the Aβ₄₂ isoform was mainly associated with nucleation, due to its aggregation tendency. In parallel to the extracellular amyloid plaque deposition in AD brains, the Aβ intracellular deposition triggers the pathological cascade involving a second neurotoxic molecule: the tau protein which is normally involved in the formation and stabilization of microtubules. Therefore, AD is also characterized by the deposition of the intra-neuronal neurofibrillary tangle (NFT) of hyper-phosphorylated aggregated tau protein. Several lines of evidence suggest that the small oligomeric forms of amyloid-β and tau act synergistically to promote synaptic dysfunction in Alzheimer's disease (AD). These aggregates and plaques are very toxic for the neurons and the pathology gradually result in the loss of neuronal connections, death of the neurons (mostly pyramidal neurons of the temporal cortex) and the destruction of the nervous system.

Beta-amyloid plaques and tau aggregates are initially deposited primarily in the hippocampus, the entorhinal cortex posterior and the cingulate cortex which are critical areas for memory and spatial orientation. Over time, the pathology becomes more widespread, affecting the entire cerebral cortex. The signal is disrupted among the neurons which completely stop working and lose connection with other neurons, thereby leading to brain atrophy (loss of neurons), memory loss, confusion, mood swings, personality changes, and difficulty in performing even basic routine tasks or daily functions.

International standards for the diagnosis of Alzheimer's disease have been established based on medical history, neuropsychological testing, clinical examination, and laboratory assessments. Criteria for the clinical diagnosis of Alzheimer's disease have first been defined and published in 2011 by the NIA-AA working group. These criteria, which are commonly referred to as NIA-AA criteria, have been reliable for the diagnosis of Alzheimer's disease (Lopez O L et al. Cuff Opin Neurol. 2011 December; 24(6): 532-541. doi:10.1097/WCO.0b013e32834cd45b) as they take into consideration different biomarkers to support the clinical diagnosis of the different stages of the disease. These criteria, which are commonly referred to as the NINCDS-ADRDA criteria are regularly revised. The most recent revision was published in 2011 (McKhann G et al., Alzheimers Dement. 2011 May; 7(3): 263-269. doi:10.1016/j.jalz.2011.03.005). According to the WHO Alzheimer's disease typically progresses slowly in three general stages: mild, moderate, and severe. (https://www.who.int/news-room/fact-sheets/detail/dementia).

The progression of Alzheimer's disease can be broadly divided into seven stages. During the first two stages, often referred to as the mild stage, it is challenging to determine whether a person is suffering from the disease. Alzheimer's typically acts as a silent condition, with no obvious symptoms appearing in the early stages unless the patient undergoes clinical diagnosis. As the disease progresses, patients begin to show mild changes in their behavior. The third and fourth stages, known as the moderate stages, are usually the longest and can last for several years. During these stages, the disease starts to escalate. Although the person may still function independently, memory lapses become more frequent and severe, leading to increasing difficulties in daily activities and language use. As Alzheimer's continues to progress, the individual requires more extensive care and support from family members and caregivers. From the fifth stage onward, classified as the severe stage, patients may begin to lose recognition of important aspects of their identity and experience additional neurological challenges, such as difficulties with speech and movement. Mood swings, distrust in others, irritability, agitation, and delusions become more common, significantly impacting the patient's overall well-being.

BTRFA, non-invasive medical head devices, and methods according to the present invention are particularly useful for preventing and/or stabilizing and/or reversing the symptoms of Alzheimer's disease (AD) of a subject at any stage of the AD, e.g., either mild, moderate and/or severe stages according to National Institute of Neurological and Communicative Disorders and Stroke-Alzheimer's Disease and Related Disorders Association (NINCDS-ADRDA) criteria. They may be administered or applied to the head of said subject for one treatment each day or for multiple, spaced-apart treatments each day. Regime of administration may consist for example applying or positioning the medical head device of the invention onto the head of a subject for 15-60 min or for 30-60 min, preferably either once a day or twice a day, so that therapeutically effective doses of pulsed electromagnetic signal generated by the one or more body tissue radiofrequency applicators (50) and of pulsed red and/or near-infrared signal generated by the one or more LEDs (200) is simultaneously administered or applied to the head of said subject.

Combination of these signals provide a deeper penetration through the meninges, cranial material, and then through the brain matter. In addition, simultaneous stimulations with these signals are expected to provide synergistic molecular and biological effects which are manyfold. These synergistic effects include an increase of mitochondrial activity with an increase of adenosine triphosphate (ATP) production and thus an enhanced metabolic capacity, thereby contributing to overcome the low ATP level associated with many neurological disorders, as well as increased anti-inflammatory effect, possibly via the inhibition of the cyclo-oxygenase 2 (COX-2 enzyme), the inhibition of NF-kB and tumor necrosis factor (TNFα), as well as a decrease of the oxidative stress and the decrease of reactive oxygen species (ROS) production. In addition, we expect a beneficial regulation of other pro- and anti-inflammatory cytokines in brain parenchyma wherein pro-inflammatory macrophages with M1 phenotype present in the brain of subjects are triggered to switch to M2 anti-inflammatory phenotype enabling the phagocytosis and degradation of the amyloid plaques and beta amyloid aggregates by newly active M2 macrophages and microglia. Synergistic effects further include increased hemoglobin oxygenation and increased vasodilation, which leads to improved cerebral blood flow, oxygenation, and nutritional supplementation to the brain parenchyma.

There is a battery of neuropsychological tests to identify symptoms and to monitor the efficacy of the treatment on the progression of Alzheimer's disease. Most widely used neuropsychological tests include ADAS-Cog-Plus or ADAS-Cog14 (Alzheimer's Disease Assessment Scale-Cognitive Subscale 14) and MMSE (Mini Mental State Examination), logical Memory Tests I and II by around 3.0 points, Trail Making Tests A and B, Boston Naming Test, Auditory Verbal Learning Tests before and after at least 2-month treatment.

Alzheimer subjects at mild, moderate and even severe stages, having used the non-invasive medical head device according to the present invention for 1 hour twice daily for at least two consecutive months with at least 7-hour interval between those daily uses, are expected to experience positive cognitive and executive changes, improvements of the quality of life with improved sleep, less anxiety and agitation, less apathy and/or improved mood and energy as well as decreased burden of the caregivers.

The subject may see improved cognitive performance of Alzheimer's disease patients and reverse cognitive decline as measured by neuropsychological tests, or in any tests described in the Examples below, such as for example ADAS-cog14, MMSE, or Rey AVLT (Rey Auditory Verbal Learning Test).

In addition, synergistic effects are expected in terms of increased degradation and clearance of beta-amyloid plaques and of amyloid beta oligomers in the plasma and the cerebrospinal fluid of the subject. More precisely, increased destabilization of insoluble amyloid-β plaques and/or neurofibrillary tangles within the brains of AD patients; and/or dissociation of insoluble amyloid-β deposits in senile plaques into soluble Aβ oligomers/monomers and/or of the neurofibrillary tangles; and/or elimination of the soluble Aβ oligomers/monomers and tau monomers in the cerebrospinal fluid and plasma of AD patients.

AD subjects are expected to have a change from baseline of Alzheimer's markers such as beta-amyloid peptides 1-40 and 1-42, total tau (t-tau), and phospho-tau (p-tau) in blood and CSF prior and upon completing 2-month treatment with device described above. In particular, increased levels of Aβ₄₀ and Aβ₁₋₄₂ soluble monomeric peptides and of oligomeric Aβ aggregates in the plasma and in cerebrospinal fluid (CSF) are expected following to a 2-month treatment with medical head device according to the present invention. Plasma and CSF levels of beta-amyloid peptides 1-40 and 1-42, total tau (t-tau), and phospho-tau (p-tau) may be easily analyzed using ELISA tests.

In addition, subjects early, mild or moderate Alzheimer or their caregivers may notice significant improvements in their sleep, less anxiety and agitation, less apathy and/or improved mood and energy.

Mild Cognitive Impairment (MCI) is a condition characterized by cognitive decline that is greater than expected for a person's age and education level but does not interfere significantly with daily life activities. Amnestic Mild Cognitive Impairment (aMCI) is a subtype of MCI where memory loss is the predominant cognitive impairment. MCI is characterized by cognitive decline that is greater than expected for a person's age and education level with diverse causes ranging from vascular issues to early-stage neurodegenerative diseases. Indeed, etiologic causes include vascular factors such small vessel disease, stroke, or other cardiovascular conditions, but neurodegenerative diseases such as early-stage Alzheimer's disease, Parkinson's disease, and other neurodegenerative conditions can manifest as MCI. It may be caused by Metabolic and systemic conditions, and even by psychiatric conditions, such as depression and anxiety. The symptoms of MCI include general cognitive decline, including issues with memory, attention, language, and executive function. MCI is typically considered an intermediate stage between normal cognitive aging and dementia. It can progress to more severe cognitive decline or remain stable. Several tests and biomarkers may be used to detect and monitor the progression of the disease. Tests may include for example Montreal Cognitive Assessment (MoCA), Mini-Mental State Examination (MMSE), and Clock Drawing Test. Biomarkers vary depending on the underlying cause but may include neuroimaging (MRI for structural changes, PET scans for metabolic activity), and cerebrospinal fluid (CSF) analysis for protein levels (e.g., amyloid-beta, tau).

aMCI is primarily associated with early-stage Alzheimer's disease, where the amyloid plaques and neurofibrillary tangles begin to affect the hippocampus and related brain regions responsible for memory. aMCI is often viewed as a precursor to Alzheimer's disease, with a higher likelihood of progressing to dementia. It involves the same pathological processes as Alzheimer's disease, with early amyloid deposition and tau-related neurofibrillary tangles beginning in the hippocampus and entorhinal cortex. Predominant symptoms are memory impairment, especially difficulty recalling recent events, names, or appointments. Other cognitive domains are less affected, and the ability to perform daily tasks is mostly intact. The Petersen criteria are often used, requiring memory complaints (preferably corroborated by an informant), objective memory impairment, largely preserved general cognitive function, and intact daily activities, with no dementia. Several tests and biomarkers may be used to detect and monitor the progression of the disease. Such cognitive tests may be specific tests for memory, such as the Rey Auditory Verbal Learning Test (RAVLT) and the California Verbal Learning Test (CVLT), which assess delayed recall and recognition memory. Biomarkers indicative of Alzheimer's disease are also used to monitor aMCI, such as decreased amyloid-beta 42 and increased total tau or phosphorylated tau in CSF, as well as amyloid PET imaging showing amyloid plaques in the brain.

Parkinson's disease (PD) is the second most common neurodegenerative disorder after Alzheimer's disease. It primarily affects movement control. It is characterized by the loss of dopamine-producing neurons in the substantia nigra, a region of the brain that plays a critical role in regulating movement. PD is the second most common neurodegenerative disorder after Alzheimer's disease. PD is caused by a combination of genetic and environmental factors, with oxidative stress and mitochondrial dysfunction playing a role in its pathogenesis. The core pathophysiological feature of PD is the progressive loss of dopaminergic neurons in the substantia nigra pars compacta, leading to dopamine depletion in the striatum, a key region involved in motor control. Such neuron loss is caused by intracellular inclusions composed of alpha-synuclein and other proteins are found in the brains of PD patients. These Lewy bodies are also associated with neuronal death. The loss of dopamine disrupts the normal functioning of the basal ganglia, leading to the characteristic motor symptoms of PD, including tremors, rigidity, and bradykinesia. There are characteristic motor and non-motor symptoms.

Motor symptoms comprise resting tremors, bradykinesia, e.g., slowness of movement and difficulty initiating movements, rigidity, postural instability, cognitive impairment, mood disorders such as depression, anxiety, and apathy, as well as sleep disturbances.

PD progresses from mild, unilateral symptoms to severe disability requiring full-time care. Stage 1 or mild wherein symptoms typically affect only one side of the body (unilateral) and daily activities are generally not impaired. Stage 2 or moderate wherein symptoms become bilateral, but there is no impairment of balance, and daily tasks may take longer but can still be completed. Stage 3 or mid-stage wherein balance becomes impaired, and falls become more common. The individual is still fully independent but experiences significant difficulty with tasks. Stage 4 or advanced wherein severe disability, requiring help with daily activities, and wherein the person can still walk or stand unassisted but with great difficulty. Stage 5 or severe wherein the individual is often bedridden or confined to a wheelchair, and full-time care is required.

The diagnosis of Parkinson's Disease is primarily clinical, based on the criteria established by the Movement Disorder Society (MDS) and the UK Parkinson's Disease Society Brain Bank. Key diagnostic criteria include bradykinesia plus at least one of the following: resting tremor, rigidity, or postural instability, and response to dopaminergic therapy.

The primary method for diagnosing PD is clinical evaluation, including neurological examination and patient history. Imaging examinations include DaTscan (SPECT): Can be used to visualize dopamine transporter levels in the brain and MRI. Cerebrospinal Fluid (CSF) Alpha-Synuclein Biomarkers: Lower levels of alpha-synuclein in the CSF are potential biomarkers for PD.

One or more BTRFA or the medical head device according to the present invention may be placed on the head, in direct contact with the scalp and particularly at the back of the head of a subject, i.e., over and in close proximity of the occipital lobes, thereby allowing homogeneous and reliable exposure of the cortex of the subject to said electromagnetic waves either alone or in combination with red and near-infrared lights. Patients affected by episodes of depression may wear the medical head device daily for several consecutive months with a regimen of 1 to 2 hour per day with intervals of 7-9 h between the treatment.

The electromagnetic waves either alone or in combination with red and near-infrared lights is sufficient to cause a diminishment or elimination of tremors, resting tremor, bradykinesia, cognitive impairment, and mood disorders. It is believed that the radiation causes an upregulation of endogenous compounds in the brain, including neurotrophic factors, that serve to enhance neural growth, neurogenesis, and/or plasticity of neural function that cause the beneficial effects in the brain, and/or that the radiation results in a more normal balance of neurotransmitters in the brain. The medical head device may be used alone or in combination with Parkinson's disease medications such as carbidopa-levodopa.

Lewy Body Dementia (LBD) is a type of progressive dementia associated with the presence of Lewy bodies, e.g., accumulation of alpha-synuclein protein within neurons. It is one of the most common causes of dementia, following Alzheimer's disease and vascular dementia. These inclusions which are found in various brain regions, including the cerebral cortex, limbic system, and brainstem, disrupt the normal functioning of brain cells, particularly in areas responsible for cognition, movement, and behavior. LBD is associated with dysfunction in several neurotransmitter systems, particularly dopamine and acetylcholine. The loss of dopamine-producing neurons leads to parkinsonian symptoms, while cholinergic deficits contribute to cognitive and attentional impairments. LBD often coexists with other neuropathological features, such as amyloid plaques and neurofibrillary tangles seen in Alzheimer's disease, which complicate diagnosis and contributes to the clinical variability. LBD encompasses two related conditions: Dementia with Lewy Bodies (DLB) and Parkinson's Disease Dementia (PDD). DLB is diagnosed when cognitive and motor symptoms develop within one year of each other, or when cognitive symptoms appear first. PDD is diagnosed when motor symptoms precede cognitive decline by more than a year. Symptoms include fluctuations in attention and alertness, visual hallucinations, memory loss typically less prominent early on compared to Alzheimer's disease, as well as bradykinesia, rigidity, and resting tremor resembling Parkinson's disease. Mood disorders are common and can exacerbate cognitive symptoms. These symptoms worsened with the progression of the disease within the early, moderate and severe stages. Cognitive tests include Mini-Mental State Examination (MMSE) and Montreal Cognitive Assessment (MoCA) to assess cognitive function, and Trail Making Test or Stroop Test can help detect the attentional deficits and executive dysfunction characteristics of LBD. Cerebrospinal Fluid (CSF) biomarkers include measuring levels of alpha-synuclein, amyloid-beta, and tau proteins can help differentiate LBD from Alzheimer's disease.

Frontotemporal dementia (FTD) is a group of neurodegenerative disorders primarily affecting the frontal and temporal lobes of the brain. It is characterized by progressive changes in behavior, personality, language, and motor function. FTD is one of the most common forms of dementia in people under 65 years old, although it can also occur in older individuals. Semantic Dementia (SD) and Primary Progressive Aphasia (PPA) are subtypes or language variants of FTD, with distinct patterns of language decline. Pick's disease is another subtype of FTD characterized by the presence of Pick bodies, which are abnormal tau inclusions within neurons of the frontal and temporal lobes. These tau aggregates form the characteristic Pick bodies, which are spherical tau inclusions found in affected brain cells. These inclusions are distinct from the neurofibrillary tangles seen in Alzheimer's disease and are typically found in the swollen, ballooned neurons known as Pick cells in the cortex. The disease involves the loss of neurons and gliosis (an increase in the number of glial cells due to damage) in the affected areas of the brain, leading to the progressive atrophy of these areas and to the symptoms of the disease. Symptoms overlap with those of FTD including behavioral changes, language difficulties, and cognitive decline.

Around 30-50% of FTD cases have a familial link, often associated with mutations in genes such as MAPT (microtubule-associated protein tau), GRN (progranulin), and C9orf72 (chromosome 9 open reading frame 72). In case of FTD, degeneration of neurons in the frontal and temporal lobes, leading to atrophy in these regions is caused by Tau pathology, i.e., the abnormal accumulation of tau protein, leading to neuronal death and by TDP-43 pathology, i.e., the aggregation of TDP-43 protein (TAR DNA-binding protein 43). SD is characterized by the progressive degeneration of the anterior temporal lobes (particularly on the left side), a critical area for semantic memory, often with an accumulation of TDP-43 protein. Degeneration in these areas leads to a progressive loss of the ability to understand or produce meaningful language, a hallmark of semantic dementia. The diagnosis of Semantic Dementia is based on the consensus criteria for Frontotemporal Lobar Degeneration (FTLD), focusing on progressive loss of semantic memory and left temporal lobe atrophy on neuroimaging.

PPA specifically affects language capabilities due to degeneration in the frontal and temporal lobes. The underlying pathologies include tauopathies, TDP-43 proteinopathies, and, in some cases, Alzheimer's disease pathology. It is characterized by the gradual impairment of language function while other cognitive domains remain relatively preserved early in the disease. PPA can be classified into three main variants: non-fluent/agrammatic, semantic (overlapping with Semantic Dementia), and logopenic. Unlike other dementias, the primary impairment is in language rather than memory or behavior initially.

Symptoms of FTD may include cognitive decline with difficulty with planning, judgment, and problem-solving, often with relatively preserved memory early in the disease, as well as behavioral changes: disinhibition, apathy, lack of empathy, compulsive behaviors, and changes in eating habits. Symptoms of SD and PPA include a gradual decline in language abilities, individuals lose knowledge of word meanings, leading to difficulty naming objects (anomia) and understanding words. These dementia progress in three stages early, with mild cognitive and behavioral changes and language difficulties. The moderate or middle stage wherein symptoms become more pronounced, with increasing behavioral abnormalities, significant impact on daily functioning, and profound loss of vocabulary and understanding of words. Late stage wherein severe cognitive impairment and a complete dependence on caregivers are observed and in case of the language variant, there is a severe language impairment leads to near-total loss of communication ability. Several tests and biomarkers may be used to detect and monitor the progression of the disease. CSF biomarkers such as levels of tau protein, TDP-43 and Aβ₁₋₄₂ peptide, and thus helping to distinguish between FTD and Alzheimer's disease. Imaging biomarkers include MRI and CT showing atrophy in the frontal and temporal lobes, FDG-PET allowing to detect hypometabolism in the affected regions, and amyloid PET for detecting amyloid plaques, particularly in the case of PPA. Neuropsychological Testing for FTD allow assessing executive function, social cognition, and behavior. Speech and language tests Include the Western Aphasia Battery (WAB) and Boston Naming Test, as well as and Pyramids and Palm Trees Test assess semantic memory and naming abilities.

Cerebral amyloid angiopathy (CAA) is characterized by amyloid plaques deposit in the walls of the cerebral blood vessels, increasing the risk of vessel rupture and microbleeds. This can lead to cognitive decline and, in some cases, vascular dementia. Symptoms can include recurrent hemorrhagic strokes, cognitive impairment, and, in severe cases, dementia.

BTRFA and non-invasive medical head device as described above may be used in a method of treating and/or preventing and/or alleviating traumatic brain injury (TBI), concussions (mild TBI) and other types of neurological conditions such as brain tissue ischemia from stroke, post-stroke disorders, head injury, cerebral injury, neurological injury in a subject in need thereof, comprising administering the combination of said pulsed electromagnetic signal generated by the one or more body tissue radiofrequency applicators with said red and/or near-infrared signals generated by the one or more LEDs. The present invention thus provides a method of treating and/or preventing and/or alleviating brain injuries, such as concussions, traumatic brain injuries, post stroke disorders, in a subject in need thereof, comprising positioning the BTRFA or medical head device, at close proximity to the head of the subject, activating said one or more BTRFA in combination and simultaneously with the array of said one or more red and NIR lights, and transcranially exposing the head and cortex of the subject to a therapeutically efficient dose of pulsed electromagnetic waves or field at a specific frequency and a specific absorption rate (SAR) and to red and NIR lights or LEDs at specific wavelengths as described above.

Traumatic brain injury (hereinafter known as TB) remains as one of the leading causes of morbidity and mortality for civilians and for soldiers on the battlefield and is a major health and socio-economic problem throughout the world. The World Health Organization projected that by 2020, road traffic accidents, a major cause of traumatic brain injury, will rank third as a cause of the global burden of disease and disablement, behind only ischemic heart disease and unipolar depression. Recently, the demographics of traumatic brain injury have shifted to include more cases due to falls in middle-aged and older subjects.

Tissue damage from head injuries such as traumatic brain injury generally arises from the mechanical damage of the trauma event and subsequent secondary physiological responses to the trauma event. For example, moderate to severe traumatic brain injury can produce mechanical damage by direct trauma to brain tissue that can cause the disruption of cell membranes and blood vessels, resulting in direct and ischemic neuronal death. Then, secondary physiological responses such as inflammation and swelling can result in further damage and even death of healthy brain tissue. Importantly, even in the absence of direct mechanical injury (i.e. diffuse brain trauma), such secondary physiological responses can still occur and result in injury to healthy brain tissue. For example, astrocytes and microglia often react to head injury conditions and by secreting destructive cytokines (e.g. IL-δ, TNF-α, IFN-γ, and IL-6) as well as other inflammatory molecules, such as glutamate, reactive oxygen and nitrogen species, which, alone, or in combination, can be neurotoxic. While the primary and immediate consequences of mechanical trauma to neurons cannot be undone, secondary pathological sequelae, specifically brain swelling and inflammation, are situational candidates for intervention.

Concussions are typically mild TBI and are caused by a blow or jolt to the head or body that causes the brain to rapidly move back and forth within the skull. It may cause temporary loss of consciousness, confusion, headaches, and dizziness. Head injuries encompass any trauma to the scalp, skull, or brain. Cerebral injury refers to any damage to the brain tissue itself from lack of oxygen, trauma, stroke. This can include injuries from stroke, trauma, or lack of oxygen (hypoxia). These neurological disorders or conditions result in significant brain damage and substantial local cerebral inflammation.

One or more BTRFA or the non-invasive medical device according to the present invention may be placed in proximity and in direct contact with the injured area on the head, or in direct contact with the scalp and/or skull respectively, thereby allowing homogeneous and reliable exposure of the injured area or the entire cortex of the subject to said electromagnetic waves either alone or in combination with red and near-infrared lights. The electromagnetic waves either alone or in combination with red and near-infrared lights may promote neuro-regeneration, reduce inflammation, improve blood flow, modulate neural activity, stimulate cellular repair, reducing oxidative stress, and enhancing synaptic plasticity. In particular, the production of adenosine triphosphate (ATP), and the release of growth factors such as nerve growth factor (NGF) and brain-derived neurotrophic factor (BDNF) support the neuroregeneration, the neuroprotection, as well as the repairing of the damaged neural tissues and enhance the overall brain function. The non-invasive medical head device and/or BTRFA alone placed at proximity of a targeted area may have anti-inflammatory effects, which are critical in neurological conditions by modulating the activity of inflammatory cytokines and reducing the infiltration of inflammatory cells into the affected areas. This helps limit the extent of brain damage following a TBI, a stroke, a concussion, etc., leading to improved outcomes and faster recovery. Such treatment enhances microcirculation and increases blood flow in the brain, by promoting the dilation of blood vessels and reducing blood viscosity, thereby improving oxygen delivery to brain tissues. Restoring the blood flow to the brain is compromised, PEMF therapy can help restore circulation and oxygenation, reducing the risk of further ischemic damage and supporting the recovery of brain function. Patients may be treated non-invasive treatment daily for several consecutive months with a regimen of 5 to 30 min or 10 to 20 min per day for one or more months.

BTRFA and medical head device as described above may be used in a method of treating and/or preventing and/or reducing or eliminating depression or the symptoms of depression, delaying, reducing and/or eliminating depression and its symptoms, reducing and/or relieving migraines, reducing and/or mitigating myodesopsia and vitreous opacities, and/or tinnitus (hardness of hearing) in a subject in need thereof, comprising administering the combination of said pulsed electromagnetic signal generated by the one or more body tissue radiofrequency applicators with said red and/or near-infrared signals generated by the one or more LEDs. The present invention thus provides a method of treating and/or preventing and/or mitigating the symptoms and/or relieving depression, migraines, myodesopsia, and/or tinnitus in a subject in need thereof, comprising positioning said one or more BTRFA or medical head device, at close proximity to the head of the subject, activating said one or more BTRFA in combination and simultaneously with the array of said one or more red and NIR lights, and transcranially exposing the head and cortex of the subject to a therapeutically efficient dose of pulsed electromagnetic waves or field at a specific frequency and a specific absorption rate (SAR) and to red and NIR lights or LEDs at specific wavelengths as described above.

20%-25% of people suffer an episode of depression at some point during their lifetimes. The disease affects people of all ages, including children, adults, and the elderly. There are several types of depression which vary in severity and average episode length. Two of the most common types are major depression and chronic depression. Chronic depression is generally a less severe form of depression, having milder but longer lasting symptoms than major depression. The symptoms of both types of depression are essentially the same, and include sadness, loss of energy, hopelessness, difficulty concentrating, insomnia, and irritability. Individuals suffering depression are also more likely to engage in drug or alcohol abuse, and if untreated, depression can lead to violence, including suicide.

One or more BTRFA or the medical head device according to the present invention may be placed on the head, in direct contact with the scalp and/or skull of a subject, thereby allowing homogeneous and reliable exposure of the cortex of the subject to said electromagnetic waves either alone or in combination with red and near-infrared lights. Patients affected by episodes of depression may wear the medical head device daily for several consecutive months with a regimen of 5 to 30 min or 10 to 20 min per day. The electromagnetic waves either alone or in combination with red and near-infrared lights is sufficient to cause a diminishment or elimination of depression and its symptoms, and/or delays, reduces, or eliminates the onset of depression or depressive symptoms. It is believed that the radiation causes an upregulation of endogenous compounds in the brain, including neurotrophic factors, that serve to enhance neural growth, neurogenesis, and/or plasticity of neural function that cause the beneficial effects in the brain, and/or that the radiation results in a more normal balance of neurotransmitters in the brain. The medical head device may be used alone or in combination with antidepressant medications presently available, including tricyclic antidepressants (TCAs), monoamine oxidase inhibitors (MAOIs), selective serotonin reuptake inhibitors (SSRIs), and selective norepinephrine reuptake inhibitors (SSNRIs).

Migraines and migraine episodes can be debilitating and can affect patient quality of life and productivity. The migraine episode may last for up to 2 to 3 days and may require medical attention in at least some instances. Although many people associate migraine episodes with headaches, a migraine episode can include four phases: a premonitory phase also referred to as “pre-headache”; an aura phase which may include visual symptoms such as flashes of lights or formations of dazzling lines, blurred or shimmering or cloudy vision, and tunnel vision or non-visual symptoms such as auditory and/or olfactory hallucinations, vertigo, tingling, and hypersensitivity to touch; a headache phase; and a postdrome phase with symptoms of fatigue, poor concentration, poor comprehension, and/or lowered intellect level. In some migraine patients, the disorder can manifest daily, and when occurring on fifteen days or more a month can be called chronic migraine.

Patients affected by migraines, headaches or migraine episodes may wear the medical head device at the apparition of the first symptoms (for example during the pre-headache phase) or during a migraine attack during 5, 10, 15, 20, 25 or 30 min depending on the intensity of the pain and may repeat the same administration 7-8 hours thereafter. The medical head device may be used alone or in combination with pharmaceutical treatments.

Tinnitus is a symptom that is perceived as a continuous or intermittent noise in the ear even when there is no external sound source, and the patient is suffering from headache, sleep disorders, nervous breakdowns, depression, etc. Tinnitus has a large individual difference and various causes. Most common cause is a decrease in function due to aging of the sensory nerve system; a kind of neuro-electrical spurious generated by the cochlea hair cell damage or degeneration caused by disease, aging, fatigue, mental anxiety and stress, as well as certain brain neurological diseases.

BTRFA or the medical head device according to the present invention are particularly active for tinnitus caused by fatigue and it may be placed at proximity or as to cover the auditory organs tissues during 5 to 30 min once or twice per day with at least 7 hours interval for synergistically stabilizing and/or relieving the symptoms of tinnitus and/or of the hearing loss.

With aging and eye-fatigue are often associated with myodesopsia which is caused by the formation of other collagen-based structures in the form of light scattering opacities responsible for the phenomenon of floaters, eye floaters, or vitreous floaters. These collagenous aggregates scatter light and cast shadows on the retina, which are perceived by the patient as grey objects of different sizes and shapes. Although myodesopsia was previously not considered a serious problem in ophthalmology, many patients with symptomatic vitreous floaters experience a significantly negative impact on their quality of life. In terms of vision, studies have shown that while there can be loss of visual acuity, there is significant degradation in contrast with the sensitivity function, which likely accounts for profound unhappiness in some cases.

BTRFA or the medical head device according to the present invention are particularly active for myodesopsia caused by eye fatigue and it may be placed at proximity to the frontal lobes or the fronto-temporal lobes or the whole head during 5 to 30 min once or twice per day with at least 7 hours interval for synergistically stabilizing and/or relieving the symptoms of myodesopsia and eye fatigue.

Finally, BTRFA and non-invasive head device as described above may be used by healthy patients for general wellness purposes, particularly for relieving headaches and signs of fatigues, and/or enhancing brain activity and cognitive abilities, comprising administering the combination of said pulsed electromagnetic signal generated by the one or more body tissue radiofrequency applicators with said red and/or near-infrared signals generated by the one or more LEDs. The present invention thus provides a method of improving general wellness purposes, particularly for relieving headaches and signs of fatigues, and/or enhancing general mental and cognitive abilities in a healthy subject, comprising positioning the BTRFA or medical head device, at close proximity to the head of the subject, activating said one or more BTRFA in combination and simultaneously with the array of said one or more red and NIR lights, and transcranially exposing the head and cortex of the subject to an efficient dose of pulsed electromagnetic waves or field at a specific frequency and a specific absorption rate (SAR) and to red and NIR lights or LEDs at specific wavelengths as described above.

The ability of the brain to be stimulated and enhanced is well recognized as brain plasticity and is a fundamental property of the brain nervous system. The non-invasive head device according to the present invention is a valuable tool for maintaining overall health and well-being, as it may be beneficial to improve blood flow, reduce inflammation, and support cellular energy production.

It may be beneficial to alleviate headaches by improving blood circulation, reducing inflammation, and modulating neural activity. By enhancing blood flow, more oxygen and nutrients are delivered to the brain, which can reduce headache intensity and frequency.

The use of non-invasive head devices may also be useful to reduce the signs of fatigue—which may be due to a variety of factors, including poor circulation, and inadequate cellular energy production—helping combating fatigue by enhancing cellular energy production (ATP) and improving microcirculation, which can boost energy levels and reduce feelings of tiredness. Finally, it may enhance cognitive function by promoting neurogenesis and synaptic plasticity, improving blood flow to the brain, and reducing oxidative stress, and thus increasing cognitive performance, improving focus & concentration, enhancing memory, reducing anxiety and improving the overall mental clarity.

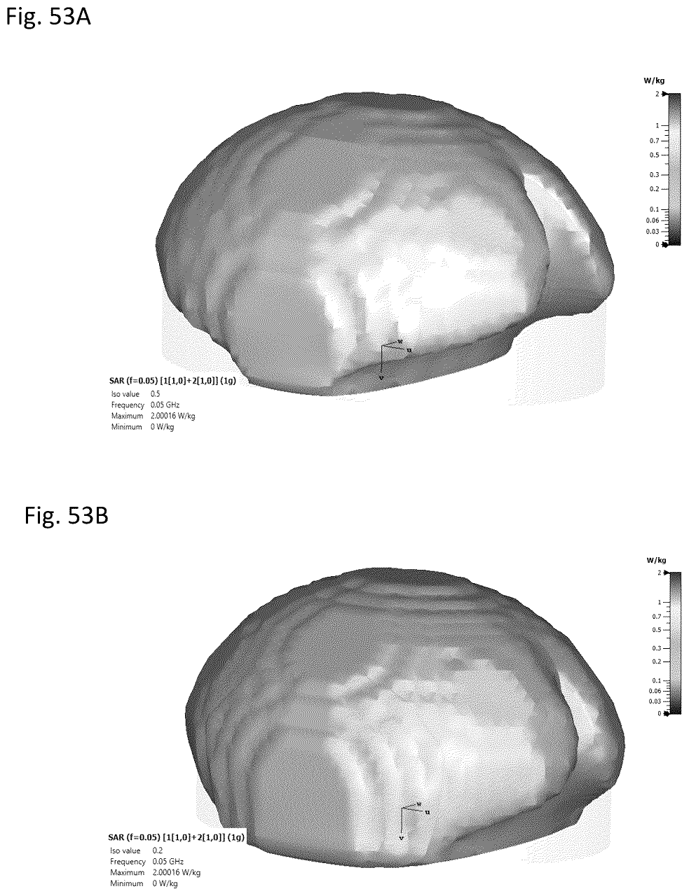

The non-invasive medical device according to the present invention is present in the form of a hat or augmented hat (FIG. 53) which the patient may wear a few times per week or daily for 10 to 20 min depending on the sought effect, without any constraints, feeling of pain or heat, while continuing his daily routine. Regular use thereof can contribute to a more energized, focused, and balanced state of mind and body, making it an appealing option for those seeking to enhance their quality of life.

### EXAMPLES

### Example 1: X-BTRFA

### Total Wire or PCB Length and Resonant Frequency Variation

For X-BTRFA (130), the total length of the wire or PCB in each branch should be approximately twice as long as in the case of Z-BTRFA (140) or roughly one wavelength at the desired operating frequency. For reference, this corresponds to:
≈330 mm at 915 MHz,
≈6000 mm at 50 MHz,
≈3000 mm at 100 MHz.

The resonant frequency of the X-BTRFA (130) may shift when the applicator is placed in proximity to a large dielectric object with a high relative dielectric constant (relative dielectric permittivity), typically ranging from 45 to 55 for biological tissue.

### Reflection and Matching Characteristics in Free Space

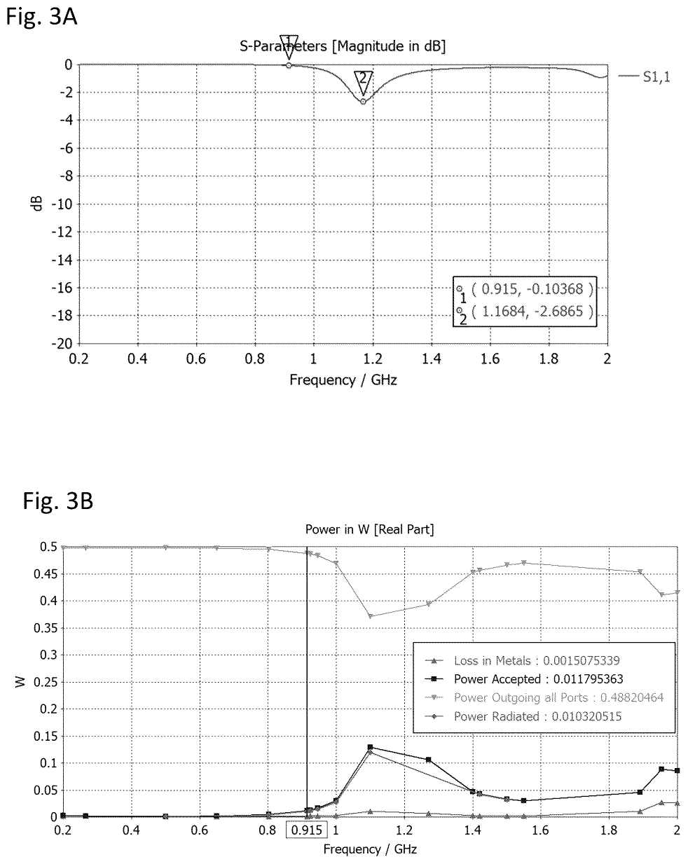

When the X-BTRFA (130) was tested in free space (air) at 915 MHz, the S11 parameter (scattering parameter or return loss—RL) graph in FIG. 3A exhibited:
High reflection across nearly the entire observed frequency range,
Poor impedance matching at 1186 MHz, with an RL of −2.6865 dB,
Near-total reflection at 915 MHz, with an RL of −0.10368 dB.

These findings indicate that in free-space conditions, the X-BTRFA (130) experiences substantial signal reflection, limiting its efficiency when not placed near a dielectric medium.

### Power Distribution Analysis in Free Space

The power distribution graph of the X-BTRFA (130) in free space, as shown in FIG. 3B, reveals that at 915 MHz (marked by a vertical line):
Only 2.36% (0.011795/0.5) of the total available power (0.5 W) was accepted by the X-BTRFA (130).
Over 97.5% (0.488204/0.5) of the power was reflected back to the RF generator.
Total dielectric loss was negligible at 3.3196×10⁻⁵/0.5.
Metal loss was minimal, at 0.3% (0.0015/0.5).
Radiated power accounted for 2% (0.01032/0.5).

These results indicate that X-BTRFA (130) exhibits low efficiency in free space, primarily due to poor impedance matching, causing most of the energy to be reflected.

### Radiation Characteristics in Free Space

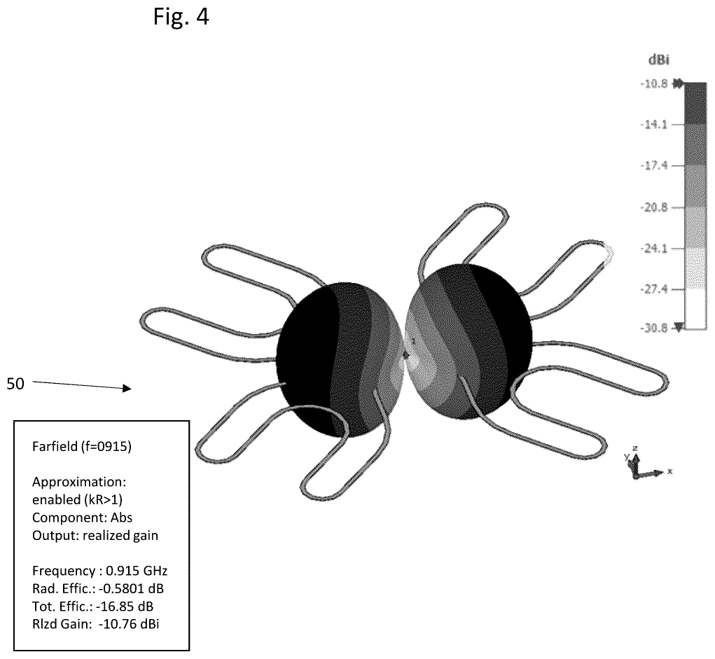

In a free-space environment, the X-BTRFA (130) behaves as a bi-directional antenna, with two major radiation beams oriented in opposite directions, parallel to the applicator plane.
The maximum directivity in both beams was measured at approximately 6 dBi.
However, due to the significant reflection at 915 MHz, the maximum realized gain was only −10.8 dBi, as illustrated in FIG. 4.

### Performance in the Presence of Body Tissue

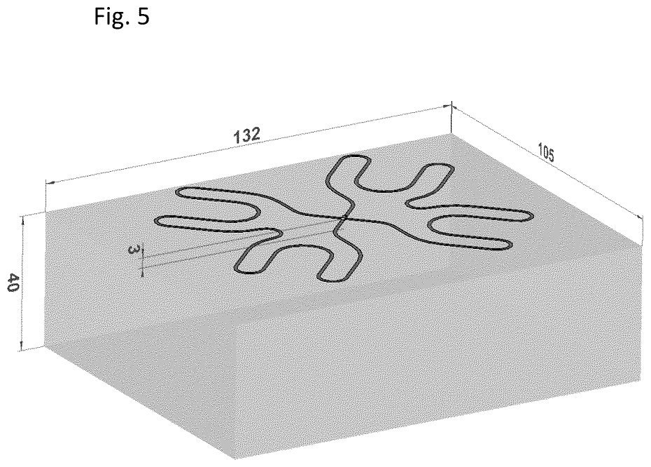

When the X-BTRFA (130) was placed in proximity to a large sample of body tissue, as depicted in FIG. 5, the resonant frequency shifted downward to 915 MHz.

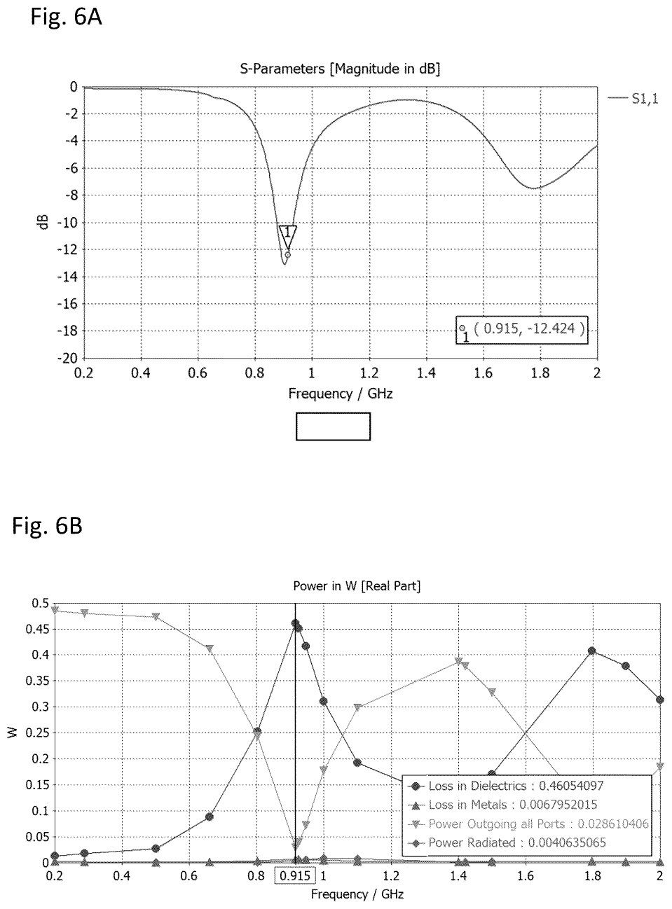

For an exemplary body tissue sample of 132×105×40 mm, with a 3 mm separation distance between the X-BTRFA and the tissue surface, the reflection coefficient (S11 parameter) at 915 MHz was significantly improved, reaching −12.424 dB, as shown in FIG. 6A.

The exact resonant frequency of X-BTRFA (130) can be fine-tuned based on the distance between the applicator and the tissue surface, offering adjustability for various application scenarios.

### Power Distribution Analysis Near Body Tissue

As illustrated in FIG. 6B, when the X-BTRFA (130) was positioned near body tissue, the power acceptance efficiency drastically improved at 915 MHz:
94.27% (0.471394/0.5) of the total available power (0.5 W) was accepted by the X-BTRFA (130).
Only 5.7% (0.02861/0.5) of the power was reflected back to the RF generator.
Approximately 92.1% (0.46054/0.5) of the power accepted by the X-BTRFA was effectively delivered to the body tissue.
Metal loss accounted for approximately 1.358% (0.00679/0.5).
Only 0.8127% (0.004064/0.5) of the power was radiated into free space.

These findings demonstrate that X-BTRFA (130) operates optimally when coupled with biological tissue, ensuring efficient RF energy absorption with minimal losses.

### Specific Absorption Rate (SAR) Distribution

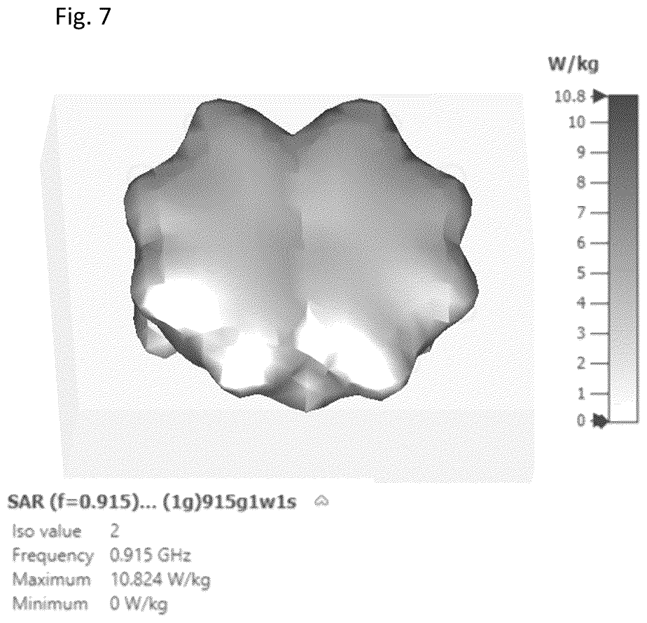

As illustrated in FIG. 7, the X-BTRFA (130) facilitated an SAR distribution pattern where the exposed body tissue regions absorbed power levels exceeding 2 W/kg.

Furthermore, the maximum SAR value in this setup was measured at 10.8 W/kg, indicating that the RF energy was evenly and effectively distributed across the targeted biological tissue area.

This widespread and uniform SAR distribution demonstrates the effectiveness of X-BTRFA (130) for targeted RF energy applications, ensuring controlled and efficient power absorption within the exposed biological tissue.

### Example 2: Y-BTRFA

### Reflection and Matching Characteristics in Free Space

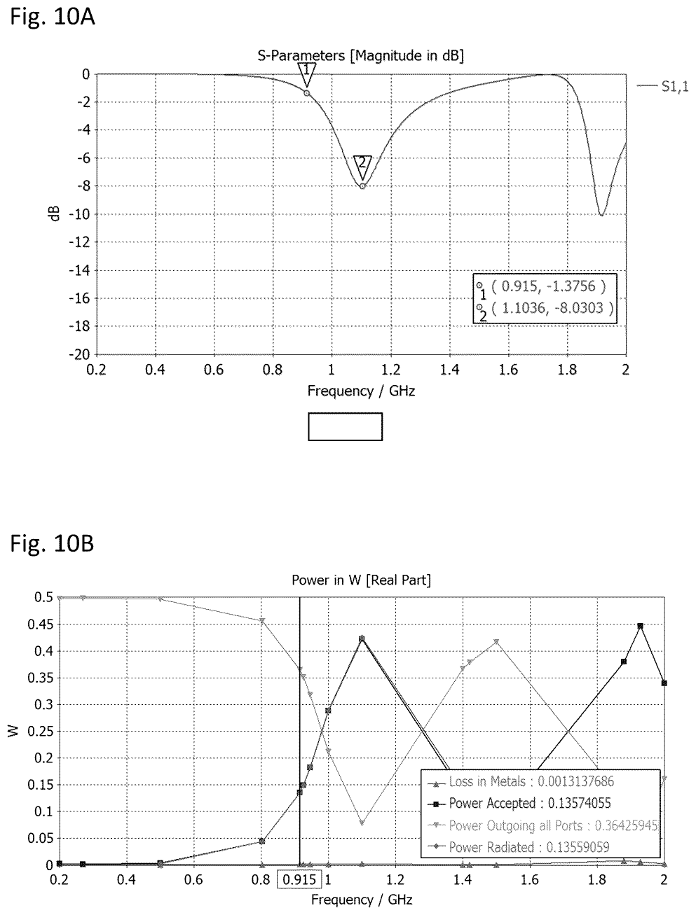

When the Y-BTRFA (120) was tested in free space (air) at 915 MHz, the S11 parameter (scattering parameter or return loss—RL) graph in FIG. 10A exhibited:
High reflection at 915 MHz, with an RL value of −1.3756 dB, indicating poor impedance matching.
Moderate impedance matching at 1103 MHz, with an RL value of −8.0303 dB.

These results suggest that in free-space conditions, the Y-BTRFA (120) suffers from substantial signal reflection at 915 MHz, limiting its efficiency at this frequency.

### Power Distribution Analysis in Free Space

As shown in FIG. 10B, at 915 MHz (marked with a vertical line), the Y-BTRFA (120) placed in free space exhibited the following power characteristics:
27.15% (0.1357405/0.5) of the total available power (0.5 W) was accepted by the Y-BTRFA (120).
More than 72% (0.364/0.5) of the power was reflected back to the RF generator due to poor impedance matching.
Total dielectric loss was negligible, measured at 3.1141×10⁻⁵/0.5.
Metal loss was minimal, accounting for only 0.26% (0.001313/0.5).
Radiated power constituted approximately 27% (0.13559/0.5) of the total available power.

These findings confirm that the Y-BTRFA (120) in free space behaves as a highly reflective antenna, with a significant portion of the input power being reflected rather than efficiently utilized.

### Radiation Characteristics in Free Space

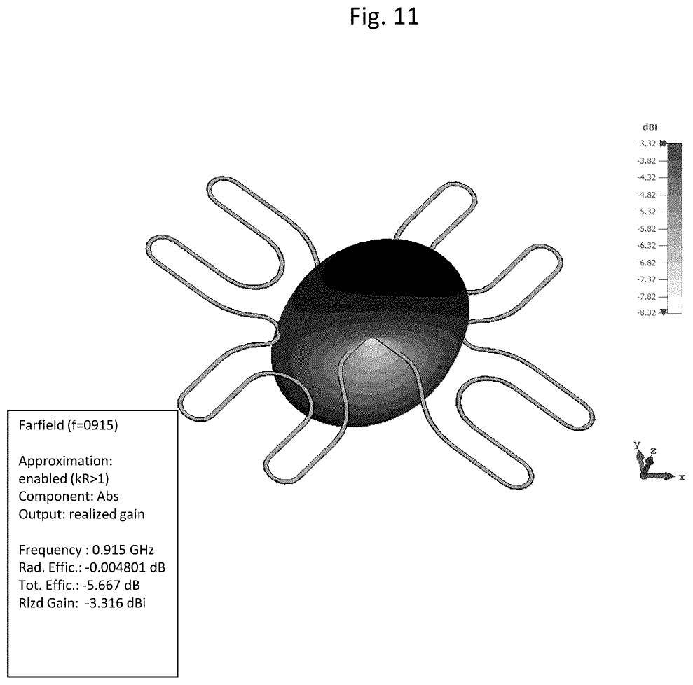

When placed in free space, the Y-BTRFA (120) functioned as a bi-directional antenna, with two primary radiation beams oriented in opposite directions, normal to the applicator plane.
Under ideal impedance matching conditions, the maximum achievable gain in both beams was approximately 2.48 dBi.
However, due to the significant reflection at 915 MHz, the actual realized gain was only 3.32 dBi, as depicted in FIG. 11.

This behavior indicates that Y-BTRFA (120) does not operate efficiently in free space, as most of the input power is reflected rather than radiated effectively.

### Performance Improvement in the Presence of Body Tissue

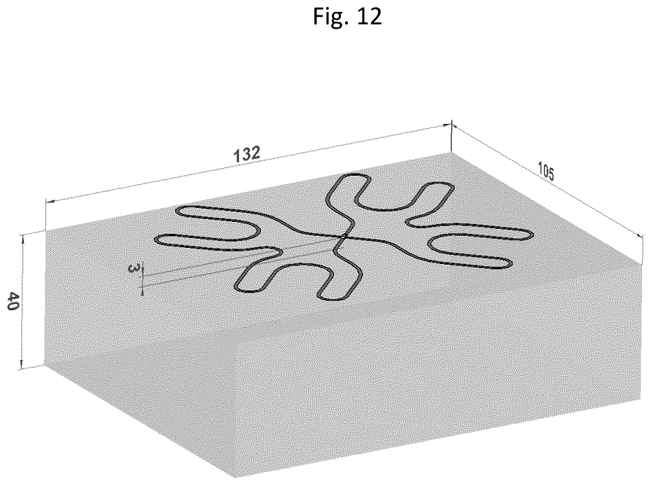

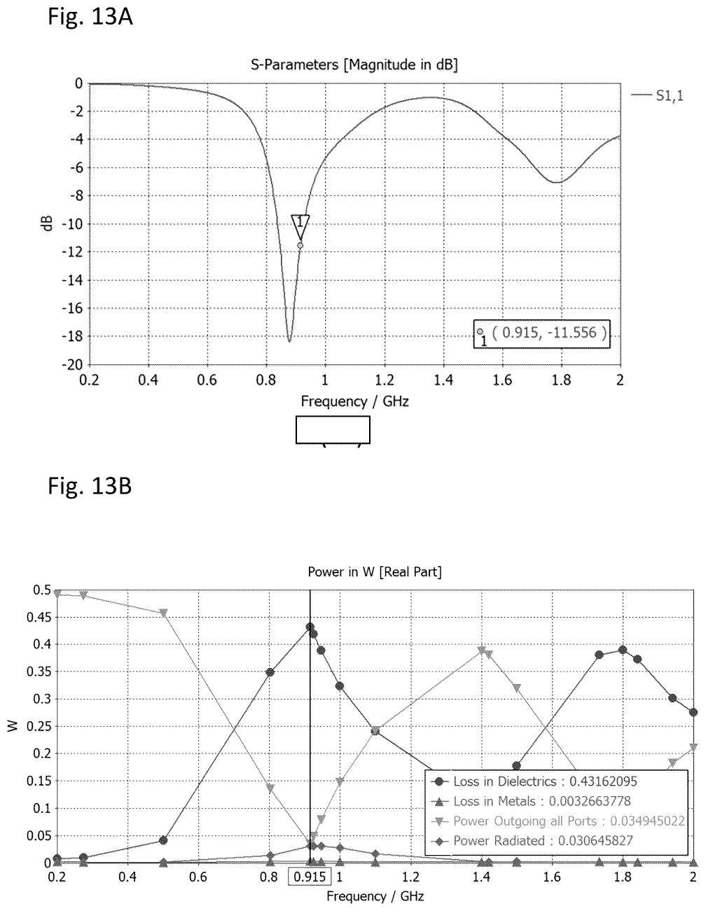

When the Y-BTRFA (120) was placed in proximity to a large biological tissue sample, as illustrated in FIG. 12, the resonant frequency shifted downward to 898 MHz.
The reflection coefficient (S11 parameter) significantly improved, achieving an RL value of −18 dB, as shown in FIG. 13A.
This represents a substantial enhancement in impedance matching compared to the free-space case in FIG. 10A.
The exact resonant frequency of the Y-BTRFA (120) depends on the distance between the X-BTRFA (130) and the tissue surface, allowing for fine-tuning based on the application requirements.

### Power Distribution Analysis Near Body Tissue

The power distribution graph at 915 MHz (marked with a vertical line) in FIG. 13B indicates that, when placed close to body tissue, the Y-BTRFA (120) exhibited significantly improved power absorption:
93% of the total available power (0.5 W) was accepted by the Y-BTRFA (120).
Only 7% (0.03494/0.5) of the power was reflected back to the RF generator.
Approximately 86.3% (0.4316/0.5) of the power accepted by the Y-BTRFA (120) was effectively delivered to the body tissue, demonstrating its high efficiency in a tissue-coupled environment.
Metal loss accounted for 0.6% (0.003266/0.5), indicating minimal energy dissipation in the antenna structure.
Radiated power was measured at approximately 6.12% (0.0306/0.5), contributing to minor free-space emissions.

These results highlight the importance of proper impedance matching and tissue proximity, as Y-BTRFA (120) performs optimally when operating in direct contact with a biological medium.

### Specific Absorption Rate (SAR) Distribution

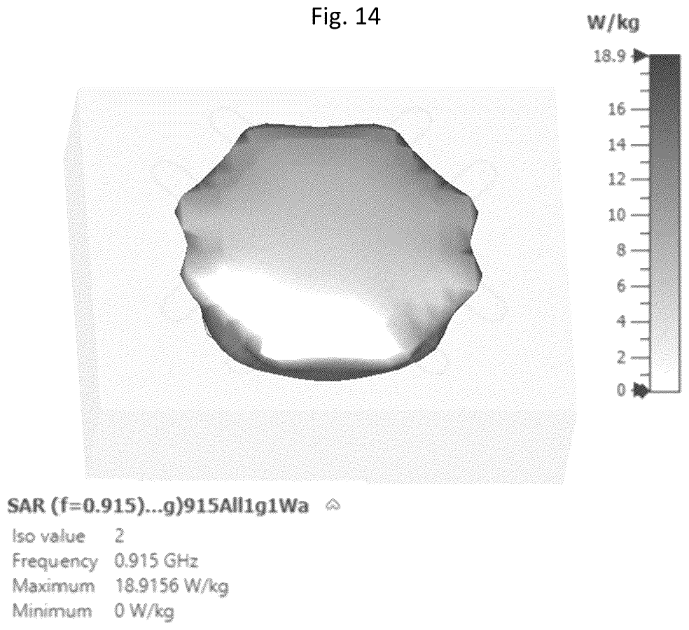

As shown in FIG. 14B, the Y-BTRFA (120) exhibited a distinctive SAR distribution, ensuring effective RF energy absorption by biological tissue.
The SAR within the exposed tissue region exceeded 2 W/kg, demonstrating strong RF energy coupling.
The maximum SAR measured in this setup was 18.9 W/kg, indicating that the RF energy was efficiently delivered and evenly distributed within the exposed area.

This SAR profile confirms that Y-BTRFA (120) is highly effective for localized RF energy delivery, making it suitable for biomedical and therapeutic applications where precise RF exposure is required.

### Example 3: Z-BTRFA

### Reflection and Impedance Matching Characteristics in Free Space

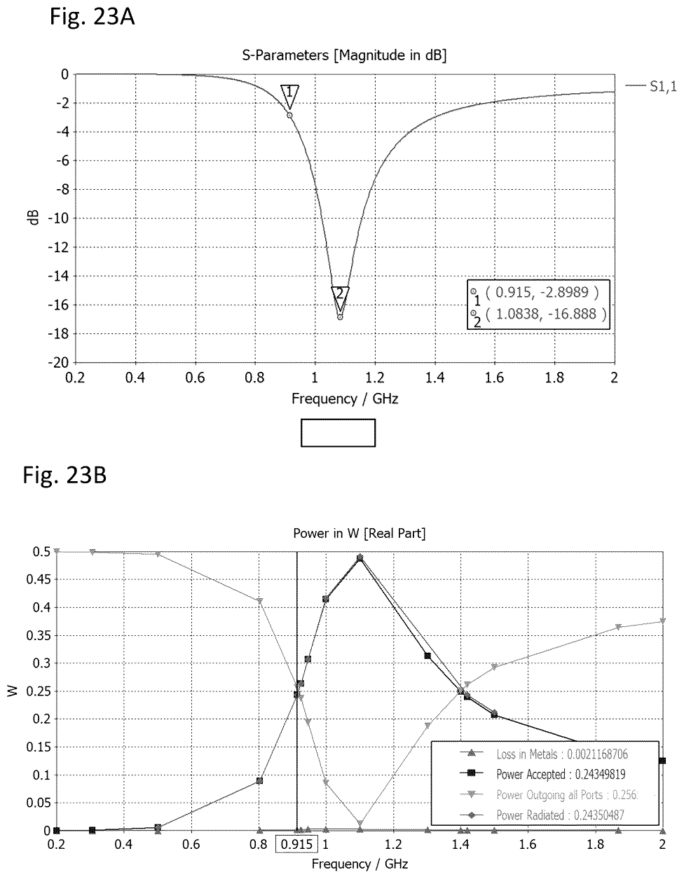

When the Z-BTRFA (140) was tested in free space (air) at 915 MHz, the S11 parameter (scattering parameter or return loss—RL) graph in FIG. 23A exhibited:
Moderate reflection at 915 MHz, with an RL value of −2.89 dB, indicating partial impedance matching.
Excellent impedance matching at 1083.8 MHz, with an RL value of −16.888 dB, demonstrating low reflection and improved efficiency at this frequency.

These results suggest that in free-space conditions, the Z-BTRFA (140) exhibits moderate efficiency at 915 MHz but operates more optimally at a higher resonant frequency of 1083.8 MHz.

### Power Distribution Analysis in Free Space

As shown in FIG. 23B, at 915 MHz (marked with a vertical line), the Z-BTRFA (140) placed in free space exhibited the following power characteristics:
48.7% (0.2434/0.5) of the total available power (0.5 W) was accepted by the Z-BTRFA (140).
51.2% (0.2435/0.5) of the power was reflected back to the RF generator due to moderate impedance mismatch.
Total dielectric loss was negligible, as the only dielectric present in the analyzed model was the Teflon (PTFE—Polytetrafluoroethylene) insulator within the coaxial cable.
Metal loss was minimal, accounting for 0.42% (0.002117/0.5).
Radiated power constituted approximately 48.7% (0.2435/0.5) of the total available power, indicating strong radiation characteristics in free-space conditions.

These findings confirm that the Z-BTRFA (140) exhibits higher efficiency compared to other configurations in free space, making it a better standalone RF applicator when not placed in direct contact with a dielectric medium.

### Radiation Characteristics in Free Space

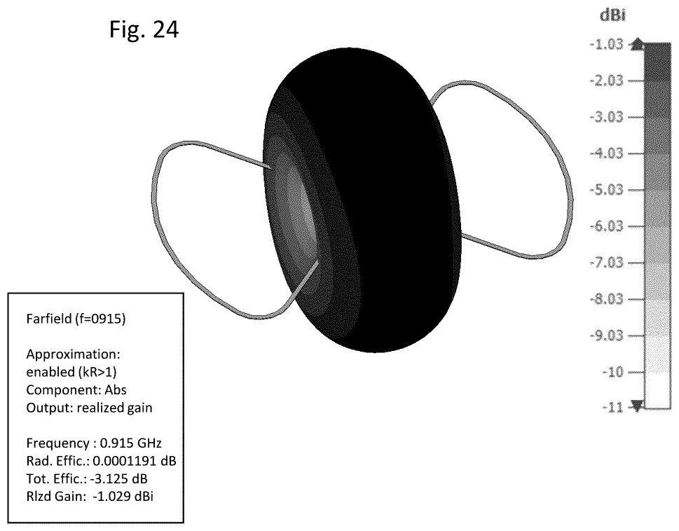

When tested in free-space conditions, the Z-BTRFA (140) exhibited an omni-directional radiation pattern, forming a doughnut-shaped radiation profile.
Under ideal impedance matching conditions, the maximum omnidirectional gain was approximately 2 dBi.
However, due to the −3 dB reflection at 915 MHz, the maximum realized gain was reduced to 1 dBi, as illustrated in FIG. 24.

These findings suggest that the Z-BTRFA (140) effectively radiates power omnidirectionally but requires impedance tuning for optimal efficiency at 915 MHz.

### Performance Improvement in the Presence of Body Tissue

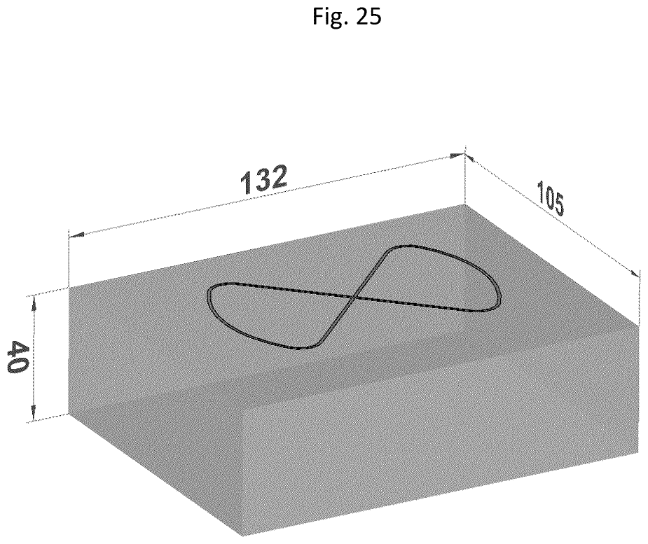

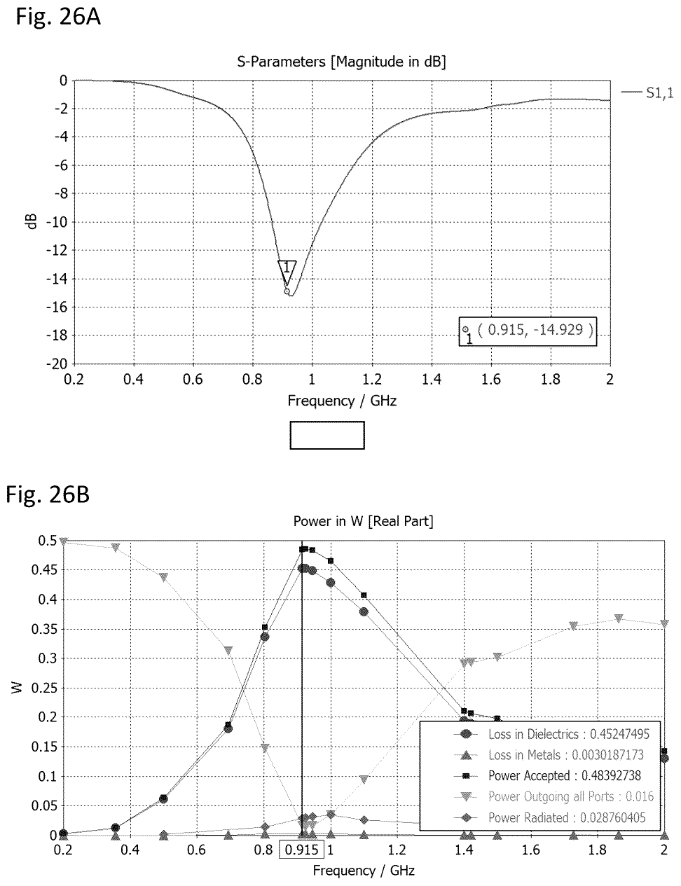

When the Z-BTRFA (140) was placed in proximity to a large biological tissue sample, as depicted in FIG. 25, the resonant frequency shifted downward to 915 MHz.
The reflection coefficient (S11 parameter) significantly improved, reaching an RL value of −14.929 dB, as shown in FIG. 26A.
This represents a substantial enhancement in impedance matching compared to the free-space case shown in FIG. 23A.
The exact resonant frequency of the Z-BTRFA (140) depends on the distance between the applicator and the tissue surface, allowing for fine-tuning to optimize performance based on application-specific requirements.

### Power Distribution Analysis Near Body Tissue

The power distribution graph at 915 MHz (marked with a vertical line) in FIG. 26B indicates that, when placed close to body tissue, the Z-BTRFA (140) exhibited significantly improved power absorption:
96.78% (0.48392/0.5) of the total available power (0.5 W) was accepted by the Z-BTRFA (140).
Only 3.2% (0.016/0.5) of the power was reflected back to the RF generator, demonstrating excellent impedance matching in tissue-coupled conditions.
Approximately 90.49% (0.452/0.5) of the power accepted by the Z-BTRFA (140) was effectively delivered to the body tissue, demonstrating high RF energy coupling efficiency.
Metal loss accounted for only 0.6% (0.003018/0.5), indicating minimal energy dissipation within the applicator.
Radiated power was measured at approximately 5.75% (0.02876/0.5), contributing to minor free-space emissions.

These results highlight the significant improvement in RF power utilization when the Z-BTRFA (140) is coupled with biological tissue, confirming its superior performance as a body-tissue applicator compared to its free-space operation.

### Specific Absorption Rate (SAR) Distribution

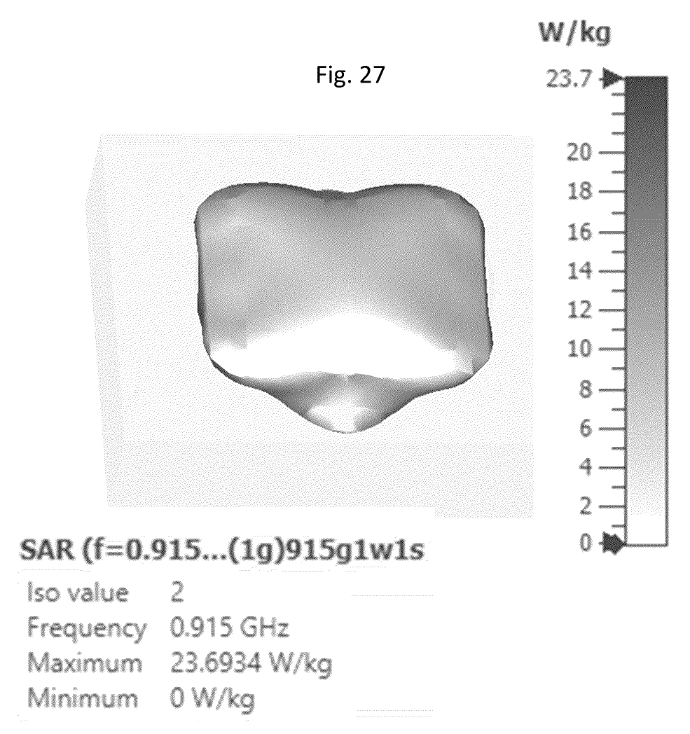

As illustrated in FIG. 27, the Z-BTRFA (140) exhibited a distinctive SAR distribution, ensuring efficient RF energy absorption by biological tissue.
The SAR within the exposed tissue region exceeded 2 W/kg, confirming its ability to deliver RF energy effectively to biological tissue.
The maximum SAR measured in this setup was 23.7 W/kg, indicating that the RF energy was efficiently delivered and distributed within the exposed area.

This SAR profile demonstrates that the Z-BTRFA (140) is highly effective for localized RF energy applications, making it suitable for therapeutic, medical, and diagnostic applications requiring precise RF exposure.

### Example 4: Comparative Power Distribution

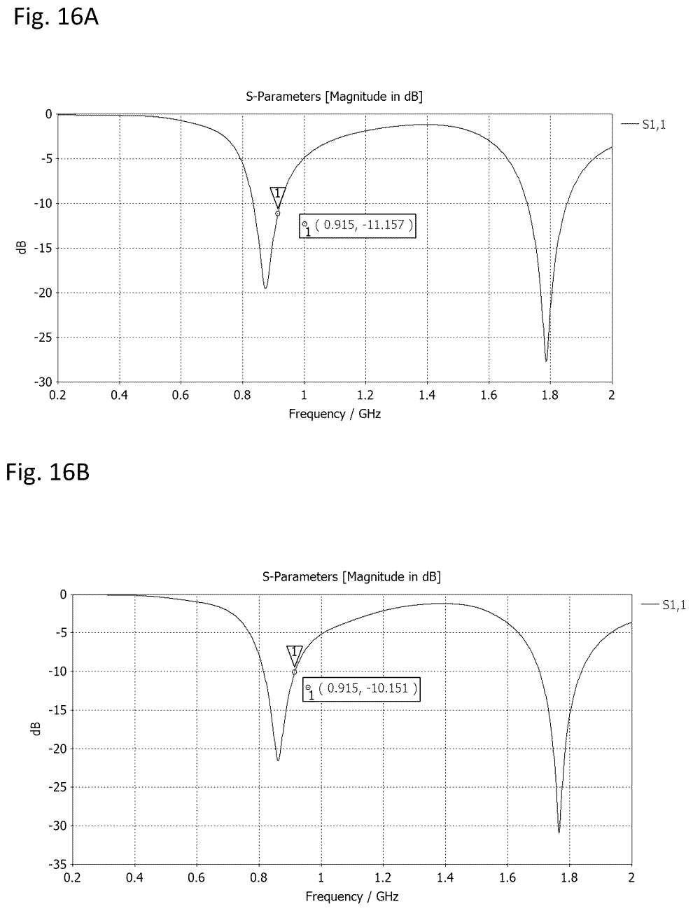

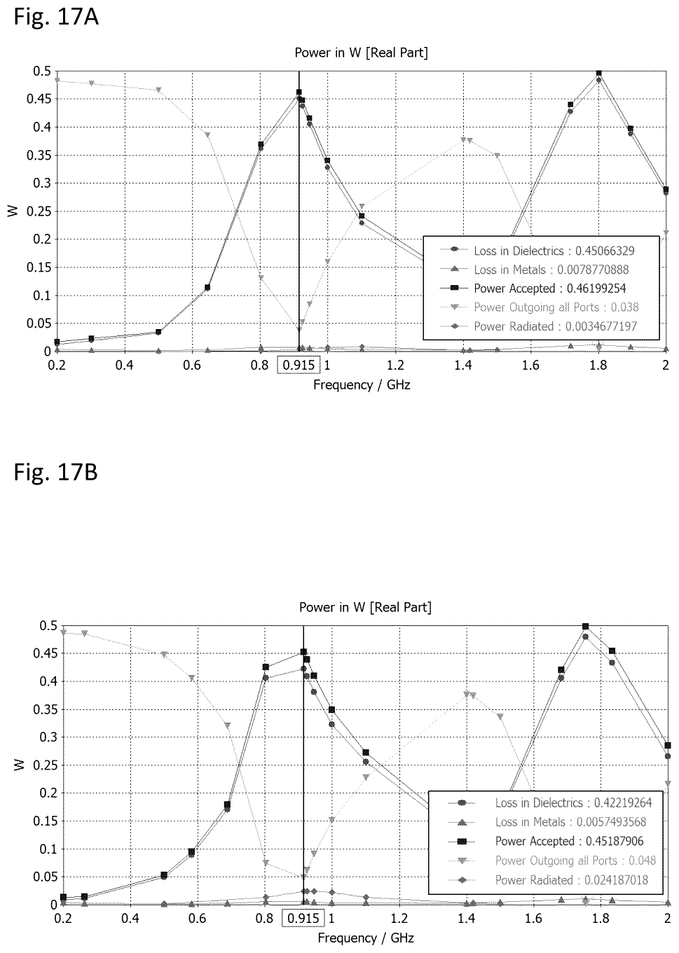

A comparative analysis of printed Body Tissue Radiofrequency Applicators (BTRFAs) was conducted, evaluating the simulated S11 parameters for both the printed X-BTRFA (130) and printed Y-BTRFA (120), as depicted in FIGS. 16A and 16B. Additionally, a power distribution comparison was performed between the printed X-BTRFA (130) and printed Y-BTRFA (120) (FIGS. 17A and 17B), which yielded results consistent with those obtained for their corresponding wire-based models.

The printed X-BTRFA (130) exhibited an acceptance of 92.4% of the total available power (0.5 W), equivalent to 0.46199 W, while 7.6% (0.038 W) was reflected back to the RF generator. Of the accepted power, approximately 90.132% was successfully delivered to the body tissue (FIG. 17A). The metal loss increased to 1.575%, compared to 1.358% in the corresponding wire model, which was anticipated due to the significantly thinner printed metal conductors. The radiated power experienced a slight reduction to 0.694%, from the previous 0.8127% observed in the wire model.

The printed Y-BTRFA (120) demonstrated an acceptance of 90.36% (0.4518 W) of the total available power (0.5 W), with 9.6% (0.048 W) being reflected back to the RF generator. Of the accepted power, approximately 84.439% was transmitted to the body tissue (FIG. 17B). The metal loss increased to 1.15%, from 0.64% in the corresponding wire model, which was expected due to the thinner printed metal elements. The radiated power showed a minor decrease to 4.8%, down from the previous 5.4% (0.027 W of 0.5 W).

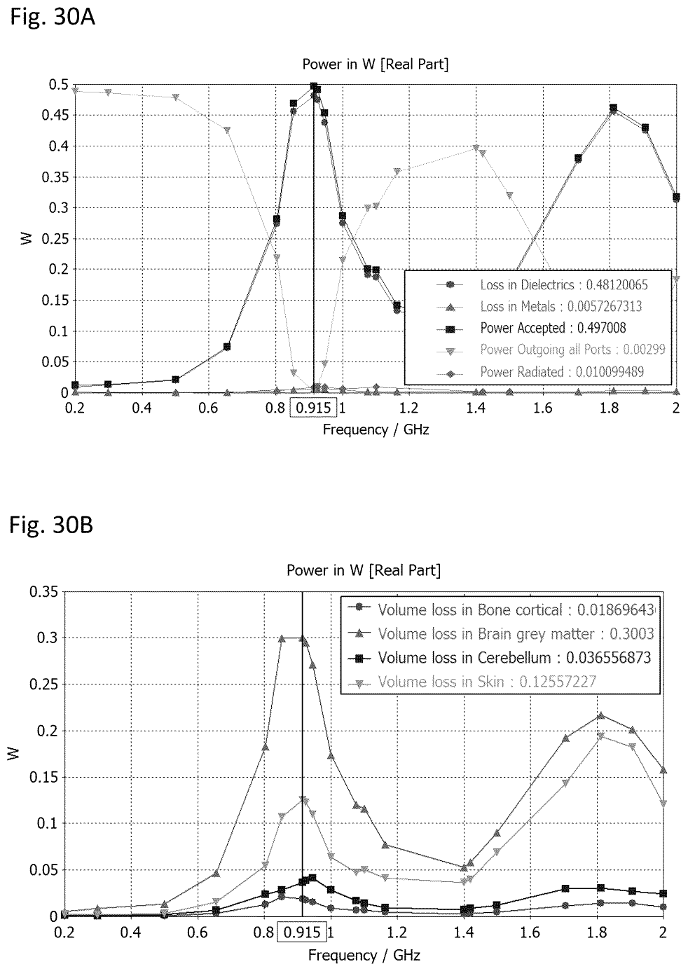

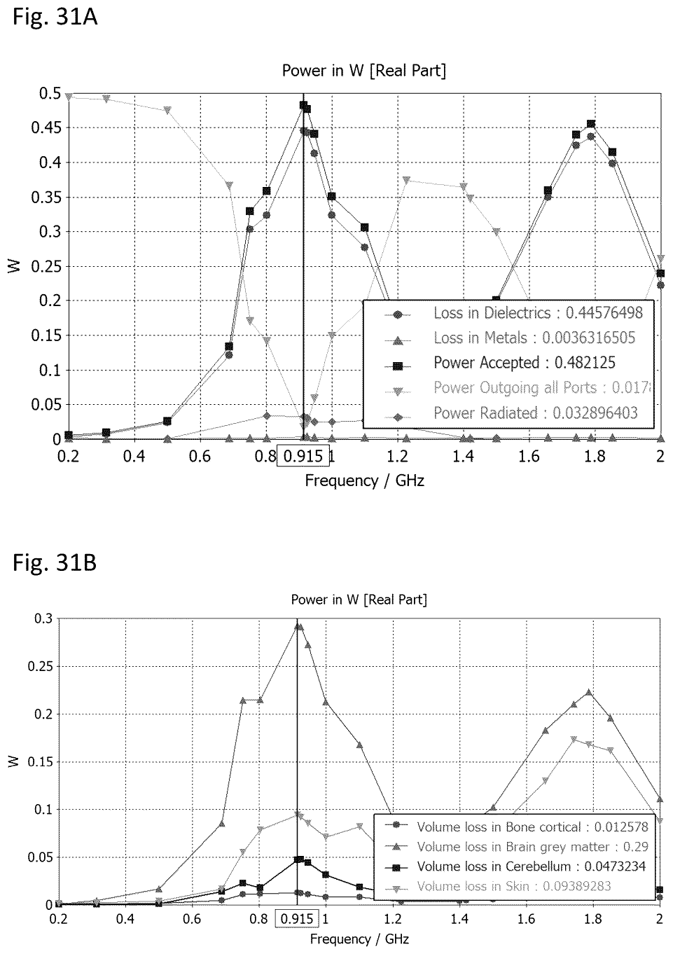

A power distribution comparison was further conducted between the X-BTRFA (130) (FIG. 30A) and Y-BTRFA (120) (FIG. 31A) when placed in proximity to multilayer body tissue. At 915 MHz (marked with a vertical line), X-BTRFA (130) accepted 99.4% (0.497 W) of the total available power (0.5 W), with only 0.6% (0.00299 W) being reflected back to the RF generator. Of the accepted power, 96.2% (0.481 W) was delivered to the body tissue. The metal loss accounted for 1.14% (0.005726 W), while approximately 2% (0.01 W) was radiated into free space. At 915 MHz (marked with a vertical line in FIG. 31A), Y-BTRFA (120) accepted 96.4% (0.482 W) of the total available power (0.5 W), with 3.4% (0.017 W) reflected back to the RF generator. Of the accepted power, 89.1% (0.445 W) was successfully delivered to the body tissue. The metal loss was approximately 0.726% (0.00363 W), while 6.56% (0.0328 W) was radiated into free space.

Both X-BTRFA (130) and Y-BTRFA (120) demonstrated high efficiency, with 96.2% and 89.1% of RF energy, respectively, being successfully transferred to the body tissue.

The power distribution across different tissue layers in the head was also analyzed using X-BTRFA (130) and Y-BTRFA (120). At 915 MHz (marked with a vertical line), the power delivery was as follows:
FIG. 30B (X-BTRFA 130):
   60% (0.300 W) to the Brain,
   25% (0.125 W) to the Skin,
   7.3% (0.03655 W) to the Cerebrospinal Fluid (CSF),
   3.6% (0.0186 W) to the Bone/Skull.
FIG. 31B (Y-BTRFA 120):
   58% (0.29 W) to the Brain,
   18.8% (0.0938 W) to the Skin,
   9.46% (0.0473 W) to the CSF,
   2.5% (0.0125 W) to the Bone/Skull.

FIGS. 30B and 31B further highlighted an interesting observation regarding power distribution at the second resonance frequency (1.8 GHz). At 1.8 GHz, the efficiency of power transfer to body tissue remained nearly equivalent to that at 915 MHz. However, a notable shift in distribution occurred:
The power delivered to the skin increased,
The power absorbed by the Brain decreased to 39-42%, reflecting a 10-15% reduction compared to 915 MHz.

These results were consistent with the general characteristics of RF waves, where penetration depth is inversely proportional to frequency. These findings further demonstrated that the BTRFA design exhibits optimal performance at operating frequencies around 915 MHz.

### Example 5: Pilot Clinical Phase for Testing Safety and Efficacy of the Medical Head Device on Alzheimer's Disease

### Example 5.1: Enrollment of Patients

Device is intended for adults of 65 years and older, who have been diagnosed with mild or moderate stage of Alzheimer's Disease, according to the National Institute of Neurological and Communicative Disorders and Stroke-Alzheimer's Disease and Related Disorders Association (NINCDS-ADRDA) criteria, and who are scoring between 16-26 at the Mini-Mental State Examination (MMSE).

In addition, blood brain biomarkers (BBM), such as Aβ42, Aβ40, and tau are used as pre-screening tool to increase the prevalence of brain Aβ and tau pathology before confirming the AD diagnostic. These BBM will be also used as main inclusion criterion to select patients for which BBM can achieve sufficiently high diagnostic performance. The BBM will be assessed by using a mass spectrometry-based plasma assay (LC-MS/MS), namely the PrecivityAD™ blood test which is marketed by company C2N (www.PrecivityAD.com). This test simultaneously quantifies plasma amyloid beta (Aβ) 42 and 40 (Aβ42 and Aβ40) concentrations and identifies the presence of plasma Apolipoprotein E (ApoE) isoform-specific peptides isoforms (i.e., ApoE2, ApoE3, ApoE4 isoforms) to determine APOE genotype. The test's statistical algorithm combines the Aβ42/40 ratio, established APOE genotype to determine status of patients' brain amyloidosis. Studies have demonstrated that lower plasma Aβ42/Aβ40 ratio, in combination of the well-established risk factors of APOE4 status and age, correlate with brain amyloidosis as measured using amyloid PET imaging (references 16 and 17). Therefore, the Amyloid Probability Score (APS) based on the plasma Aβ42/40 ratio, APOE genotype (determined by the ApoE peptide isoforms) and patient age will be assessed to select patients having higher probability of brain amyloid burden. Subjects having a Aβ42/40 Ratio ≥0.089, the presence of apoE4 allele, and an APS ranging from 58-100 will be selected (see Table 1 below). The amyloid Probability Score (APS) represents the estimated likelihood from 0 (low likelihood) to 100 (high likelihood) that the patient is currently positive to amyloid PET imaging (presence of amyloid plaques) based on their Aβ42/40 ratio, age, and established APOE genotype.

TABLE 1
Reference intervals

| | |
|---|---|
| Aβ42/40 | <0.089 is consistent with presence of amyloid plaques. |
| ratio | ≥0.089 is consistent with absence of amyloid plaques |
| ApoE | E2/E2; E2/E3; E2/E4; E3/E3; E3/E4; E4/E4 |
| Proteotype | E3 is the most common allele. |
| | E4 allele is associated with increased risk of amyloid |
| | plaques. |
| | E2 allele is associated with lower risk of amyloid plaques |
| Amyloid | 0-100 |
| Probability | Low (0-35): consistent with absence of amyloid plaques |
| | Intermediate (26-57) |
| | High (58-100) consistent with presence of amyloid plaques. |

### Example 5.2: Indications for Use

The non-invasive medical device according to the present invention is in the form of a head-mounted wearable non-invasive device which may be placed on the head, in direct contact with the scalp and/or skull of a subject, thereby allowing homogeneous and reliable exposure of the cortex of the subject to said electromagnetic waves either alone or in combination with red and near-infrared lights. It is self-contained and has been designed for in-home daily treatment, allowing for complete mobility and comfort in performing daily activities during treatment. The device has a custom control panel that is powered by a rechargeable battery. This control panel/battery box may be worn on the upper arm and wired to specialized antennas in the headset worn by the subject. For each day of in-home treatment, the subject wears the headset for two one-hour treatment: 1-hour in the morning and 1-hour in the afternoon or evening with at least a 7-hour rest in between the treatment.

### Example 1.3: Exploratory Clinical Phase and Intended Outcomes and Clinical Benefits for Alzheimer's Disease Patients

It is expected that Alzheimer's patients, most likely at the mild-to-moderate stage may see some improvements in one or more the following outcomes compared to baseline, after at least 2-month treatment with the medical device according to the present invention. This is expected to be reflected in a lesser burden for the caregiver and improved quality of life of the patients and family. In particular, changes from baseline at 2 months into treatment, in one of the following symptoms, mood and behavioral symptoms, depression, anxiety, irritability, inappropriate behavior, sleep disturbance, psychosis, and/or agitation are particularly expected.

### Impact on Mini-Mental State Examination (MMSE)

MMSE is a brief, structured test of mental status that takes about 10 minutes to complete. It involves 11 questions that check for thinking, communication, understanding, and memory impairments. Specifically, the MMSE assesses six areas of mental abilities orientation of time and space, attention and concentration, short-term memory recall, language skills, visuospatial abilities, ability to understand and follow instructions: The person may be given a series of tasks while their ability to follow instructions is evaluated.

Scores on the MMSE range from 0 to 30, with scores of 25 or higher being traditionally considered normal. Scores less than 10 generally indicate severe impairment, while scores between 10 and 20 indicate moderate dementia. People with early-stage Alzheimer's disease tend to score in the 20 to 25 range.

Mild-to-moderate Alzheimer's subjects having above 65 years old and scoring between 16-26 at the MMSE are expected to show some improvements to their cognitive impairment.

### Impact on Alzheimer's Disease Assessment Scale-Cognitive Subscale 14 (ADAS-Cog-Plus or ADAS-Cog14)

Alzheimer's Disease Assessment Scale-Cognitive Subscale 14 (ADAS-Cog14) is one of the most widely used cognitive scales in clinical trials and is the “gold standard” for assessing antidementia treatments. ADAS-Cog14 consists of 14 competencies: word recall, commands, constructional praxis, object and finger naming, ideational praxis, orientation, word recognition, remembering word recognition instructions, comprehension of spoken language, word finding difficulty, spoken language ability, delayed word recall, number cancellation, and maze task. The ADAS-Cog14 scale ranges from 0 to 90. Higher scores indicate greater cognitive impairment.

Alzheimer's disease subjects treated with the medical device according to the present invention are for at least a 2-month are expected to present enhanced cognitive performance. Indeed, compared to baseline, the average performance in the ADAS-cog14 is expected to improve by over 4 points following 2-month treatment.

### Impact on Quality-of-Life Scale in Alzheimer's Disease (QOL-AD)

The QOL-AD is a standard quality of life measure that asks parallel questions of Alzheimer's disease patients and their caregivers. The QOL-AD is a series of questions designed to be administered to individuals with dementia, to obtain a rating of a patient's quality of life from both the patient and the caregiver. It includes assessments of the individual's relationship with friends and family, concerns about finances, physical condition, mood, and an overall assessment of life quality.

### Impact on the Neuropsychiatric Inventory (NPI) Score

NPI is a well-validated, reliable, multi-item instrument to assess psychopathology (e.g., behavioral symptoms) in AD based on a questionnaire completed by the participants' study partners/based on a standardized caregiver interview. NPI assesses the frequency, severity and level of distress caused by 12 common dementia-related behaviors: delusions, hallucinations, agitation/aggression, depression/dysphoria, anxiety, elation/euphoria, apathy/indifference, disinhibition, irritability/lability, aberrant motor behavior, sleep, and appetite/eating.

### Impact on Electroencephalogram's (EEG) and Attention's Span

The EEG recordings will give insight into the effect on the neural activity of the brain. It is recognized that Alzheimer's patients generally experience a slowing of their EEG activity with EEG waves as slow as theta or delta neuronal waves. It is expected that Alzheimer's patients undergoing treatment with the medical device according to the present invention increase their neuronal activity to alpha waves as well as their attention to their direct environment and discussions with their caregivers and family.

### Impact on EQ-5D European Quality of Life Scale

The EQ-5D is a standardized instrument for use as a measure of health outcomes. It includes measures of mobility, self-care, usual activities, pain/discomfort, and anxiety/depression.

Each dimension has 5 levels: no problems, slight problems, moderate problems, severe problems, and extreme problems.

The EQ visual analogue scale (VAS) records the respondent's self-rated health on a 20 cm vertical, visual analogue scale with endpoints labelled ‘the best health you can imagine’ and ‘the worst health you can imagine’. This information can be used as a quantitative measure of health as judged by the individual respondents.

### Impact on ADSL-ADL: Alzheimer's Disease Cooperative Study Activities of Daily Living

ADCS-ADL assesses the competence of patients with AD in basic and instrumental activities of daily living (ADLs). It can be completed by a caregiver in questionnaire format or administered by a clinician/researcher as a structured interview with a caregiver. ADCS-ADL scores range from 0-53, with higher scores indicating greater independence.

### Impact on Sleep Time/Sleep Efficiency

This can be easily assessed by analyzing the sleep data reported by an Actigraph activity monitor. The activity monitor may be attached to a wristband and worn by the subject through the study completion. Treated Alzheimer's disease patients are expected to show less agitation and anxiety in the late afternoon and to experience a better quality of their sleep.

Alzheimer's disease subjects treated with the non-invasive medical device according to the present invention for at least a 2-month are expected to present improvements in one or more of the above tests and scores compared to baseline.

Impact of the treatment on the improvement of the conditions of the Alzheimer's patients may be followed by other clinical endpoints such as change in Caregiver Burden Inventory (CBI), in Positive Aspects of Caregiving Scale, in the clock-drawing test, in the CTT2/CTT1: in the performance on Color Trails Test, in GDS: Geriatric Depression Scale for caregivers, change in Positive Aspects of Caregiving scale, in ADL SAD or ADSC-ADL-sev: Alzheimer's Disease Cooperative Study—Activities of Daily Living for Severe Alzheimer's Disease score, in the Digit Span forward and Digit Span backward score.

### Impact on Digital Biomarkers

Assessment of the impact of the treatment with the medical device according to the present invention may be made by using digital biomarkers. These include real-time, continuous, and non-invasive home-based assessment of health-relevant activity and behavior using the Collaborative Aging Research Using technology (CART) platform developed by the Oregon Center for Aging & Technology (ORCATECH).

The CART platform is a multi-functional digital technology platform allowing to assess in real-time and in a non-obtrusive manner the health and wellness of Alzheimer's patients. The platform comprises ambient technology, wearables, and other sensors installed in the homes of Alzheimer's patients. The sensors include for example passive infrared sensors (motion activity detectors) which monitor the walking speed as well as the amount of time participants spend in each room of their home and how often they move around in their home; actigraphy watches are provided to measure the total activity; electronic pillboxes recording when and which pillbox doors are opened/closed; contact door sensors monitoring the amount of time spent outside of their homes, etc. . . . .

Such variety of home-based sensors are thus used to monitor the impact of the treatment with the medical device according to the present invention inter alia on the walking speed, the sleep activity, activity and social engagement, i.e., time out of the home, mobility patterns of the patients within their homes (e.g., reflecting agitation, apathy, depression), computer use, medication-taking adherence, driving patterns, metadata from online behavior. Alzheimer's patients and their caregivers are expected to notice a decrease in agitation and anxiety, and improvement of sleep, a higher attention span which may improve computer use, social engagement, medication-taking adherence.

### Impact on Fluid Biomarkers Upon Completing at Least 2-Month Treatment

Following plasma levels of a combination of biomarkers allows precise monitoring of the disease progression and treatment response.

Several tests either based on the Elisa technique or mass spectrometry may be used. By way of example, the PrecivityAD2™ blood test from the company C2N (www.PrecivityAD.com) is used to measure plasma Aβ42 and Aβ40, as well as plasma total tau (T-tau), and plasma phosphorylated tau 217 (pTau217) and calculate the plasma Ab42/40, plasma pTau217/non-phospho-tau217 ratio, and Amyloid Probability Score-2. Indeed, the ratio plasma Aβ42/Aβ40 has been shown to reflect amyloid removal. Also, plasma p-tau217 reduction was evidenced to correlate with changes in amyloid and tau load (references 22-23). In addition, plasma biomarkers Aβ42, Aβ40, Glial Fibrillary Acidic Protein (GFAP) and Neurofilament light chain (Nf-L) are measured using the Simoa multiplex Bead-Based Advantage Assays of Quanterix (Neurology 4-Plex E; catalog number 103670) to monitor the effects of the disease modifying treatment at 2 months after treatment.

Decreased levels of Aβ₁₋₄₂ peptide in CSF and increased levels of plasma Aβ₁₋₄₂ peptide occur in conjunction with cognitive decline. The change score may thus be determined by calculating the ratio of plasma Aβ₁₋₄₂ peptide after treatment over baseline. Also, increased levels of Aβ₁₋₄₀ and Aβ₁₋₄₂ soluble monomeric peptides and of oligomeric Aβ aggregates are expected to peak in the blood and cerebrospinal fluid (CSF) of subjects after completion of the 2-month treatment with a medical head device according to the present invention. Therefore, monitoring the change in the ratio of Aβ₁₋₄₂/Aβ₁₋₄₀ may be indicative of the efficiency of the treatment.

Tau protein forms insoluble filaments that accumulate as neurofibrillary tangles (NFT) in AD. At least 2 months of treatment with the non-invasive medical device of the present invention is expected to result in some increase in plasma t-tau (total tau protein) levels, since it induces the dissociation of the tau tangles and thus an increase in monomeric tau within plasma of the treated Alzheimer disease subjects. The change score is determined by calculating the ratio of plasma and CSF tau after treatment over the baseline plasma tau levels.

In the same way following the levels of neurofilament light (NF-L) is indicative of the stabilization and/or reversal of the disease since NF-L is released in significant quantity following axonal damage or neuronal degeneration. The change score is determined by calculating the ratio of plasma or CSF levels of NfL after treatment over the baseline levels of NfL in plasma and CSF, respectively.

### Example 6: Pilot Clinical Phase for Testing Safety and Efficacy of the Non-Invasive Medical Head Device on Mild Cognitive Impairment (MCI) and Amnestic Mild Cognitive Impairment (aMCI)

The objectives are to evaluate cognitive the use of the non-invasive medical device may have neural impacts on MCI and aMCI during a pilot feasibility study. Participants having more than 50 years old and meeting the National Institute on Aging and Alzheimer's Association (NIA-AA) criteria for MCI or aMCI due to Alzheimer's disease may be enrolled. The patients undergo a 2-month trial of the daily treatment at home each for 20 minutes per session. All patients undergo clinical and cognitive assessment, blood sample collection, and structural and resting state functional MRI scans pre and post treatment.

Several outcome measures may be used for monitoring the efficiency of the treatment of patients affected by MCI or aMCI, such as changes from pre- to post-treatment on mental status and cognitive function assessed by Mini-Mental State Examination (MMSE), changes from pre- to post-treatment on verbal learning and memory assessed by California Verbal Learning Test II (CVLTII), changes from pre- to post-treatment on visuospatial memory assessed by Brief Visuospatial Memory Test Revised (BVMT-R), changes from pre- to post-treatment on processing speed assessed by Trail Making Test (TMT)-part A, and/or changes from pre- to post-treatment on Quality of life using QOL-AD.

### Example 7: Pilot Clinical Phase for Testing Safety and Efficacy of the Non-Invasive Medical Head Device on Parkinson's Disease

This is a pilot study of the efficacy of the non-invasive medical head device according to the present invention to slow down the neurodegenerative process, protect dopaminergic neurons, reduce the symptoms patients with Parkinson's Disease (PD) experience, and to enhance cognition and mood in individuals with Parkinson disease. The overall hypothesis is that use of the medical head device has positive effects on brain health, enhancement of neurons, neuroprotection, better cognitive and mood performance.

Patients having more than 18 years old, with Idiopathic Parkinson's disease H & Y 1-3 (Hoehn&Yahr), and MMSE score above 22 may be enrolled.

Parkinson subjects treated with the non-invasive medical device according to the present invention for at least a 2-month are expected to present improvements in one or more of the following outcome measures compared to baseline: Change from baseline in motor clinical signs progression evaluation (Scores on the Movement Disorder Society-Sponsored Revision of the Unified Parkinson's Disease Rating Scale), non-motor behavioral signs progression evaluation (Scores on the non-motor scales Behavioral Evaluation in Parkinson's disease), non-motor clinical signs progression evaluation (Scores on the non-motor scales Lille Apathy Rating Scale), evolution of the quality of life (Parkinson Disease Quotation (PDQ-39) quiz score), assessment of tremor, akinesia and stiffness, speech, walking and balance disorders, walking speed evaluation and of walking parameters (score in the “freezing of gait” questionnaire), as well as ARENA, learning and memory change from baseline to post-testing, learning and memory change from baseline to post-testing.

### Example 8: Pilot Clinical Phase for Testing Safety and Efficacy of the Non-Invasive Medical Head Device on Traumatic Brain Injury

The purpose of this study is to examine effectiveness of an at-home use of the non-invasive medical head device according to the present invention on mild-moderate traumatic brain injury cases. Participants are expected to complete two months of treatment at home 3 times to 6 times a week. Each home treatment is 30 minutes. Participants are randomized. Group 1 receives both a series of sham and a series of real treatments and Group 2 receives two series of real treatments. Sham and Real devices are identical in look and feel, except no or very little electromagnetic signals and photons are emitted from the sham head devices.

Each participant is assigned his/her own medical head device for hygiene reasons. The assigned device will be provided to each participant at a 1-hour in-office training session, after the first Neuropsychological (NP) Testing. Training that includes both verbal and written instructions will be provided, along with demonstration of use of the device. A treatment log, storage box and alcohol wipes for cleaning are provided. The first treatment is completed at the training session.

A staff person will telephone each participant weekly, to fill out a questionnaire about the intervention including inquiring if the treatments are being performed, if the treatment log sheets are being filled out, and note if there are any questions, concerns or problems.

Neuropsychological testing and structural and functional MRI (fMRI) scans are administered to examine behavioral and brain changes before and after the treatment. MRI scans examine some mechanism of treatment including changes in blood flow, functional connectivity and neurochemicals.

Outcome measures include functional MRI: resting-state functional-connectivity Magnetic Resonance Imaging (rs-fc MRI), California Verbal Learning Test (CVLT), Long-Delay Free Recall (LDFR) is the Primary Outcome Measure which examines verbal learning, organization and memory. The subtest LDFR, CVLT-II specifically assesses long-delay (20 min), verbal memory, which can be affected after brain injury.

### Example 9: Pilot Clinical Phase for Testing Safety and Efficacy of the Non-Invasive Medical Head Device on Migraine Headaches

This pilot study aims to validate the efficiency of the non-invasive medical head device for treatment in patients with migraine and tension-type headache symptoms. Various physiological measurements are taken before, during, and after the treatments, including skin type, weight, height, blood pressure, and heart rate. Additionally, data from questionnaires on pain and headache symptoms will be analyzed. This addresses the need for effective pain management strategies in cases where medication-based treatments may have unwanted side effects. Outcome measures include headache pain as assessed using the Visual Analog Scale from zero to 10.

### Example 10: Pilot Clinical Phase for Testing Safety and Efficacy of the Non-Invasive Medical Head Device on Tinnitus

The aim is to determine the effectiveness of the non-invasive medical head device for mitigating and/or alleviating tinnitus. Patients having unilateral tinnitus for at least 3 months may be enrolled. They follow at home treatment with the medical head device for 1 to 2 months, for 20-30 min daily. Efficiency of the treatment is assessed by numerical estimates of tinnitus severity before and after treatment.

### Example 11: Pilot Clinical Phase for Testing Safety and Efficacy of the Non-Invasive Medical Head Device on Depression

The objective is to validate the efficiency of the non-invasive medical head device on a cohort study of patients having depression, refractory to antidepressant drugs and/or with a score on Hamilton Depression Scale (HAM-D17) above 17. Patients may use the medical head device daily for 1 to 2 months, 20-30 min per session. The outcome measures include the change in the Hamilton Depression Scale (HAM-D17), life quality (WHO-5 scale), response and remission.

1. A head device comprising an array of one or more body tissue radiofrequency applicators (50) configured for emitting an electromagnetic signal at a frequency ranging from 30 to 3000 MHz, said array of one or more body tissue radiofrequency applicators (50) being combined with an array of one or more LEDs (200) configured for emitting red and/or near-infrared signals, wherein said one or more body tissue radiofrequency applicators (50) and of one or more LEDs (200) are embedded within said head device, wherein said head device is configured to fit on head of a subject, and wherein said combined arrays of one or more body tissue radiofrequency applicators (50) and of one or more LEDs (200) are positioned such that they are adjacent to said head when the head device is worn by the subject.

2. The head device according to claim 1, wherein the frequency of said electromagnetic signal is in a range of 30-300 MHz, 30-200 MHz, 40-250 MHz, 50-200 MHz, 50-150 MHz, 100-150 MHz, 60-100 MHz, 50-70 MHz, 60-65 MHz, 400-1500 MHz, 500-1300 MHz, 800-1100 MHz, 850-950 MHz, 800-930 MHz, 850-915 MHz, or 910-920 MHz.

3. The head device according to claim 1, wherein said electromagnetic signal is pulsed and wherein the repetition rate of said pulsed electromagnetic signal is between 40-100 Hz, 100-200 Hz, or around 40 Hz, 100 Hz, or 200 Hz.

4. The head device according to claim 1, wherein said red signals have a wavelength in a range of from 620 to 680 nm and said near-infrared signals have a wavelength in a range of from 800 to 1100 nm.

5. The head device according to claim 1, wherein said red signals and near-infrared signals are pulsed with the same or a different repetition rate ranging from 10 Hz to 100 Hz, or from 10-60 Hz, around 20 Hz, or around 40 Hz.

6. The head device according to claim 1, wherein each of said one or more body tissue radiofrequency applicators (50) is divided into at least two sections or into two halves (70), each of these sections or halves (70) comprising a wire or PCB (80) having a first and a second end, and wherein the wire or PCB (80) in each of these at least two sections or halves (70) is connected to a central connector structure (90) having a first conductor structure (100) and a second conductor structure (110), a first end of each of these wires (80) being connected to said first conductor structure (100) and a second end of each of these wires (80) being connected to said second conductor structure (110).

7. The head device according to claim 6, wherein the ends of the wires or PCBs (80) being connected to the inner coaxial or first conductor structure (100) extend unbroken and preferably along a straight line across the central connector structure (90) from one half (70) to the other half (70) and wherein the ends of the wires (80) being connected to the outer coaxial or second conductor structure (110) do not extend unbroken across the central connector structure (90) from one half (70) to the other half (70) but preferably form an interrupted straight line.

8. The head device according to claim 7, wherein the ends of the wires (80) connected to the coaxial connector structure (90), when looking in top view onto said central connector structure (90), are fully crisscrossed forming a X-shaped structure (130).

9. The head device according to claim 6, wherein the ends of the wires (80) being connected to the inner coaxial or first conductor structure (100) extend unbroken along a −Y-shaped line from one half (70) to the another half (70), the ends of the wires or PCBs (80) being connected to the outer coaxial or second conductor structure (110) end at the outer coaxial or second conductor structure (110) thereby forming a Y-shaped structure or line (120) that is interrupted by the central connector structure (90).

10. The head device according to claim 6, wherein said at least two sections or halves (70) of each one or more body tissue radiofrequency applicators (50) are arranged to have a bowtie configuration, a bowtie complex butterfly configuration, or a fully compact meandered configuration.

11. The head device according to claim 1, wherein each of said one or more body tissue radiofrequency applicators (50) are made of wire (80) or PCB, curved flexible PCB, or flexy PCB.

12. The head device according to claim 1, which is a medical or wellness head device.

13. The head device according to claim 1, which is non-invasive brain stimulation head device.

14. The head device according to claim 1, comprising an intelligent wearable cap comprising multiple neurological sensors, processing units, and wireless communication modules.

15. The head device according to claim 14, wherein said neurological sensors comprise electroencephalogram (EEG) electrodes, near-infrared spectroscopy sensors, or magnetometers.

16. The head device according to claim 14, wherein said wireless communication modules comprise Bluetooth, Wi-Fi, or 5G connectivity for real-time data transmission to external devices for real-time analysis.

17. The head device according to claim 14, wherein said processing units comprises means for machine learning algorithm for analyzing brainwave patterns and providing real-time feedback.
